(19) United States
(12) Patent Application Publication
(10) Pub. No.: US 2020/0055908 A1
(43) Pub. Date: Feb. 20, 2020

(54) TRANSGENIC LAND PLANTS COMPRISING ENHANCED LEVELS OF MITOCHONDRIAL TRANSPORTER PROTEIN

(71) Applicant: YIELD10 BIOSCIENCE, INC., Woburn, MA (US)

(72) Inventors: Oliver P. Peoples, Arlington, MA (US); Kristi D. Snell, Belmont, MA (US); Meghna Malik, Saskatoon (CA); Madana M.R. Ambavaram, Norwood, MA (US)

(21) Appl. No.: 16/487,494

(22) PCT Filed: Feb. 22, 2018
(86) PCT No.: PCT/US2018/019105

§ 371 Date: Aug. 21, 2019

Related U.S. Application Data
(60) Provisional application No. 62/462,074, filed on Feb. 22, 2017.

(51) Int. Cl. C07K 14/405; C12N 15/82

(52) U.S. Cl. CPC C07K 14/405; C12N 15/8261; C12N 15/8247

(57) ABSTRACT

A transgenic land plant is provided. The transgenic land plant comprises a mitochondrial transporter protein of a eukaryotic algae. The mitochondrial transporter protein of the eukaryotic algae is heterologous with respect to the transgenic land plant. The mitochondrial transporter protein is a sequence or ortholog of CCP1 of Chlamydomonasreinhardtii, a mitochondrial transporter protein of Chlorella sorokiniana, a mitochondrial transporter protein of Chlorella variabilis, a mitochondrial transporter protein of Chondrus crispus, a mitochondrial transporter protein of Gonium pectorale, or a mitochondrial transporter protein of Volvox carteri. The mitochondrial transporter protein is localized to mitochondria of the transgenic land plant based on a mitochondrial targeting signal intrinsic to the mitochondrial transporter protein. The mitochondrial transporter protein is localized to mitochondria of the transgenic land plant based on a mitochondrial targeting signal intrinsic to the mitochondrial transporter protein and is expressed predominantly in seeds of the transgenic land plant.

### FIELD OF THE INVENTION

The present invention relates generally to transgenic land plants, and more particularly, to transgenic land plants comprising a mitochondrial transporter protein of a eukaryotic algae that is expressed predominantly in seeds of the transgenic land plant.

### BACKGROUND OF THE INVENTION

The world faces a major challenge in the next 35 years to meet the increased demands for food production to feed a growing global population, which is expected to reach 9 billion by the year 2050. Food output will need to be increased by up to 70% in view of the growing population. Increased demand for improved diet, concomitant land use changes for new living space and infrastructure, alternative uses for crops and changing weather patterns will add to the challenge.

Major agricultural crops include food crops, such as maize, wheat, oats, barley, soybean, millet, sorghum, pulses, bean, tomato, corn, rice, cassava, sugar beets, and potatoes, forage crop plants, such as hay, alfalfa, and silage corn, and oilseed crops, such as camelina, Brassica species (e.g. B. napus (canola), B. rapa, B. juncea, and B. carinata), crambe, soybean, sunflower, safflower, oil palm, flax, and cotton, among others. Productivity of these crops, and others, is limited by numerous factors, including for example relative inefficiency of photochemical conversion of light energy to fixed carbon during photosynthesis, as well as loss of fixed carbon by photorespiration and/or other essential metabolic pathways having enzymes catalyzing decarboxylation reactions. Crop productivity is also limited by the availability of water. Current crop production relies primarily on crop species that were bred by conventional means for improved yield which was improved by continuous incremental changes over many years. Over this period any step changes in yield were typically enabled by new technologies such as the advent of nitrogen fertilizers, improving the harvest index (the ratio of harvestable seed to biomass) as for example dwarf wheat and rice varieties, hybrids such as corn, canola and rice with “hybrid vigor,” and more recently, improved agronomic practices such as increased density of seed planting enabled in part by transgenic input traits including herbicide resistance and pesticide resistance. Unfortunately, given the inherent complexity of plant metabolism and the fact that plants have evolved to balance inputs with growth and reproduction, it is likely that achieving further step changes in crop yield will require new approaches.

It has recently been shown Schnell et al., WO 2015/103074 that Camelina plants transformed to express CCP1 of the algal species Chlamydomonas reinhardtii have reduced transpiration rates, increased CO₂ assimilation rates and higher yield than control plants which do not express the CCP1 gene. CCP1 was originally identified as a bicarbonate transporter (Ci), and was presumed to locate to the chloroplast membrane where it would function to transport bicarbonate from the cytosol into the chloroplast, thereby increasing the CO₂ concentration for RUBISCO. More recently, Atkinson et al., (2015) Plant Biotechnol. J., doi: 10.1111/pbi.12497, discloses that CCP1 and its homolog CCP2, which were characterized as Ci transporters, previously reported to be in the chloroplast envelope, localized to mitochondria in both Chlamydomonas reinhardtii, as expressed naturally, and tobacco, when expressed heterologously, suggesting that the model for the carbon-concentrating mechanism of eukaryotic algae needs to be expanded to include a role for mitochondria. Atkinson et al. (2015) disclosed that expression of individual Ci transporters did not enhance growth of the plant Arabidopsis, and suggests that stacking of further components of carbon-concentrating mechanisms will probably be required to achieve a significant increase in photosynthetic efficiency.

In co-pending Patent Application PCT/US2017/016421 to Yield10 Bioscience a number of orthologs of CCP1 from algal species that share common protein sequence domains including mitochondrial membrane domains and transporter protein domains were shown to increase seed yield in Camelina plants. Schnell et al., WO 2015/103074, also reported a decrease in seed size in higher yielding Camelina lines expressing CCP1 constitutively. Both groups expressed the CCP1 and/or algal ortholog genes under the control of constitutive plant promoters where they are expressed throughout the life cycle of the plant and in most plant tissues including seed. Also in co-pending Patent Application PCT/US2017/016421 to Yield10 Bioscience, CCP1 and its orthologs from algae were described as putative bicarbonate transporter genes to reflect the reality that the function of these proteins has not previously been determined and their initial designation as Ci proteins was assumed based on the increased expression of CCP1 in Chlamydomonas under CO₂ limiting conditions. Herein we refer to CCP1 and its orthologs from other eukaryotic algae as mitochondrial transporters. It would have been reasonable to assume that the expression of CCP1 in seed would be detrimental to seed metabolism and development, limiting the potential increase in seed yield that may be achievable from the increased carbon assimilation rate demonstrated in the transgenic CCP1 plants. In addition smaller seed size may negatively impact the adoption of these plants for large scale agriculture due to impacts on planting, harvesting and processing equipment

Thus, there is a need for improvements to transgenic plants having enhanced carbon capture systems based on increased expression of mitochondrial transporters such as CCP1 or its orthologs to reduce negative impacts such as smaller seed size and/or to further improve seed yield. In order to develop methods to overcome this limitation the inventors sought to gain a better scientific understanding of the observed negative effect from constitutive expression of CCP1 on seed size. The inventors therefore tested the impact of expressing CCP1 or any of its orthologs using seed-specific promoters with the unexpected outcome that both seed yield and seed size increased.

Provided herein are eukaryotic algal mitochondrial transporter genes, and proteins. Also provided herein are genetic constructs for expressing the eukaryotic algal mitochondrial transporter genes in a seed-specific manner in plants wherein the plants have increased seed yield with no reduction in seed size as compared to plants not expressing the eukaryotic algal mitochondrial transporter genes or expressing the eukaryotic algal mitochondrial transporter genes in a constitutive manner. Also provided herein are plants expressing eukaryotic algal mitochondrial transporter genes in both a seed-specific and a constitutive manner wherein the eukaryotic algal mitochondrial transporter genes may be the same or different genes, from the same algal species or from different algal species.

### BRIEF SUMMARY OF THE INVENTION

In accordance with one aspect of the present invention, a transgenic land plant is disclosed. The transgenic land plant comprises a mitochondrial transporter protein of a eukaryotic algae. The mitochondrial transporter protein of the eukaryotic algae is heterologous with respect to the transgenic land plant. The mitochondrial transporter protein is a sequence or ortholog of (a) CCP1 of Chlamydomonas reinhardtii of SEQ ID NO: 1, (b) a mitochondrial transporter protein of Chlorella sorokiniana of SEQ ID NO: 2, (c) a mitochondrial transporter protein of Chlorella variabilis of SEQ ID NO: 3, SEQ ID NO: 4, SEQ ID NO: 5, or SEQ ID NO: 6, (d) a mitochondrial transporter protein of Chondrus crispus of SEQ ID NO: 7, SEQ ID NO: 8, or SEQ ID NO: 9, (e) a mitochondrial transporter protein of Gonium pectorale of SEQ ID NO: 19, or SEQ ID NO: 20, or (f) a mitochondrial transporter protein of Volvox carteri of SEQ ID NO: 21. The mitochondrial transporter protein is localized to mitochondria of the transgenic land plant based on a mitochondrial targeting signal intrinsic to the mitochondrial transporter protein. The mitochondrial transporter protein is expressed predominantly in seeds of the transgenic land plant.

### BRIEF DESCRIPTION OF THE DRAWINGS

FIG. 1 shows predicted transmembrane regions (grey shading) of CCP1 protein of Chlamydomonas reinhardtii of SEQ ID NO: 1, based on Phobius prediction. Data correspond to plots of posterior label probability (y-axis) versus amino acid number of the protein (x-axis), including predicted transmembrane regions (grey shading), cytoplasmic regions (Xs on grey line), non-cytoplasmic regions (filled circles on black line), and signal peptides (open triangles on grey line).

FIG. 2 shows predicted transmembrane regions (grey shading) of a protein of Chlorella sorokiniana (GAPD01006726.1) of SEQ ID NO: 2 that is an ortholog of CCP1, based on Phobius prediction. Data correspond to plots of posterior label probability (y-axis) versus amino acid number of the protein (x-axis), including predicted transmembrane regions (grey shading), cytoplasmic regions (Xs on grey line), non-cytoplasmic regions (filled circles on black line), and signal peptides (open triangles on grey line).

FIG. 3 shows predicted transmembrane regions (grey shading) of a protein of Chlorella variabilis (XM_005846489.1) of SEQ ID NO: 6 that is an ortholog of CCP1, based on Phobius prediction. Data correspond to plots of posterior label probability (y-axis) versus amino acid number of the protein (x-axis), including predicted transmembrane regions (grey shading), cytoplasmic regions (Xs on grey line), non-cytoplasmic regions (filled circles on black line), and signal peptides (open triangles on grey line).

FIG. 4 shows predicted transmembrane regions (grey shading) of a protein of Chlorella variabilis (XM_005852157.1) of SEQ ID NO: 4 that is an ortholog of CCP1, based on Phobius prediction. Data correspond to plots of posterior label probability (y-axis) versus amino acid number of the protein (x-axis), including predicted transmembrane regions (grey shading), cytoplasmic regions (Xs on grey line), non-cytoplasmic regions (filled circles on black line), and signal peptides (open triangles on grey line).

FIG. 5 shows predicted transmembrane regions (grey shading) of a protein of Chlorella variabilis XM_005843001.1 of SEQ ID NO: 5 that is an ortholog of CCP1, based on Phobius prediction. Data correspond to plots of posterior label probability (y-axis) versus amino acid number of the protein (x-axis), including predicted transmembrane regions (grey shading), cytoplasmic regions (Xs on grey line), non-cytoplasmic regions (filled circles on black line), and signal peptides (open triangles on grey line).

FIG. 6 shows predicted transmembrane regions (grey shading) of CCP1 protein of Gonium pectorals of SEQ ID NO: 19, based on Phobius prediction. Data correspond to plots of posterior label probability (y-axis) versus amino acid number of the protein (x-axis), including predicted transmembrane regions (grey shading), cytoplasmic regions (Xs on grey line), non-cytoplasmic regions (filled circles on black line), and signal peptides (open triangles on grey line).

FIG. 7 shows predicted transmembrane regions (grey shading) of CCP1 protein of Gonium pectorale of SEQ ID NO: 20, based on Phobius prediction. Data correspond to plots of posterior label probability (y-axis) versus amino acid number of the protein (x-axis), including predicted transmembrane regions (grey shading), cytoplasmic regions (Xs on grey line), non-cytoplasmic regions (filled circles on black line), and signal peptides (open triangles on grey line).

FIG. 8 shows predicted transmembrane regions (grey shading) of CCP1 protein of Volvox carteri f. nagariensis of SEQ ID NO: 21, based on Phobius prediction. Data correspond to plots of posterior label probability (y-axis) versus amino acid number of the protein (x-axis), including predicted transmembrane regions (grey shading), cytoplasmic regions (Xs on grey line), non-cytoplasmic regions (filled circles on black line), and signal peptides (open triangles on grey line).

FIG. 9A-C shows a multiple sequence alignment of CCP1 of Chlamydomonas reinhardtii and eleven orthologs of CCP1 of algae, according to CLUSTAL O(1.2.4).

FIG. 10A-B shows plasmid maps of transformation vectors pMBXO85 (SEQ ID NO: 10) and pMBXO86 (SEQ ID NO: 11). Plasmid pMBXO85 contains a constitutive expression cassette, driven by the CaMV35S promoter, for expression of an ortholog of CCP1 gene from an algae Chlorella sorokiniana. An expression cassette for the bar gene, driven by the CaMV35S promoter, imparts transgenic plants resistance to the herbicide bialophos. Plasmid pMBXO86 contains a constitutive expression cassette, driven by the CaMV35S promoter, for expression of an ortholog of CCP1 gene from an algae Chlorella variabilis. An expression cassette for the bar gene, driven by the CaMV35S promoter, imparts transgenic plants resistance to the herbicide bialophos.

FIG. 11A-C shows plasmid maps of transformation vectors pMBXO84 (SEQ ID NO: 12), pMBXO71 (SEQ ID NO: 13), and pMBXO107 (SEQ ID NO: 14). Plasmid pMBXO84 contains a seed-specific expression cassette, driven by the promoter from the soya bean oleosin isoform A gene, for expression of CCP1 from Chlamydomonas reinhardtii. An expression cassette for the bar gene, driven by the CaMV35S promoter, imparts transgenic plants resistance to the herbicide bialophos. Plasmid pMBXO71 contains a seed-specific expression cassette, driven by the promoter from the Arabidopsis thaliana sucrose synthase gene, for expression of CCP1 from Chlamydomonas reinhardtii. An expression cassette for the bar gene, driven by the CaMV35S promoter, imparts transgenic plants resistance to the herbicide bialophos. Plasmid pMBXO107 contains a seed-specific expression cassette, driven by the promoter from the conlinin gene of flax (US 20070192902 A1), for expression of CCP1 from Chlamydomonas reinhardtii. An expression cassette for the bar gene, driven by the CaMV35S promoter, imparts transgenic plants resistance to the herbicide bialophos.

FIG. 12 shows a plasmid map for pMBXO75 (SEQ ID NO: 15). Linear plasmid pMBXO75 contains a seed-specific expression cassette, driven by the promoter from the soya bean oleosin isoform A gene, for expression of CCP1 from Chlamydomonas reinhardtii. The CCP1 gene is codon optimized for soybean. The 2.2 kb, Smal Oleosin-CCP1-oleosin terminator fragment was co-bombarded with a hygromycin cassette in soybean embryogenic cultures.

FIG. 13 shows relative expression levels of the CCP1 transgene in embryos of soybean transformed with pMBXO75. Expression levels were normalized with an internal control gene. The event name and the embryo stage are indicated on the x-axis. The term “pro” indicates proembryos from liquid culture. The term “x-wk gelrite”, where x is a number between 5 and 16, indicates the amount of time that the embryo was incubated on gelrite medium before analysis. Stars indicate lines from which seeds have been harvested. Expression of CCP1 was detected in transgenic embryos from transformants of pMBXO75 but not from wild-type soybean embryos (data not shown).

FIG. 14A-C shows plasmid maps of rice transformation vectors pMBXS1089 (SEQ ID NO: 16), pMBXS1090 (SEQ ID NO: 17), and pMBXS1091 (SEQ ID NO: 18). Plasmid pMBXS1089 contains an expression cassette for the CCP1 gene from Chlamydomonas reinhardtii fused to a C-terminal myc tag (ccpl-myc) possessing the amino acid sequence EQKLISEEDL. The expression of the ccpl-myc gene is controlled by the promoter from the rice ADP-glucose pyrophosphorylase (AGPase) gene (GenBank: AY427566.1, LOC_Os01g44220). An expression cassette for the hptII gene, driven by the CaMV35S promoter and including the hsp70 intron as well as an intron from the bean catalase -1 gene (CAT-1) imparts transgenic plants resistance to the herbicide hygromycin. Plasmid pMBXS1090 contains an expression cassette for CCP1 from Chlamydomonas reinhardtii fused to a C-terminal myc tag. The expression of the ccpl-myc gene is controlled by the promoter from the rice glutelin C (GluC) gene (GenBank: EU264107.1, LOC_Os02g25640). Plasmid pMBXS1091 contains an expression cassette for CCP1 from Chlamydomonas reinhardtii fused to a C-terminal myc tag. The expression of the ccpl-myc gene is controlled by the promoter from the rice beta-fructofuranosidase insoluble isoenzyme 1 (CIN1) gene (LOC_Os02g33110).

FIG. 15 shows a model for further enhanced yield based on inhibiting expression of cell wall invertase inhibitor that would otherwise be upregulated in CCP1 lines.

### DETAILED DESCRIPTION OF THE INVENTION

A transgenic land plant is disclosed. The transgenic land plant comprises a mitochondrial transporter protein of a eukaryotic algae. The mitochondrial transporter protein of the eukaryotic algae is heterologous with respect to the transgenic land plant. The mitochondrial transporter protein is a sequence or ortholog of (a) CCP1 of Chlamydomonas reinhardtii of SEQ ID NO: 1, (b) a mitochondrial transporter protein of Chlorella sorokiniana of SEQ ID NO: 2, (c) a mitochondrial transporter protein of Chlorella variabilis of SEQ ID NO: 3, SEQ ID NO: 4, SEQ ID NO: 5, or SEQ ID NO: 6, (d) a mitochondrial transporter protein of Chondrus crispus of SEQ ID NO: 7, SEQ ID NO: 8, or SEQ ID NO: 9, (e) a mitochondrial transporter protein of Gonium pectorale of SEQ ID NO: 19, or SEQ ID NO: 20, or (f) a mitochondrial transporter protein of Volvox carteri of SEQ ID NO: 21. The mitochondrial transporter protein is localized to mitochondria of the transgenic land plant based on a mitochondrial targeting signal intrinsic to the mitochondrial transporter protein. The transgenic land plant is expressed predominantly in seeds of the transgenic land plant.

Without wishing to be bound by theory, it is believed that modifying a land plant to express a mitochondrial transporter protein of a eukaryotic algae to obtain a transgenic land plant, wherein the mitochondrial transporter protein of the eukaryotic algae is heterologous with respect to the transgenic land plant, is a sequence or ortholog of (a) CCP1 of Chlamydomonas reinhardtii of SEQ ID NO: 1, (b) a mitochondrial transporter protein of Chlorella sorokiniana of SEQ ID NO: 2, (c) a mitochondrial transporter protein of Chlorella variabilis of SEQ ID NO: 3, SEQ ID NO: 4, SEQ ID NO: 5, or SEQ ID NO: 6, (d) a mitochondrial transporter protein of Chondrus crispus of SEQ ID NO: 7, SEQ ID NO: 8, or SEQ ID NO: 9, (e) a mitochondrial transporter protein of Gonium pectorale of SEQ ID NO: 19, or SEQ ID NO: 20, or (f) a mitochondrial transporter protein of Volvox carteri of SEQ ID NO: 21, is localized to mitochondria of the transgenic land plant based on a mitochondrial targeting signal intrinsic to the mitochondrial transporter protein, and is expressed predominantly in seeds of the transgenic land plant, i.e. in a seed-specific manner, will result in enhanced yield without a reduction in seed size, based for example on an increased CO₂ assimilation rate and/or a decreased transpiration rate of the transgenic land plant, in comparison to a reference land plant not expressing the mitochondrial transporter protein, or expressing the mitochondrial transporter protein constitutively. It is believed that the mitochondrial transporter protein will enhance transport of bicarbonate or other metabolites from or into the mitochondria, thereby enabling enhanced rates of carbon fixation by increasing CO₂ recovery from photorespiration and respiration. Moreover, it is believed that by modifying the land plant to express a mitochondrial transporter protein that is localized to mitochondria in particular, it will be possible to stack expression of the mitochondrial transporter protein with expression of other proteins in deliberate and complementary approaches to further enhance yield. In addition, it is believed that by modifying the land plant to express a mitochondrial transporter protein in a seed-specific manner in particular, it will be possible to generate transgenic crops with enhanced yield without a reduction in seed size.

As noted, a transgenic land plant is disclosed. A land plant is a plant belonging to the plant subkingdom Embryophyta.

The term “land plant” includes mature plants, seeds, shoots and seedlings, and parts, propagation material, plant organ tissue, protoplasts, callus and other cultures, for example cell cultures, derived from plants belonging to the plant subkingdom Embryophyta, and all other species of groups of plant cells giving functional or structural units, also belonging to the plant subkingdom Embryophyta. The term “mature plants” refers to plants at any developmental stage beyond the seedling. The term “seedlings” refers to young, immature plants at an early developmental stage.

Land plants encompass all annual and perennial monocotyldedonous or dicotyledonous plants. Preferred dicotyledonous plants are selected in particular from the dicotyledonous crop plants such as, for example, Asteraceae such as sunflower, tagetes or calendula and others; Compositae, especially the genus Lactuca, very particularly the species sativa (lettuce) and others; Cruciferae, particularly the genus Brassica, very particularly the species napus (oilseed rape), campestris (beet), oleracea cv Tastie (cabbage), oleracea cv Snowball Y (cauliflower) and oleracea cv Emperor (broccoli) and other cabbages; cress or canola and others; Cucurbitaceae such as melon, pumpkin/squash or zucchini and others; Leguminosae, particularly the genus Glycine, very particularly the species max (soybean), soya, and alfalfa, pea, beans or peanut and others; Rubiaceae, preferably the subclass Lamiidae such as, for example Coffea arabica or Coffea liberica (coffee bush) and others; Solanaceae, particularly the genus Lycopersicon, very particularly the species esculentum (tomato), the genus Solanum, very particularly the species tuberosum (potato) and melongena (aubergine) and the genus Capsicum, very particularly the genus annuum (pepper) and tobacco or paprika and others; Sterculiaceae, preferably the subclass Dilleniidae such as, for example, Theobroma cacao (cacao bush) and others; Theaceae, preferably the subclass Dilleniidae such as, for example, Camellia sinensis or Thea sinensis (tea shrub) and others; Umbelliferae, particularly the genus Daucus (very particularly the species carota (carrot)) and Apium (very particularly the species graveolens dulce (celery)) and others; and linseed, cotton, hemp, flax, cucumber, spinach, carrot, sugar beet and the various tree, nut and grapevine species, in particular banana and kiwi fruit. Preferred moncotyledonous plants include maize, rice, wheat, sugarcane, sorghum, oats and barley.

In oilseed plants of interest the oil is accumulated in the seed and can account for greater than 10%, greater than 15%, greater than 18%, greater than 25%, greater than 35%, greater than 50% by weight of the weight of dry seed. Oil crops encompass by way of example: Borago officinalis (borage); Camelina (false flax); Brassica species such as B. campestris, B. napus, B. rapa, B. carinata (mustard, oilseed rape or turnip rape); Cannabis sativa (hemp); Carthamus tinctorius (safflower); Cocos nucifera (coconut); Crambe abyssinica (crambe); Cuphea species; Elaeis guinensis (African oil palm); Elaeis oleifera (American oil palm); Glycine max (soybean); Gossypium hirsutum (American cotton); Gossypium barbadense (Egyptian cotton); Gossypium herbaceum (Asian cotton); Helianthus annuus (sunflower); Jatropha curcas (jatropha); Linum usitatissimum (linseed or flax); Oenothera biennis (evening primrose); Olea europaea (olive); Oryza sativa (rice); Ricinus communis (castor); Sesamum indicum (sesame); Thlaspi caerulescens (pennycress); Triticum species (wheat); Zea mays (maize), and various nut species such as, for example, walnut or almond.

Camelina is a very useful system for developing new tools and transgenic approaches to enhancing the yield of crops in general and for enhancing the yield of seed and seed oil in particular. Demonstrated transgene improvements in Camelina can then be deployed in other major crops including canola, soybean, corn, rice, wheat, oats, barley, rye, potato, sweet potato, cassava, cotton, sunflower, safflower, sorghum, millet, lentils, pulses and beans.

As will be apparent, the land plant can be a C3 plant, i.e. a plant in which RubisCO catalyzes carboxylation of ribulose-1,5-bisphosphate by use of CO₂ drawn directly from the atmosphere, such as for example, wheat, oat, and barley, among others. The land plant also can be a C4 plant, i.e. a plant in which RubisCO catalyzes carboxylation of ribulose-1,5-bisphosphate by use of CO₂ shuttled via malate or aspartate from mesophyll cells to bundle sheath cells, such as for example maize, millet, and sorghum, among others.

Accordingly, in some examples the transgenic land plant is a C3 plant. Also, in some examples the transgenic land plant is a C4 plant. Also, in some examples the transgenic land plant is a food crop plant selected from the group consisting of maize, rice, wheat, oat, barley, soybean, millet, sorghum, potato, pulse, bean, and tomato. Also, in some examples the transgenic land plant is a forage crop plant selected from the group consisting of hay, alfalfa, and silage corn. Also, in some examples the transgenic land plant is an oilseed crop plant selected from the group consisting of camelina, Brassica species (e.g. B. napus (canola), B. rapa, B. juncea, and B. carinata), crambe, soybean, sunflower, safflower, oil palm, flax, and cotton.

The transgenic land plant comprises a mitochondrial transporter protein of a eukaryotic algae. A mitochondrial transporter protein is a protein that transports bicarbonate or other metabolites by any transport mechanism into or out of the mitochondria. Mitochondrial transporter proteins include bicarbonate transporters. Classes of bicarbonate transport proteins include anion exchangers and Na⁺/HCO₃⁻¹ symporters.

As noted, the transgenic land plant comprises a mitochondrial transporter protein of a eukaryotic algae. A eukaryotic algae is an aquatic plant, ranging from a microscopic unicellular form, e.g. a single-cell algae, to a macroscopic multicellular form, e.g. a seaweed, that includes chlorophyll a and, if multicellular, a thallus not differentiated into roots, stem, and leaves, and that is classified as chlorophyta (also termed green algae), rhodophyta (also termed red algae), or phaeophyta (also termed brown algae). Some also are generally recognized as a typical and suitable component of a human diet. Eukaryotic algae include, for example, single-cell algae, including the chlorophyta Chlorella sorokiniana and Chlorella variabilis. Eukaryotic algae also include, for example, seaweed, including the chlorophyta Ulva lactuca (also termed sea lettuce) and Enteromorpha (Ulva) intenstinalis (also termed sea grass), the rhodophyta Chondrus crispus (also termed Irish moss or carrigeen), Porphyra umbilicalis (also termed nori), and Palmaria palmata (also termed dulse or dillisk), and the phaeophyta Ascophyllum nodosum (also termed egg wrack), Laminaria digitata (also termed kombu/konbu), Laminaria saccharina (also termed royal or sweet kombu), Himanthalia elongata (also termed sea spaghetti), and Undaria pinnatifida (also termed wakame).

The mitochondrial transporter protein of the eukaryotic algae is heterologous with respect to the transgenic land plant. By this it is meant that the mitochondrial transporter protein of the eukaryotic algae is not normally expressed or otherwise present in land plants of the type from which the transgenic land plant is derived, i.e. land plants of the type from which the transgenic land plant is derived do not express any protein having an amino acid sequence identical to that of the mitochondrial transporter protein of the eukaryotic algae. Rather, the transgenic land plant comprises the mitochondrial transporter protein of the eukaryotic algae based on genetic modification of a land plant to express the mitochondrial transporter protein of the eukaryotic algae, thus resulting in the transgenic land plant.

The mitochondrial transporter protein is a sequence or ortholog of (a) CCP1 of Chlamydomonas reinhardtii of SEQ ID NO: 1, (b) a mitochondrial transporter protein of Chlorella sorokiniana of SEQ ID NO: 2, (c) a mitochondrial transporter protein of Chlorella variabilis of SEQ ID NO: 3, SEQ ID NO: 4, SEQ ID NO: 5, or SEQ ID NO: 6, (d) a mitochondrial transporter protein of Chondrus crispus of SEQ ID NO: 7, SEQ ID NO: 8, or SEQ ID NO: 9, (e) a mitochondrial transporter protein of Gonium pectorale of SEQ ID NO: 19, or SEQ ID NO: 20, or (f) a mitochondrial transporter protein of Volvox carteri of SEQ ID NO: 21. The term “sequence” means a full-length sequence or a partial sequence of a polynucleotide sequence or polypeptide sequence as specified, that has a function associated with the full-length sequence as specified. The term “ortholog” means a polynucleotide sequence or polypeptide sequence possessing a high degree of homology, i.e. sequence relatedness, to a subject sequence and being a functional equivalent of the subject sequence, wherein the sequence that is orthologous is from a species that is different than that of the subject sequence. Homology may be quantified by determining the degree of identity and/or similarity between the sequences being compared.

As used herein, “percent homology” of two polynucleotide sequences or of two polypeptide sequences is determined using the algorithm of Karlin and Altschul (1990), Proc. Natl. Acad. Sci., U.S.A. 87: 2264-2268. Such an algorithm is incorporated into the NBLAST and XBLAST programs of Altschul et al. (1990), J. Mol. Biol. 215: 403-410. BLAST nucleotide searches are performed with the NBLAST program, score=100, word length 12, to obtain nucleotide sequences homologous to a reference polynucleotide sequence. BLAST protein searches are performed with the XBLAST program, score=50, word length=3, to obtain amino acid sequences homologous to a reference polypeptide sequence. To obtain gapped alignments for comparison purposes, Gapped BLAST is utilized as described in Altschul et al. (1997), Nucleic Acids Res. 25: 3389-3402. When utilizing BLAST and Gapped BLAST programs, the default parameters are typically used.

In the case of polypeptide sequences that are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine.

Where a particular polypeptide is said to have a specific percent identity to a reference polypeptide of a defined length, the percent identity is relative to the reference peptide. Thus, a peptide that is 50% identical to a reference polypeptide that is 100 amino acids long can be a 50 amino acid polypeptide that is completely identical to a 50 amino acid long portion of the reference polypeptide. It might also be a 100 amino acid long polypeptide that is 50% identical to the reference polypeptide over its entire length. Many other polypeptides will meet the same criteria.

For reference, as discussed above CCP1 is a mitochondrial transporter of Chlamydomonas reinhardtii. In addition, CCP1 has an amino acid sequence in accordance with SEQ ID NO: 1. Accordingly, in some embodiments, the mitochondrial transporter protein is a full-length sequence of CCP1 of Chlamydomonas reinhardtii of SEQ ID NO: 1, having the function of full-length CCP1. Also in some embodiments, the mitochondrial transporter protein is a partial sequence of CCP1 of Chlamydomonas reinhardtii of SEQ ID NO: 1, also having the function of full-length CCP1. Also in some embodiments, the mitochondrial transporter protein is a polypeptide sequence possessing a high degree of sequence relatedness to CCP1 of Chlamydomonas reinhardtii of SEQ ID NO: 1 and being a functional equivalent thereof, wherein the mitochondrial transporter protein is from a species that is different than Chlamydomonas reinhardtii.

Also for reference, as discussed in detail below, a mitochondrial transporter protein of Chlorella sorokiniana of SEQ ID NO: 2, a mitochondrial transporter protein of Chlorella variabilis of SEQ ID NO: 3, SEQ ID NO: 4, SEQ ID NO: 5, or SEQ ID NO: 6, a mitochondrial transporter protein of Chondrus crispus of SEQ ID NO: 7, SEQ ID NO: 8, or SEQ ID NO: 9, a mitochondrial transporter protein of Gonium pectorals of SEQ ID NO: 19, or SEQ ID NO: 20, and a mitochondrial transporter protein of Volvox carteri of SEQ ID NO: 21 are orthologs of CCP1 of Chlamydomonas reinhardtii. Accordingly, in some embodiments, the mitochondrial transporter protein is a full-length sequence of the mitochondrial transporter protein of Chlorella sorokiniana of SEQ ID NO: 2, the mitochondrial transporter protein of Chlorella variabilis of SEQ ID NO: 3, SEQ ID NO: 4, SEQ ID NO: 5, or SEQ ID NO: 6, the mitochondrial transporter protein of Chondrus crispus of SEQ ID NO: 7, SEQ ID NO: 8, or SEQ ID NO: 9, the mitochondrial transporter protein of Gonium pectorale of SEQ ID NO: 19, or SEQ ID NO: 20, or the mitochondrial transporter protein of Volvox carteri of SEQ ID NO: 21, having the function of the respective full-length mitochondrial transporter protein. Also in some embodiments, the mitochondrial transporter protein is a partial sequence of the mitochondrial transporter protein of Chlorella sorokiniana of SEQ ID NO: 2, the mitochondrial transporter protein of Chlorella variabilis of SEQ ID NO: 3, SEQ ID NO: 4, SEQ ID NO: 5, or SEQ ID NO: 6, the mitochondrial transporter protein of Chondrus crispus of SEQ ID NO: 7, SEQ ID NO: 8, or SEQ ID NO: 9, the mitochondrial transporter protein of Gonium pectorale of SEQ ID NO: 19, or SEQ ID NO: 20, or the mitochondrial transporter protein of Volvox carteri of SEQ ID NO: 21, also having the function of the respective full-length mitochondrial transporter protein. Also in some embodiments, the mitochondrial transporter protein is a polypeptide sequence possessing a high degree of sequence relatedness to one or more of the mitochondrial transporter protein of Chlorella sorokiniana of SEQ ID NO: 2, the mitochondrial transporter protein of Chlorella variabilis of SEQ ID NO: 3, SEQ ID NO: 4, SEQ ID NO: 5, or SEQ ID NO: 6, the mitochondrial transporter protein of Chondrus crispus of SEQ ID NO: 7, SEQ ID NO: 8, or SEQ ID NO: 9, the mitochondrial transporter protein of Gonium pectorale of SEQ ID NO: 19, or SEQ ID NO: 20, or the mitochondrial transporter protein of Volvox carteri of SEQ ID NO: 21, and being a functional equivalent thereof, wherein the mitochondrial transporter protein is from a species that is different than Chlorella sorokiniana, Chlorella variabilis, and/or Chondrus crispus.

The mitochondrial transporter protein is localized to mitochondria of the transgenic land plant based on a mitochondrial targeting signal intrinsic to the mitochondrial transporter protein. The mitochondrial transporter protein can be localized to mitochondria for example based on being encoded by DNA present in the nucleus of a plant cell, synthesized in the cytosol of the plant cell, targeted to the mitochondria of the plant cell, and inserted into outer membranes and/or inner membranes of the mitochondria. A mitochondrial targeting signal is a portion of a polypeptide sequence that targets the polypeptide sequence to mitochondria. A mitochondrial targeting signal intrinsic to the mitochondrial transporter protein is a mitochondrial targeting signal that is integral to the mitochondrial transporter protein, e.g. based on occurring naturally at the N-terminal end of the mitochondrial transporter protein or in discrete segments along the mitochondrial transporter protein. This is in contrast, for example, to fusion of a heterologous mitochondrial targeting signal to a mitochondrial transporter protein that would not otherwise be targeted to mitochondria. For reference, also as discussed above CCP1 is localized to mitochondria in both Chlamydomonas reinhardtii, as expressed naturally, and tobacco, when expressed heterologously. Accordingly, the mitochondrial transporter protein can be a mitochondrial transporter protein that is encoded by nuclear DNA, synthesized cytosolically, targeted to the mitochondria, and inserted into outer membranes and/or inner membranes thereof, based on targeting by a portion of the polypeptide sequence integral to the mitochondrial transporter protein.

Suitable mitochondrial transporter proteins can be identified, for example, based on searching databases of polynucleotide sequences or polypeptide sequences for orthologs of CCP1 of Chlamydomonas reinhardtii of SEQ ID NO: 1, wherein the polynucleotide sequences or polypeptide sequences being derived from eukaryotic algae. Such searches can be carried out, for example, by use of BLAST, e.g. tblastn, and databases including translated polynucleotides, whole genome shotgun sequences, and/or transcriptome assembly sequences, among other sequences and databases, as discussed above. Potential orthologs of CCP1 may be identified, for example, based on percentage of identity and/or percentage of similarity, with respect to polypeptide sequence, of individual sequences in the databases in comparison to CCP1 of Chlamydomonas reinhardtii, also as discussed above. For example, potential orthologs of CCP1 may be identified based on percentage of identity of an individual sequence in a database and CCP1 of Chlamydomonas reinhardtii of SEQ ID NO: 1 of at least 25%, e.g. at least 30%, at least 35%, at least 40%, at least 45%, at least 50%, at least 55%, at least 60%, at least 65%, at least 70%, at least 75%, at least 80%, at least 90%, or at least 95%, wherein the individual sequence is derived from eukaryotic algae. Also for example, potential orthologs of CCP1 may be identified based on percentage of similarity of an individual sequence in a database and CCP1 of Chlamydomonas reinhardtii of SEQ ID NO: 1 of at least 10%, e.g. at least 15%, at least 20%, at least 25%, at least 30%, at least 35%, at least 40%, at least 45%, at least 50%, at least 55%, at least 60%, at least 65%, at least 70%, at least 75%, at least 80%, at least 90%, or at least 95% , wherein the individual sequence is derived from eukaryotic algae. Also for example, potential orthologs of CCP1 may be identified based on both percentage of identity of at least 25%, e.g. at least 30%, at least 35%, at least 40%, at least 45%, at least 50%, at least 55%, at least 60%, at least 65%, at least 70%, at least 75%, at least 80%, at least 90%, or at least 95%, and percentage of similarity of at least 10%, e.g. at least 15%, at least 20%, at least 25%, at least 30%, at least 35%, at least 40%, at least 45%, at least 50%, at least 55%, at least 60%, at least 65%, at least 70%, at least 75%, at least 80%, at least 90%, or at least 95%, wherein the individual sequence is derived from eukaryotic algae.

Suitable mitochondrial transporter proteins also can be identified, for example, based on functional screens.

For example, some cyanobacterial bicarbonate transporters have previously been shown to functionally localize into the E. coli cytoplasmic membrane, as reported by Du et al. (2014), PLoS One 9, e115905. Expression of six particular cyanobacterial bicarbonate transporters in Escherichia coli using a mutant E. coli strain, termed EDCM636, that that is deficient in carbonic anhydrase activity and that is unable to grow on LB or M9 plates without supplementation with high levels of CO₂, restored growth of the E. coli mutant at atomospheric levels of CO₂, whereas expression of various others did not, as reported by Du et al. (2014). Function of CCP1 and potential orthologs thereof with respect to mitochondrial transport may be tested by an analogous approach, and corresponding functional screens developed, also based on restoring growth of this mutant E. coli strain that is deficient in carbonic anhydrase activity based on expressing CCP1 or potential orthologs thereof in the mutant E. coli strain.

Function of CCP1 and potential orthologs thereof with respect to mitochondrial transport also may be tested, and corresponding functional screens developed, based on the use of yeast modified to express CCP1 and potential orthologs thereof. Transport of bicarbonate from mitochondria of yeast so modified would indicate that these sequences also enable transport of bicarbonate in yeast.

Following identification of a mitochondrial transporter protein of a eukaryotic algae, modification of a land plant to express the mitochondrial transporter protein can be carried out by methods that are known in the art, as discussed in detail below.

As noted above, the mitochondrial transporter protein is expressed predominantly in seeds of the transgenic land plant. By this it is meant that the mitochondrial transporter protein is expressed at higher levels in cells of seeds of the transgenic land plant than in cells of stems, leaves, and roots of the transgenic land plant. For example, the mitochondrial transporter protein can be expressed in various tissues within seeds and at various stages of development of seeds. The expression can be absolutely specific to seeds, such that the mitochondrial transporter protein is only expressed in seeds, or can be preferentially in seeds, e.g. at rates that are higher by 2-fold, 5-fold, 10-fold, or more, in seeds relative to one or more other tissues of a plant, e.g. stems, leaves, and/or roots, among other tissues. This can be accomplished, for example, based on use of a seed-specific promoter for expression of a gene encoding the mitochondrial transporter protein, as discussed below. This also may be accomplished by other approaches, such as, for example, modifying stability of corresponding transcripts and/or the mitochondrial transporter itself, among others.

The transgenic land plant can be a transgenic land plant wherein the only heterologous algal protein that the transgenic land plant comprises is the mitochondrial transporter protein. As noted above, Atkinson et al. (2015) also discloses that expression of individual Ci transporters did not enhance Arabidopsis growth, and suggests that stacking of further components of carbon-concentrating mechanisms will probably be required to achieve a significant increase in photosynthetic efficiency in this species, albeit without having tested expression of CCP1 in particular. In contrast, without wishing to be bound by theory, it is believed that a transgenic land plant comprising a mitochondrial transporter protein of a eukaryotic algae, wherein the mitochondrial transporter protein of the eukaryotic algae is heterologous with respect to the transgenic land plant, the mitochondrial transporter protein corresponds to a sequence or ortholog of (a) CCP1 of Chlamydomonas reinhardtii of SEQ ID NO: 1, (b) a mitochondrial transporter protein of Chlorella sorokiniana of SEQ ID NO: 2, (c) a mitochondrial transporter protein of Chlorella variabilis of SEQ ID NO: 3, SEQ ID NO: 4, SEQ ID NO: 5, or SEQ ID NO: 6, (d) a mitochondrial transporter protein of Chondrus crispus of SEQ ID NO: 7, SEQ ID NO: 8, or SEQ ID NO: 9, (e) a mitochondrial transporter protein of Gonium pectorale of SEQ ID NO: 19, or SEQ ID NO: 20, or (f) a mitochondrial transporter protein of Volvox carteri of SEQ ID NO: 21, the mitochondrial transporter protein is localized to mitochondria of the transgenic land plant based on a mitochondrial targeting signal intrinsic to the mitochondrial transporter protein, and the mitochondrial transporter protein is expressed predominantly in seeds of the transgenic land plant, will achieve a significant increase in photosynthetic efficiency in the transgenic land plant without need for stacking of further components of carbon-concentrating mechanisms, and thus without expression of any other heterologous algal protein by the transgenic land plant. The corresponding transgenic land plant will provide advantages relative to plants that are modified to express multiple genes, for example in terms of simpler methods of making the transgenic land plant.

Considering the mitochondrial transporter protein in more detail, the mitochondrial transporter protein can correspond to a mitochondrial transporter protein selected from among specific polypeptide sequences of eukaryotic algae. As noted above, potential mitochondrial transporter proteins include CCP1 of Chlamydomonas reinhardtii of SEQ ID NO: 1. Potential mitochondrial transporter proteins also may be identified based on homology to CCP1. Exemplary mitochondrial transporter proteins identified this way include a mitochondrial transporter protein of a Chlorella sorokiniana of SEQ ID NO: 2. Such exemplary mitochondrial transporter proteins also include mitochondrial transporter proteins of a Chlorella variabilis of SEQ ID NO: 3, SEQ ID NO: 4, SEQ ID NO: 5, and SEQ ID NO: 6. Such exemplary mitochondrial transporter proteins also include mitochondrial transporter proteins of a Chondrus crispus of SEQ ID NO: 7, SEQ ID NO: 8, and SEQ ID NO: 9. Such exemplary mitochondrial transporter proteins also include mitochondrial transporter proteins of Gonium pectorale of SEQ ID NO: 19, or SEQ ID NO: 20. Such exemplary mitochondrial transporter proteins also include a mitochondrial transporter protein of Volvox carteri of SEQ ID NO: 21. Thus, for example, the mitochondrial transporter protein can correspond to a mitochondrial transporter protein selected from the group consisting of (a) CCP1 of Chlamydomonas reinhardtii of SEQ ID NO: 1, (b) a mitochondrial transporter protein of Chlorella sorokiniana of SEQ ID NO: 2, (c) a mitochondrial transporter protein of Chlorella variabilis of SEQ ID NO: 3, SEQ ID NO: 4, SEQ ID NO: 5, or SEQ ID NO: 6, (d) a mitochondrial transporter protein of Chondrus crispus of SEQ ID NO: 7, SEQ ID NO: 8, or SEQ ID NO: 9, (e) a mitochondrial transporter protein of Gonium pectorale of SEQ ID NO: 19, or SEQ ID NO: 20, and (f) a mitochondrial transporter protein of Volvox carteri of SEQ ID NO: 21.

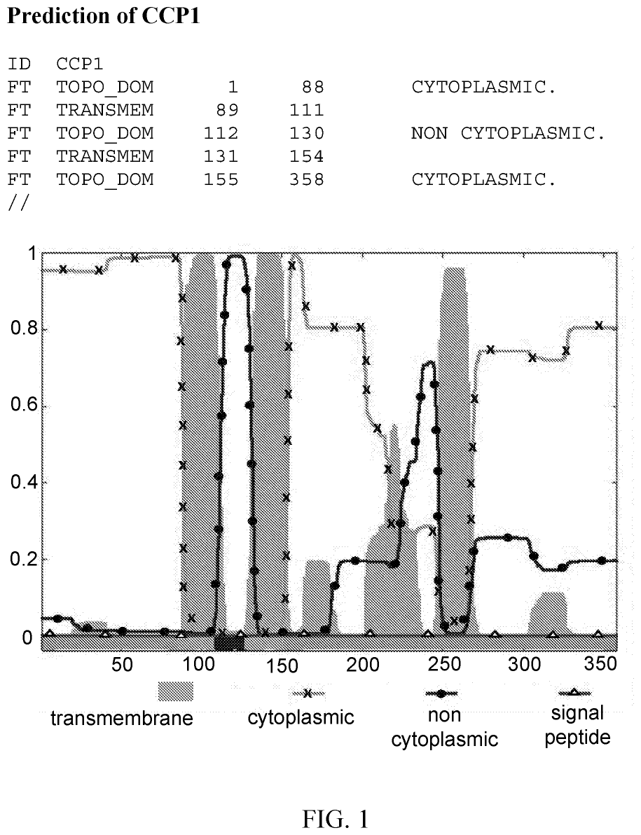

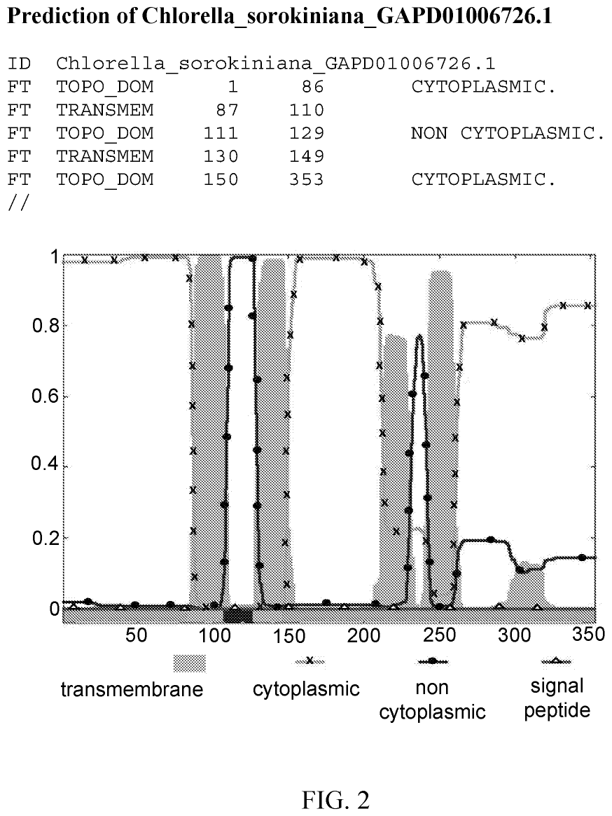

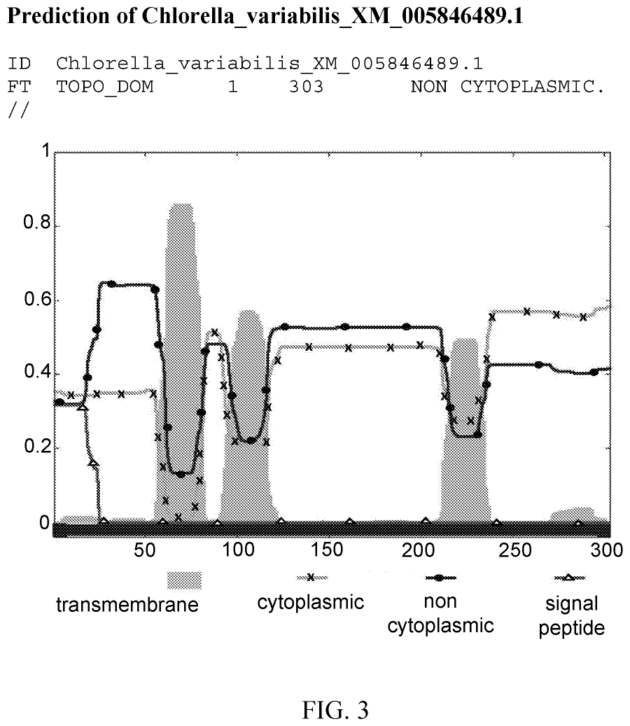

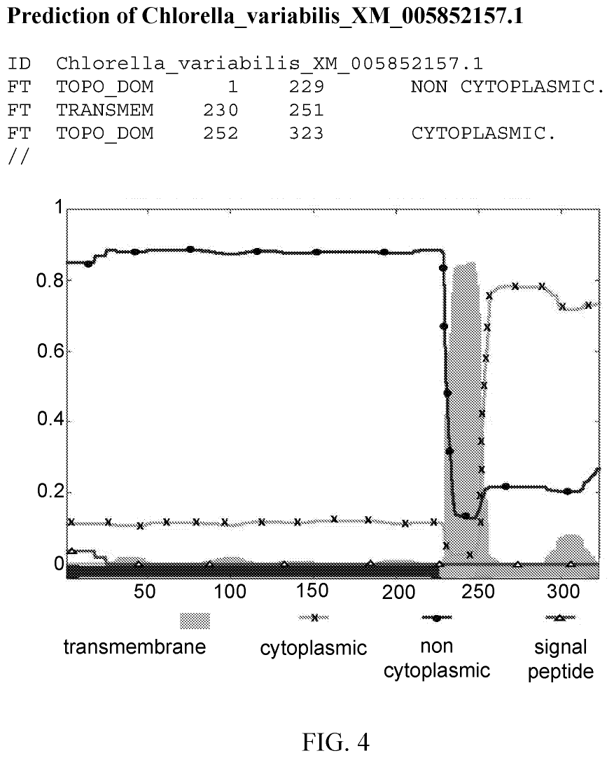

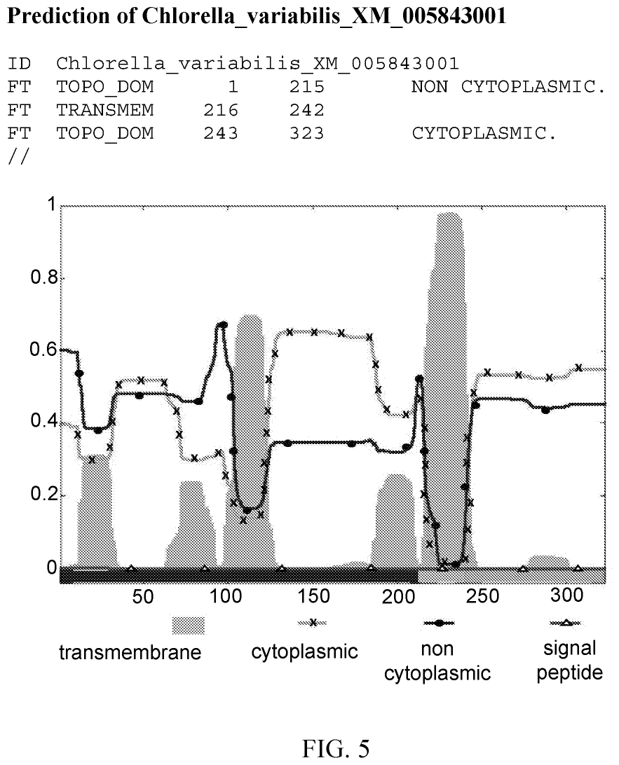

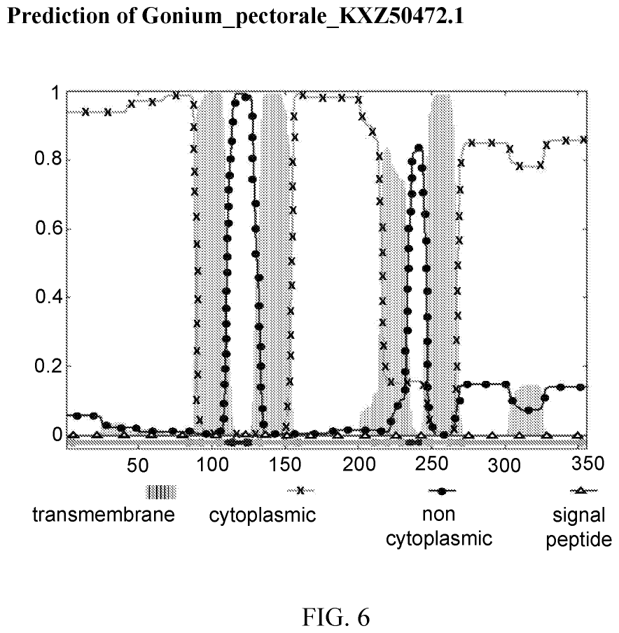

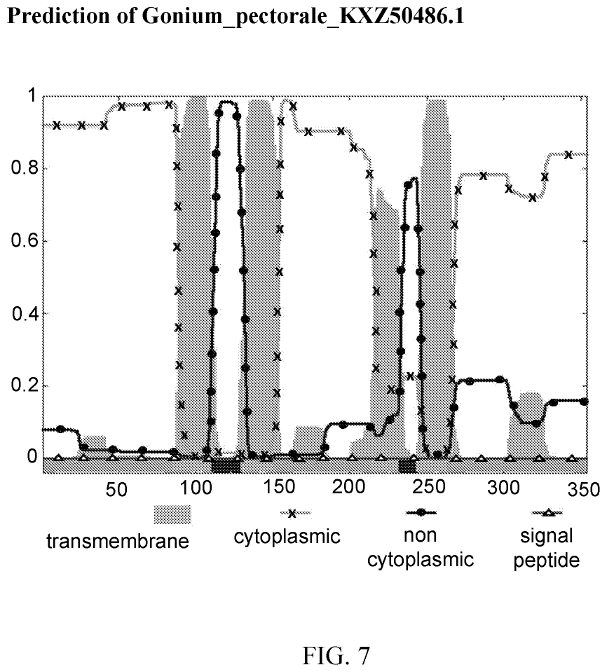

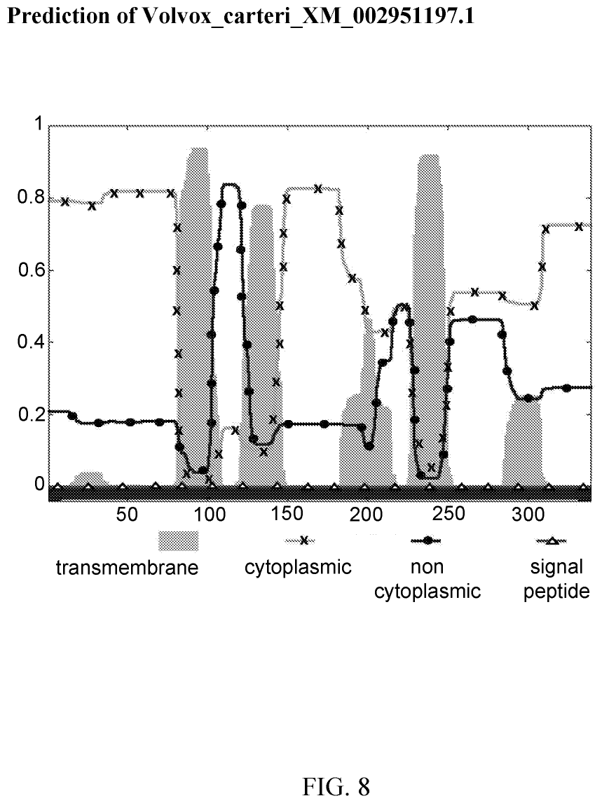

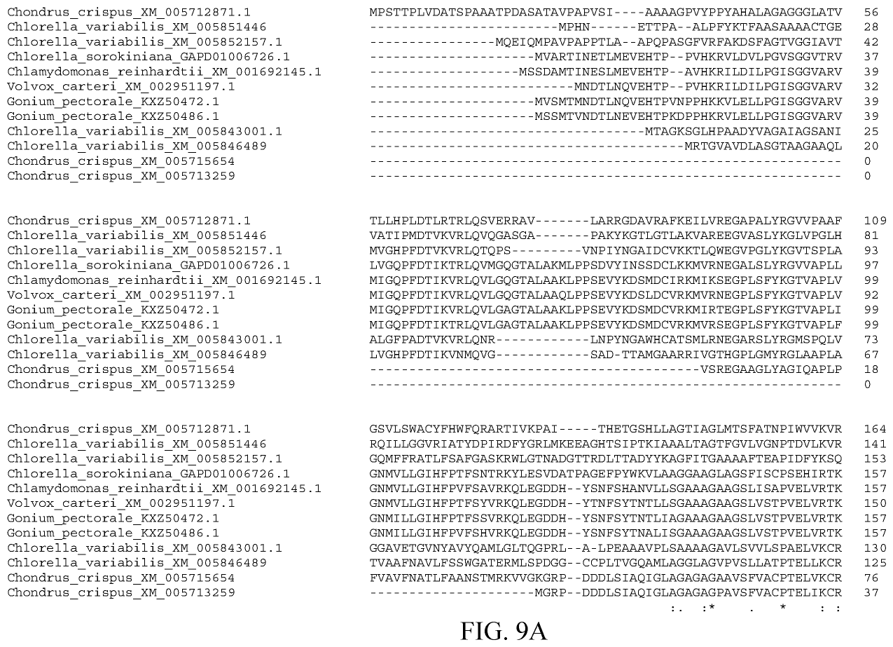

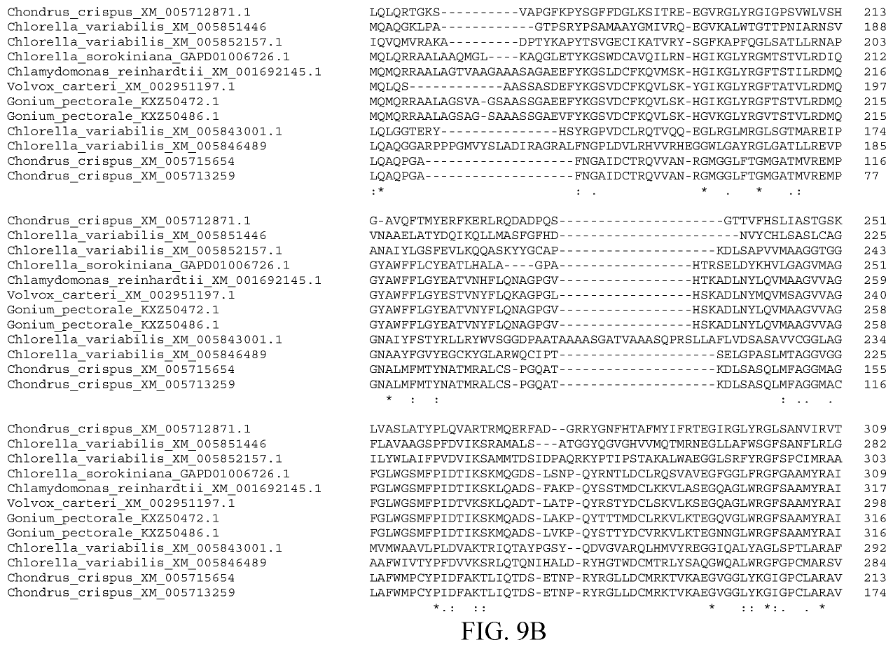

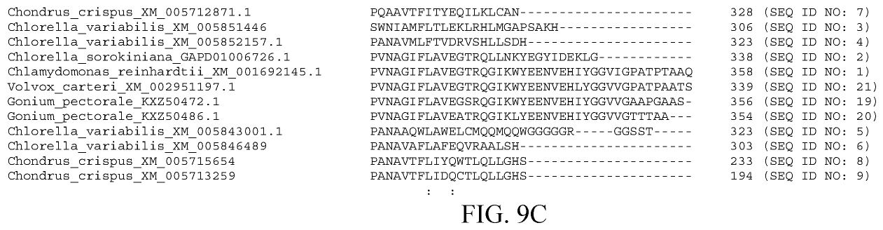

The mitochondrial transporter protein also can correspond to a mitochondrial transporter protein including specific structural features and characteristics shared among orthologs of CCP1 of Chlamydomonas reinhardtii of SEQ ID NO: 1. With reference to FIG. 1, FIG. 2, FIG. 3, FIG. 4, FIG. 5, FIG. 6, FIG. 7, FIG. 8, and FIG. 9A-C, such structural features and characteristics shared among the various orthologs of CCP1, namely the mitochondrial transporter proteins of SEQ ID NO: 2 to SEQ ID NO: 9 and SEQ ID NO: 19 to SEQ ID NO: 21, as identified based on multiple sequence alignment of CCP1 and the orthologs, include (i) (a) a proline residue at position 268, (b) an aspartate residue or glutamine residue at position 270, (c) a lysine residue or arginine residue at position 273, and (d) a serine residue or threonine residue at position 274, with numbering of positions relative to CCP1 of Chlamydomonas reinhardtii of SEQ ID NO: 1, and (ii) an overall identity of at least 15%. The noted amino acid residues, i.e. proline residue at position 268, aspartate residue or glutamine residue at position 270, lysine residue or arginine residue at position 273, and serine residue or threonine residue at position 274, with numbering of positions relative to CCP1 of Chlamydomonas reinhardtii of SEQ ID NO: 1, occur at or after the C-terminal portion of a potential transmembrane region of each of CCP1 and at least seven of the orthologs, namely that of Chlorella sorokiniana (GAPD01006726.1) of SEQ ID NO: 2, that of Chlorella variabilis (XM_005846489.1) of SEQ ID NO: 6, that of Chlorella variabilis (XM_005852157.1) of SEQ ID NO: 4, that of Chlorella variabilis (XM_005843001.1) of SEQ ID NO: 5, that of Gonium pectorale (KXZ50472.1) of SEQ ID NO: 19, that of Gonium pectorale (KXZ50486.1) of SEQ ID NO: 20, and that of Volvox carteri (XM_002951197.1) of SEQ ID NO: 21. Conservation of the noted amino acid residues, in combination with an overall identity of at least 15%, suggests a structure/function relationship shared among such mitochondrial transporter proteins. Thus, for example, the mitochondrial transporter protein can be an ortholog of CCP1 of Chlamydomonas reinhardtii of SEQ ID NO: 1 based on comprising: (i) (a) a proline residue at position 268, (b) an aspartate residue or glutamine residue at position 270, (c) a lysine residue or arginine residue at position 273, and (d) a serine residue or threonine residue at position 274, with numbering of positions relative to CCP1 of Chlamydomonas reinhardtii of SEQ ID NO: 1, and (ii) an overall identity of at least 15%.

The mitochondrial transporter protein also can correspond to a mitochondrial transporter protein including additional specific structural features and characteristics shared among orthologs of CCP1 of Chlamydomonas reinhardtii of SEQ ID NO: 1. For example, the mitochondrial transporter protein can be an ortholog of CCP1 of Chlamydomonas reinhardtii of SEQ ID NO: 1 based on comprising: (i) (a) a glycine residue at position 301, (b) a glycine residue at position 308, and (c) an arginine residue at position 315, with numbering of positions relative to CCP1 of Chlamydomonas reinhardtii of SEQ ID NO: 1, and (ii) an overall identity of at least 15%.

The mitochondrial transporter protein also can correspond to a mitochondrial transporter protein that does not only localize to mitochondria, but that also localizes to chloroplasts. As noted above, Atkinson et al. (2015) discloses that CCP1 and its homolog CCP2, which are characterized as putative Ci transporters previously reported to be in the chloroplast envelope, localized to mitochondria in both Chlamydomonas reinhardtii, as expressed naturally, and tobacco, when expressed heterologously. Without wishing to be bound by theory, it is believed that localization of CCP1 and orthologs thereof to mitochondria to a greater extent than to chloroplasts promotes enhanced yield. Thus, for example, the bicarbonate transporter protein can be localized to mitochondria of the transgenic land plant to a greater extent than to chloroplasts of the transgenic land plant by a factor of at least 2, at least 5, or at least 10.

The mitochondrial transporter protein also can correspond to a mitochondrial transporter protein that does not differ in any biologically significant way from a wild-type eukaryotic algal mitochondrial transporter protein. As noted above, the mitochondrial transporter protein is localized to mitochondria of the transgenic land plant based on a mitochondrial targeting signal intrinsic to the mitochondrial transporter protein, and this is in contrast, for example, to fusion of a heterologous mitochondrial targeting signal to a mitochondrial transporter protein that would not otherwise be targeted to mitochondria. In some examples, the mitochondrial transporter protein also does not include any other modifications that might result in the mitochondrial transporter protein differing in a biologically significant way from a wild-type eukaryotic algal mitochondrial transporter protein. Thus, for example the mitochondrial transporter protein can consist essentially of an amino acid sequence that is identical to that of a wild-type eukaryotic algal mitochondrial transporter protein. The corresponding transgenic land plant will provide advantages, e.g. in terms of simpler methods of making the transgenic land plant.

The transgenic land plant can further comprise a heterologous polynucleotide, wherein the mitochondrial transporter protein is encoded by the heterologous polynucleotide. For example, the heterologous polynucleotide can comprise a heterologous promoter. Also for example, the heterologous promoter can be a seed-specific promoter. Also for example, the heterologous polynucleotide can be integrated into genomic DNA of the transgenic land plant. These exemplary features of the heterologous polynucleotide, and others, are discussed in detail below.

The transgenic land plant also can be a transgenic land plant that expresses eukaryotic algal mitochondrial transporter genes in both a seed-specific and a constitutive manner, wherein the eukaryotic algal mitochondrial transporter genes may be the same or different genes, from the same algal species or from different algal species. Without wishing to be bound by theory, it is believed that constitutive expression results in much higher numbers of pods, and that seed-specific expression can supply the carbon needed to fill seeds to a full size, and that thus the yield should be higher. Accordingly, is some examples the transgenic land plant (i) expresses the mitochondrial transporter protein in a seed-specific manner, and (ii) expresses another mitochondrial transporter protein constitutively, the other mitochondrial transporter protein also corresponding to a sequence or ortholog of (a) CCP1 of Chlamydomonas reinhardtii of SEQ ID NO: 1, (b) a mitochondrial transporter protein of Chlorella sorokiniana of SEQ ID NO: 2, (c) a mitochondrial transporter protein of Chlorella variabilis of SEQ ID NO: 3, SEQ ID NO: 4, SEQ ID NO: 5, or SEQ ID NO: 6, (d) a mitochondrial transporter protein of Chondrus crispus of SEQ ID NO: 7, SEQ ID NO: 8, or SEQ ID NO: 9, (e) a mitochondrial transporter protein of Gonium pectorale of SEQ ID NO: 19, or SEQ ID NO: 20, or (f) a mitochondrial transporter protein of Volvox carteri of SEQ ID NO: 21.

The transgenic land plant can have a CO₂ assimilation rate that is higher than for a corresponding reference land plant not comprising the mitochondrial transporter protein. For example, the transgenic land plant can have a CO₂ assimilation rate that is at least 5% higher, at least 10% higher, at least 20% higher, or at least 40% higher, than for a corresponding reference land plant not comprising the mitochondrial transporter protein.

The transgenic land plant also can have a transpiration rate that is lower than for a corresponding reference land plant not comprising the mitochondrial transporter protein. For example, the transgenic land plant can have transpiration rate that is at least 5% lower, at least 10% lower, at least 20% lower, or at least 40% lower, than for a corresponding reference land plant not comprising the mitochondrial transporter protein.

The transgenic land plant also can have a number of branches of the main stem that is higher than for a corresponding reference land plant not comprising the mitochondrial transporter protein. For example, the transgenic land plant can have a number of branches of the main stem that is at least 5% higher, at least 10% higher, at least 20% higher, at least 40% higher, at least 60% higher, or at least 80% higher, than for a corresponding reference land plant not comprising the mitochondrial transporter protein.

The transgenic land plant also can have a number of tillers, flowers (inflorescences), buds, or panicles that is higher than for a corresponding reference land plant not comprising the mitochondrial transporter protein. For example, the transgenic land plant can have a number of tillers, flowers (inflorescences), buds or panicles of the main stem that is at least 5% higher, at least 10% higher, at least 20% higher, at least 40% higher, at least 60% higher, or at least 80% higher, than for a corresponding reference land plant not comprising the mitochondrial transporter protein.

The transgenic land plant also can have a number of seed pods that is higher than for a corresponding reference land plant not comprising the mitochondrial transporter protein. For example, the transgenic land plant can have a number of seed pods that is at least 5% higher, at least 10% higher, at least 20% higher, at least 40% higher, at least 60% higher, or at least 80% higher, than for a corresponding reference land plant not comprising the mitochondrial transporter protein.

The transgenic land plant also can have a seed yield that is higher than for a corresponding reference land plant not comprising the mitochondrial transporter protein. For example, the transgenic land plant can have a seed yield that is at least 5% higher, at least 10% higher, at least 20% higher, at least 40% higher, at least 60% higher, or at least 80% higher, than for a corresponding reference land plant not comprising the mitochondrial transporter protein.

As noted above, following identification of a mitochondrial transporter protein of a eukaryotic algae, modification of a land plant to express the mitochondrial transporter protein can be carried out by methods that are known in the art, for example as follows.

DNA constructs useful in the methods described herein include transformation vectors capable of introducing transgenes into land plants. As used herein, “transgenic” refers to an organism in which a nucleic acid fragment containing a heterologous nucleotide sequence has been introduced. The transgenes in the transgenic organism are preferably stable and inheritable. The heterologous nucleic acid fragment may or may not be integrated into the host genome.

Several plant transformation vector options are available, including those described in Gene Transfer to Plants, 1995, Potrykus et al., eds., Springer-Verlag Berlin Heidelberg New York, Transgenic Plants: A Production System for Industrial and Pharmaceutical Proteins, 1996, Owen et al., eds., John Wiley & Sons Ltd. England, and Methods in Plant Molecular Biology: A Laboratory Course Manual, 1995, Maliga et al., eds., Cold Spring Laboratory Press, New York. Plant transformation vectors generally include one or more coding sequences of interest under the transcriptional control of 5′ and 3′ regulatory sequences, including a promoter, a transcription termination and/or polyadenylation signal, and a selectable or screenable marker gene.

Many vectors are available for transformation using Agrobacterium tumefaciens. These typically carry at least one T-DNA sequence and include vectors such as pBIN19. Typical vectors suitable for Agrobacterium transformation include the binary vectors pCIB200 and pCIB2001, as well as the binary vector pCIB 10 and hygromycin selection derivatives thereof (See, for example, U.S. Pat. No. 5,639,949).

Transformation without the use of Agrobacterium tumefaciens circumvents the requirement for T-DNA sequences in the chosen transformation vector and consequently vectors lacking these sequences are utilized in addition to vectors such as the ones described above which contain T-DNA sequences. The choice of vector for transformation techniques that do not rely on Agrobacterium depends largely on the preferred selection for the species being transformed. Typical vectors suitable for non-Agrobacterium transformation include pCIB3064, pSOG 19, and pSOG35. (See, for example, U.S. Pat. No 5,639,949). Alternatively, DNA fragments containing the transgene and the necessary regulatory elements for expression of the transgene can be excised from a plasmid and delivered to the plant cell using microprojectile bombardment-mediated methods.

Zinc-finger nucleases (ZFNs) are also useful for practicing the invention in that they allow double strand DNA cleavage at specific sites in plant chromosomes such that targeted gene insertion or deletion can be performed (Shukla et al., 2009, Nature 459: 437-441; Townsend et al., 2009, Nature 459: 442-445).

The CRISPR/Cas9 system (Sander, J. D. and Joung, J. K., Nature Biotechnology, published online March 2, 2014; doi;10.1038/nbt.2842) is particularly useful for editing plant genomes to modulate the expression of homologous genes encoding enzymes. Several examples of the use of this technology to edit the genomes of plants have now been reported (Belhaj et al. Plant Methods 2013, 9:39).

Transformation protocols as well as protocols for introducing nucleotide sequences into plants may vary depending on the type of plant or plant cell targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606), Agrobacterium-mediated transformation (Townsend et al., U.S. Pat. No. 5,563,055; Zhao et al. WO US98/01268), direct gene transfer (Paszkowski et al. (1984) EMBO J. 3:2717-2722), and ballistic particle acceleration (see, for example, Sanford et al., U.S. Pat. No. 4,945,050; Tomes et al. (1995) Plant Cell, Tissue, and Organ Culture: Fundamental Methods, ed. Gamborg and Phillips (Springer-Verlag, Berlin); and McCabe et al. Biotechnology 6:923-926 (1988)). Also see Weissinger et al. Ann. Rev. Genet. 22:421-477 (1988); Sanford et al. Particulate Science and Technology 5:27-37 (1987) (onion); Christou et al. Plant Physiol. 87:671-674 (1988) (soybean); McCabe et al. (1988) BioTechnology 6:923-926 (soybean); Finer and McMullen In Vitro Cell Dev. Biol. 27P:175-182 (1991) (soybean); Singh et al. Theor. Appl. Genet. 96:319-324 (1998)(soybean); Dafta et al. (1990) Biotechnology 8:736-740 (rice); Klein et al. Proc. Natl. Acad. Sci. USA 85:4305-4309 (1988) (maize); Klein et al. Biotechnology 6:559-563 (1988) (maize); Tomes, U.S. Pat. No. 5,240,855; Buising et al., U.S. Pat. Nos. 5,322,783 and 5,324,646; Tomes et al. (1995) in Plant Cell, Tissue, and Organ Culture: Fundamental Methods, ed. Gamborg (Springer-Verlag, Berlin) (maize); Klein et al. Plant Physiol. 91:440-444 (1988) (maize); Fromm et al. Biotechnology 8:833-839 (1990) (maize); Hooykaas-Van Slogteren et al. Nature 311:763-764 (1984); Bowen et al., U.S. Pat. No. 5,736,369 (cereals); Bytebier et al. Proc. Natl. Acad. Sci. USA 84:5345-5349 (1987) (Liliaceae); De Wet et al. in The Experimental Manipulation of Ovule Tissues, ed. Chapman et al. (Longman, N.Y.), pp. 197-209 (1985) (pollen); Kaeppler et al. Plant Cell Reports 9:415-418 (1990) and Kaeppler et al. Theor. Appl. Genet. 84:560-566 (1992) (whisker-mediated transformation); D'Halluin et al. Plant Cell 4:1495-1505 (1992) (electroporation); Li et al. Plant Cell Reports 12:250-255 (1993) and Christou and Ford Annals of Botany 75:407-413 (1995) (rice); Osjoda et al. Nature Biotechnology 14:745-750 (1996) (maize via Agrobacterium tumefaciens); all of which are herein incorporated by reference in their entirety. Methods for transforming plant protoplasts are available including transformation using polyethylene glycol (PEG) , electroporation, and calcium phosphate precipitation (see for example Potrykus et al., 1985, Mol. Gen. Genet., 199, 183-188; Potrykus et al., 1985, Plant Molecular Biology Reporter, 3, 117-128), Methods for plant regeneration from protoplasts have also been described [Evans et al., in Handbook of Plant Cell Culture, Vol 1, (Macmillan Publishing Co., New York, 1983); Vasil, I K in Cell Culture and Somatic Cell Genetics (Academic, Orlando, 1984)].

Recombinase technologies which are useful for producing the disclosed transgenic plants include the cre-lox, FLP/FRT and Gin systems. Methods by which these technologies can be used for the purpose described herein are described for example in (U.S. Pat. No. 5,527,695; Dale and Ow, 1991, Proc. Natl. Acad. Sci. USA 88: 10558-10562; Medberry et al., 1995, Nucleic Acids Res. 23: 485-490).

Transformation protocols as well as protocols for introducing nucleotide sequences into plants may vary depending on the type of plant or plant cell, i.e., monocot or dicot, targeted for transformation.

Suitable methods of introducing nucleotide sequences into plant cells and subsequent insertion into the plant genome are described in US 2010/0229256 A1 to Somleva & Ali and US 2012/0060413 to Somleva et al.

The transformed cells are grown into plants in accordance with conventional techniques. See, for example, McCormick et al., 1986, Plant Cell Rep. 5: 81-84. These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

Procedures for in planta transformation can be simple. Tissue culture manipulations and possible somaclonal variations are avoided and only a short time is required to obtain transgenic plants. However, the frequency of transformants in the progeny of such inoculated plants is relatively low and variable. At present, there are very few species that can be routinely transformed in the absence of a tissue culture-based regeneration system. Stable Arabidopsis transformants can be obtained by several in planta methods including vacuum infiltration (Clough & Bent, 1998, The Plant 1 16: 735-743), transformation of germinating seeds (Feldmann & Marks, 1987, Mol. Gen. Genet. 208: 1-9), floral dip (Clough and Bent, 1998, Plant J. 16: 735-743), and floral spray (Chung et al., 2000, Transgenic Res. 9: 471-476). Other plants that have successfully been transformed by in planta methods include rapeseed and radish (vacuum infiltration, Ian and Hong, 2001, Transgenic Res., 10: 363-371; Desfeux et al., 2000, Plant Physiol. 123: 895-904), Medicago truncatula (vacuum infiltration, Trieu et al., 2000, Plant J. 22: 531-541), camelina (floral dip, WO/2009/117555 to Nguyen et al.), and wheat (floral dip, Zale et al., 2009, Plant Cell Rep. 28: 903-913). In planta methods have also been used for transformation of germ cells in maize (pollen, Wang et al. 2001, Acta Botanica Sin., 43, 275-279; Zhang et al., 2005, Euphytica, 144, 11-22; pistils, Chumakov et al. 2006, Russian I Genetics, 42, 893-897; Mamontova et al. 2010, Russian J. Genetics, 46, 501-504) and Sorghum (pollen, Wang et al. 2007, Biotechnol. Appl. Biochem., 48, 79-83).

Following transformation by any one of the methods described above, the following procedures can be used to obtain a transformed plant expressing the transgenes: select the plant cells that have been transformed on a selective medium; regenerate the plant cells that have been transformed to produce differentiated plants; select transformed plants expressing the transgene producing the desired level of desired polypeptide(s) in the desired tissue and cellular location.

The cells that have been transformed may be grown into plants in accordance with conventional techniques. See, for example, McCormick et al. Plant Cell Reports 5:81-84(1986). These plants may then be grown, and either pollinated with the same transformed variety or different varieties, and the resulting hybrid having constitutive expression of the desired phenotypic characteristic identified. Two or more generations may be grown to ensure that expression of the desired phenotypic characteristic is stably maintained and inherited and then seeds harvested to ensure constitutive expression of the desired phenotypic characteristic has been achieved.

Transgenic plants can be produced using conventional techniques to express any genes of interest in plants or plant cells (Methods in Molecular Biology, 2005, vol. 286, Transgenic Plants: Methods and Protocols, Pena L., ed., Humana Press, Inc. Totowa, N.J.; Shyamkumar Barampuram and Zhanyuan J. Zhang, Recent Advances in Plant Transformation, in James A. Birchler (ed.), Plant Chromosome Engineering: Methods and Protocols, Methods in Molecular Biology, vol. 701, Springer Science+Business Media). Typically, gene transfer, or transformation, is carried out using explants capable of regeneration to produce complete, fertile plants. Generally, a DNA or an RNA molecule to be introduced into the organism is part of a transformation vector. A large number of such vector systems known in the art may be used, such as plasmids. The components of the expression system can be modified, e.g., to increase expression of the introduced nucleic acids. For example, truncated sequences, nucleotide substitutions or other modifications may be employed. Expression systems known in the art may be used to transform virtually any plant cell under suitable conditions. A transgene comprising a DNA molecule encoding a gene of interest is preferably stably transformed and integrated into the genome of the host cells. Transformed cells are preferably regenerated into whole fertile plants. Detailed description of transformation techniques are within the knowledge of those skilled in the art.

Plant promoters can be selected to control the expression of the transgene in different plant tissues or organelles for all of which methods are known to those skilled in the art (Gasser & Fraley, 1989, Science 244: 1293-1299). In one embodiment, promoters are selected from those of eukaryotic or synthetic origin that are known to yield high levels of expression in plants and algae. In a preferred embodiment, promoters are selected from those that are known to provide high levels of expression in monocots.

Chemical-regulated promoters can be used to modulate the expression of a gene in a plant through the application of an exogenous chemical regulator.

Constitutive promoters include, for example, the core promoter of the Rsyn7 promoter and other constitutive promoters disclosed in WO 99/43838 and U.S. Pat. No. 6,072,050, the core CaMV 35S promoter (Odell et al., 1985, Nature 313: 810-812), rice actin (McElroy et al., 1990, Plant Cell 2: 163-171), ubiquitin (Christensen et al., 1989, Plant Mol. Biol. 12: 619-632; Christensen et al., 1992, Plant Mol. Biol. 18: 675-689), pEMU (Last et al., 1991, Theor. Appl. Genet. 81: 581-588), MAS (Velten et al., 1984, EMBO J. 3: 2723-2730), and ALS promoter (U.S. Pat. No 5,659,026). Other constitutive promoters are described in U.S. Pat. Nos 5,608,149; 5,608,144; 5,604,121; 5,569,597; 5,466,785; 5,399,680; 5,268,463; and 5,608,142.

“Tissue-preferred” promoters can be used to target gene expression within a particular tissue. Tissue-preferred promoters include those described by Van Ex et al., 2009, Plant Cell Rep. 28: 1509-1520; Yamamoto et al., 1997, Plant J. 12: 255-265; Kawamata et al., 1997, Plant Cell Physiol. 38: 792-803; Hansen et al., 1997, Mol. Gen. Genet. 254: 337-343; Russell et al., 199), Transgenic Res. 6: 157-168; Rinehart et al., 1996, Plant Physiol. 112: 1331-1341; Van Camp et al., 1996, Plant Physiol. 112: 525-535; Canevascini et al., 1996, Plant Physiol. 112: 513-524; Yamamoto et al., 1994, Plant Cell Physiol. 35: 773-778; Lam, 1994, Results Probl. Cell Differ. 20: 181-196, Orozco et al., 1993, Plant Mol. Biol. 23: 1129-1138; Matsuoka et al., 1993, Proc. Natl. Acad. Sci. USA 90: 9586-9590, and Guevara-Garcia et al., 1993, Plant J. 4: 495-505. Such promoters can be modified, if necessary, for weak expression.

Seed-specific promoters can be used to target gene expression to seeds in particular. Seed-specific promoters include promoters that are expressed in various tissues within seeds and at various stages of development of seeds. Seed-specific promoters can be absolutely specific to seeds, such that the promoters are only expressed in seeds, or can be expressed preferentially in seeds, e.g. at rates that are higher by 2-fold, 5-fold, 10-fold, or more, in seeds relative to one or more other tissues of a plant, e.g. stems, leaves, and/or roots, among other tissues. Seed-specific promoters include, for example, seed-specific promoters of dicots and seed-specific promoters of monocots, among others. For dicots, seed-specific promoters include, but are not limited to, bean β-phaseolin, napin, β-conglycinin, soybean oleosin 1, Arabidopsis thaliana sucrose synthase, flax conlinin soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, and globulin 1.

Certain embodiments use transgenic plants or plant cells having multi-gene expression constructs harboring more than one promoter. The promoters can be the same or different.

Any of the described promoters can be used to control the expression of one or more of the genes of the invention, their homologs and/or orthologs as well as any other genes of interest in a defined spatiotemporal manner.

Nucleic acid sequences intended for expression in transgenic plants are first assembled in expression cassettes behind a suitable promoter active in plants. The expression cassettes may also include any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be transferred to the plant transformation vectors described infra. The following is a description of various components of typical expression cassettes.

A variety of transcriptional terminators are available for use in expression cassettes. These are responsible for the termination of transcription beyond the transgene and the correct polyadenylation of the transcripts. Appropriate transcriptional terminators are those that are known to function in plants and include the CaMV 35S terminator, the tml terminator, the nopaline synthase terminator and the pea rbcS E9 terminator. These are used in both monocotyledonous and dicotyledonous plants.

The coding sequence of the selected gene may be genetically engineered by altering the coding sequence for optimal expression in the crop species of interest. Methods for modifying coding sequences to achieve optimal expression in a particular crop species are well known (Perlak et al., 1991, Proc. Natl. Acad. Sci. USA 88: 3324 and Koziel et al., 1993, Biotechnology 11: 194-200).

A recombinant DNA construct including a plant-expressible gene or other DNA of interest is inserted into the genome of a plant by a suitable method. Suitable methods include, for example, Agrobacterium tumefaciens-mediated DNA transfer, direct DNA transfer, liposome-mediated DNA transfer, electroporation, co-cultivation, diffusion, particle bombardment, microinjection, gene gun, calcium phosphate coprecipitation, viral vectors, and other techniques. Suitable plant transformation vectors include those derived from a Ti plasmid of Agrobacterium tumefaciens. In addition to plant transformation vectors derived from the Ti or root-inducing (Ri) plasmids of Agrobacterium, alternative methods can be used to insert DNA constructs into plant cells. A transgenic plant can be produced by selection of transformed seeds or by selection of transformed plant cells and subsequent regeneration. Individual plants within a population of transgenic plants that express a recombinant gene(s) may have different levels of gene expression. The variable gene expression is due to multiple factors including multiple copies of the recombinant gene, chromatin effects, and gene suppression. Accordingly, a phenotype of the transgenic plant may be measured as a percentage of individual plants within a population. The yield of a plant can be measured simply by weighing. The yield of seed from a plant can also be determined by weighing.

Genetic constructs may encode a selectable marker to enable selection of transformation events. There are many methods that have been described for the selection of transformed plants [for review see (Miki et al., Journal of Biotechnology, 2004, 107, 193-232) and references incorporated within]. Selectable marker genes that have been used extensively in plants include the neomycin phosphotransferase gene nptll (U.S. Pat. Nos. 5,034,322, U.S. 5,530,196), hygromycin resistance gene (U.S. Pat. No. 5,668,298, Waldron et al., (1985), Plant Mol Biol, 5:103-108; Zhijian et al., (1995), Plant Sci, 108:219-227), the bar gene encoding resistance to phosphinothricin (U.S. Pat. No. 5,276,268), the expression of aminoglycoside 3″-adenyltransferase (aadA) to confer spectinomycin resistance (U.S. Pat. No. 5,073,675), the use of inhibition resistant 5-enolpyruvyl-3-phosphoshikimate synthetase (U.S. Pat. No. 4,535,060) and methods for producing glyphosate tolerant plants (U.S. Pat. No. 5,463,175; U.S. Pat. No. 7,045,684). Other suitable selectable markers include, but are not limited to, genes encoding resistance to chloramphenicol (Herrera Estrella et al., (1983), EMBO J, 2:987-992), methotrexate (Herrera Estrella et al., (1983), Nature, 303:209-213; Meijer et al, (1991), Plant Mol Biol, 16:807-820); streptomycin (Jones et al., (1987), Mol Gen Genet, 210:86-91); bleomycin (Hille et al., (1990), Plant Mol Biol, 7:171-176) ; sulfonamide (Guerineau et al., (1990), Plant Mol Biol, 15:127-136); bromoxynil (Stalker et al., (1988), Science, 242:419-423); glyphosate (Shaw et al., (1986), Science, 233:478-481); phosphinothricin (DeBlock et al., (1987), EMBO J, 6:2513-2518).

Methods of plant selection that do not use antibiotics or herbicides as a selective agent have been previously described and include expression of glucosamine-6-phosphate deaminase to inactive glucosamine in plant selection medium (U.S. Pat. No. 6,444,878) and a positive/negative system that utilizes D-amino acids (Erikson et al., Nat Biotechnol, 2004, 22, 455-8). European Patent Publication No. EP 0 530 129 A1 describes a positive selection system which enables the transformed plants to outgrow the non-transformed lines by expressing a transgene encoding an enzyme that activates an inactive compound added to the growth media. U.S. Pat. No. 5,767,378 describes the use of mannose or xylose for the positive selection of transgenic plants.

Methods for positive selection using sorbitol dehydrogenase to convert sorbitol to fructose for plant growth have also been described (WO 2010/102293). Screenable marker genes include the beta-glucuronidase gene (Jefferson et al., 1987, EMBO 1 6: 3901-3907; U.S. Pat. No. 5,268,463) and native or modified green fluorescent protein gene (Cubitt et al., 1995, Trends Biochem. Sci. 20: 448-455; Pan et al., 1996, Plant Physiol. 112: 893-900).

Transformation events can also be selected through visualization of fluorescent proteins such as the fluorescent proteins from the nonbioluminescent Anthozoa species which include DsRed, a red fluorescent protein from the Discosoma genus of coral (Matz et al. (1999), Nat Biotechnol 17: 969-73). An improved version of the DsRed protein has been developed (Bevis and Glick (2002), Nat Biotech 20: 83-87) for reducing aggregation of the protein.

Visual selection can also be performed with the yellow fluorescent proteins (YFP) including the variant with accelerated maturation of the signal (Nagai, T. et al. (2002), Nat Biotech 20: 87-90), the blue fluorescent protein, the cyan fluorescent protein, and the green fluorescent protein (Sheen et al. (1995), Plant J 8: 777-84; Davis and Vierstra (1998), Plant Molecular Biology 36: 521-528). A summary of fluorescent proteins can be found in Tzfira et al. (Tzfira et al. (2005), Plant Molecular Biology 57: 503-516) and Verkhusha and Lukyanov (Verkhusha, V. V. and K. A. Lukyanov (2004),Nat Biotech 22: 289-296) whose references are incorporated in entirety. Improved versions of many of the fluorescent proteins have been made for various applications. Use of the improved versions of these proteins or the use of combinations of these proteins for selection of transformants will be obvious to those skilled in the art.

The plants modified for enhanced yield may have stacked input traits that include herbicide resistance and insect tolerance, for example a plant that is tolerant to the herbicide glyphosate and that produces the Bacillus thuringiensis (BT) toxin. Glyphosate is a herbicide that prevents the production of aromatic amino acids in plants by inhibiting the enzyme 5-enolpyruvylshikimate-3-phosphate synthase (EPSP synthase). The overexpression of EPSP synthase in a crop of interest allows the application of glyphosate as a weed killer without killing the modified plant (Suh, et al., J. M Plant Mol. Biol. 1993, 22, 195-205). BT toxin is a protein that is lethal to many insects providing the plant that produces it protection against pests (Barton, et al. Plant Physiol. 1987, 85, 1103-1109). Other useful herbicide tolerance traits include but are not limited to tolerance to Dicamba by expression of the dicamba monoxygenase gene (Behrens et al, 2007, Science, 316, 1185), tolerance to 2,4-D and 2,4-D choline by expression of a bacterial aad-1 gene that encodes for an aryloxyalkanoate dioxygenase enzyme (Wright et al., Proceedings of the National Academy of Sciences, 2010, 107, 20240), glufosinate tolerance by expression of the bialophos resistance gene (bar) or the pat gene encoding the enzyme phosphinotricin acetyl transferase (Droge et al., Planta, 1992, 187, 142), as well as genes encoding a modified 4-hydroxyphenylpyruvate dioxygenase (HPPD) that provides tolerance to the herbicides mesotrione, isoxaflutole, and tembotrione. (Siehl et al., Plant Physiol, 2014, 166, 1162).

The transgenic land plant that comprises a mitochondrial transporter protein of a eukaryotic algae, as disclosed, can be modified to further enhance yield.

One approach for further enhanced yield comprises modifying the transgenic land plant for reduced expression of cell wall invertase inhibitor (also termed CCWI). It is believed that expression of a novel class of cell wall invertase inhibitors is upregulated in plants modified to express CCP1 of Chlamydomonas reinhardtii and/or mitochondrial transporter proteins of eukaryotic algae that are orthologs of CCP1, and that downregulating cell wall invertase inhibitor genes in plants modified to express CCP1 of Chlamydomonas reinhardtii and/or mitochondrial transporter proteins of eukaryotic algae that are orthologs of CCP1 would result in further enhanced yield, as discussed below.

Cell wall invertase inhibitors of plants such as tomato and rice are known in the art, as taught for example by Wang et al. (2008), Nature Genetics 40(11):1370-1374, and Jin et al. (2009), Plant Cell 21(7):2072-2089, and can be identified in other plants, for example based on homology, in accordance with methods known in the art.

Modifying the transgenic land plant for reduced expression of cell wall invertase inhibitor can be accomplished, for example, by expressing a suppressor of an endogenous cell wall invertase inhibitor of the transgenic land plant, for example by antisense RNA or RNA interference, in accordance with methods known in the art. Such modification also can be accomplished, for example, by expressing a modified cell wall invertase inhibitor in place of an endogenous cell wall invertase inhibitor of the transgenic land plant, for example by CRISPR-associated protein 9 modification of a gene encoding the endogenous cell wall invertase inhibitor, also in accordance with methods known in the art.

Accordingly, in some examples the transgenic land plant is modified to express (i) a suppressor of an endogenous cell wall invertase inhibitor of the transgenic land plant or (ii) a modified cell wall invertase inhibitor in place of an endogenous cell wall invertase inhibitor of the transgenic land plant. In some of these examples relating to a suppressor of the endogenous cell wall invertase inhibitor, the suppressor is (i) an antisense RNA complementary to messenger RNA of the endogenous cell wall invertase inhibitor or (ii) an RNA interference nucleic acid that reduces expression of messenger RNA of the endogenous cell wall invertase inhibitor. Also, in some of these examples relating to a modified cell wall invertase inhibitor, the modified cell wall invertase inhibitor has been modified by transforming the transgenic land plant with a nucleotide sequence encoding CRISPR-associated protein 9 under the control of a promoter and with a nucleotide sequence encoding a single guide RNA under the control of a promoter, wherein the single guide RNA comprises 19 to 22 nucleotides and is fully homologous to a region of a gene encoding the endogenous cell wall invertase inhibitor.

Another approach for further enhanced yield comprises modifying the transgenic land plant to express carbonic anhydrase targeted to mitochondria. As noted above, the carbon-concentrating mechanism of eukaryotic algae includes expression of a and carbonic anhydrases for concentration of bicarbonate in chloroplast stroma. More specifically, carbonic anhydrases catalyze reversible hydration of CO₂ to bicarbonate and play a central role in controlling pH balance and inorganic carbon sequestration and flux. It is believed that expressing carbonic anhydrase targeted to mitochondria in plants modified to express CCP1 of Chlamydomonas reinhardtii and/or mitochondrial transporter proteins of eukaryotic algae that are orthologs of CCP1 may further enhance availability of bicarbonate or other metabolites for CCP1 and/or the mitochondrial transporter proteins of eukaryotic algae that are orthologs of CCP1 to export to cytosol of cells.

Carbonic anhydrase of plants such as rice, maize, soybean, tomato, barley, cucumber, alfalfa, bean, pea, pear, almond, mung bean, tobacco, cotton, aspen, and Arabidopsis are known in the art, as taught for example by Schroeder, U.S. Pat. No. 8,916,745 and references cited therein, and can be identified in other plants, for example based on homology, in accordance with methods known in the art.

Modifying the transgenic land plant to express carbonic anhydrase targeted to mitochondria can be carried out by methods that are known in the art, as discussed above. The carbonic anhydrase can be, for example, a carbonic anhydrase that is targeted to mitochondria based on including an endogenous mitochondrial targeting signal, or a carbonic anhydrase that is targeted to mitochondria based on having been engineered to include a mitochondrial targeting signal. The carbonic anhydrase also can be, for example, a plant carbonic anhydrase. The plant carbonic anhydrase can be, for example, a carbonic anhydrase of a plant, such as rice, maize, soybean, tomato, barley, cucumber, alfalfa, bean, pea, pear, almond, or mung bean, or a carbonic anhydrase of another plant, such as tobacco, cotton, aspen, or Arabidopsis. Consistent with the transgenic land plant, the carbonic anhydrase can be, for example, a carbonic anhydrase of a eukaryotic algae.

Accordingly, in some examples the transgenic land plant is modified to express carbonic anhydrase targeted to mitochondria. Also in some examples, the carbonic anhydrase is a carbonic anhydrase of rice, maize, soybean, canola, camelina, tomato, barley, cucumber, alfalfa, bean, pea, pear, almond, or mung bean that is targeted to mitochondria. Also in some examples, the carbonic anhydrase is a carbonic anhydrase of tobacco, cotton, aspen, or Arabidopsis that is targeted to mitochondria. Also in some examples, the carbonic anhydrase is a carbonic anhydrase of a eukaryotic algae that is targeted to mitochondria.

Another aspect of the present invention to further increase seed yield comprises introducing one or more genes selected from a polynucleotide encoding a ferredoxin polypeptide from a bacterial and/or an archaeal species and/or a gene encoding a biotin ligase polypeptide, wherein said heterologous polynucleotide is from a bacterial and/or an archaeal species. This is described in U.S. Provisional Patent Application No. 62/194,550 to North Carolina State University.

### EXAMPLES

### Example 1

### CCP1 Orthologs in Algae

### Eukaryotic Algae Homology Searches

Various BLAST searches (e.g. tblastn; http://blast.ncbi.nlm.nih.gov/Blast.cgi) were conducted using a translated nucleotide database, a whole genome shotgun (also termed WGS) database, and a transcriptome assembly (also termed TSA) database to find sequences highly similar to the CCP1 protein from Chlamydomonas reinhardtii in algae species (TABLE 1). The percent homology of the translated algae sequence was compared to the CCP1 amino acid sequence using the alignment feature of VectorNTl software. Sequences containing between 82% and 18% homology were obtained, as shown in TABLE 1. Several publically available web programs were used to predict putative transmembrane regions to further characterize the algae sequences including Motif Finder (http://www.genome.jp/tools/motif/; TABLE 1), ProSite (http://prosite.expasy.org/; TABLE 1), and Phobius (http://phobius.sbc.su.se/; FIG. 1, FIG. 2, FIG. 3, FIG. 4, FIG. 5, FIG. 6, FIG. 7, and FIG. 8). The Motif Finder program predicts both CCP1 and the algae orthologs as Mito_carr (PF00153) or mitochondrial carrier proteins (TABLE 1). This class of proteins carries molecules across the membrane of mitochondria (http://pfam.xfam.org/family/PF00153). The ProSite program predicted both CCP1 and the algae orthologs as SOLCAR (PS50920) or solute carrier proteins (TABLE 1). This class of proteins are defined as substrate carrier proteins involved in energy transfer in the inner mitochondrial membrane (http://prosite.expasy.org/cgi-bin/prosite/nicedoc.pl?PS50920). Mapping of predicted transmembrane regions of CCP1 and comparing the results to the orthologs with the highest homology was used to further characterize the proteins (FIGS. 1-8). Based on the combined analyses of TABLE 1 and FIG. 1, FIG. 2, FIG. 3, FIG. 4, FIG. 5, FIG. 6, FIG. 7, and FIG. 8, the Gonium pectorale protein (annotated protein ID KXZ50472.1) is the most similar to the Chlamydomonas reinhardtii protein encoded by gene XM_0016921451.

### Multiple Sequence Alignment

A multiple sequence alignment of CCP1 of Chlamydomonas reinhardtii and eleven orthologs of CCP1 of eukaryotic algae as identified based on homology searches was prepared using a Multiple Sequence Alignment tool (EMBL-EBI; http://www.ebi.ac.uk/Tools/msa/clustalo/). Specifically, CCP1 of Chlamydomonas reinhardtii of SEQ ID NO: 1, a mitochondrial transporter protein of a Chlorella sorokiniana of SEQ ID NO: 2, mitochondrial transporter proteins of a Chlorella variabilis of SEQ ID NO: 3, SEQ ID NO: 4, SEQ ID NO: 5, and SEQ ID NO: 6, mitochondrial transporter proteins of a Chondrus crispus of SEQ ID NO: 7, SEQ ID NO: 8, and SEQ ID NO: 9, mitochondrial transporter proteins of Gonium pectorale of SEQ ID NO: 19, or SEQ ID NO: 20, and a mitochondrial transporter protein of Volvox carteri of SEQ ID NO: 21 were aligned by CLUSTAL, using default parameters (dealign input sequences [no]; MBED-like clustering guide-tree [yes]; MBED-like clustering iteration [yes]; number of combined iterations [default(0)]; max guide tree iterations [default −1)]; max HMM iterations [default(−1)]; and order [aligned]). Results are shown in FIG. 9A-C.

With reference to FIG. 9A-C and TABLE 1, structural features and characteristics shared among the various orthologs of CCP1 include (i) (a) a proline residue at position 268, (b) an aspartate residue or glutamine residue at position 270, (c) a lysine residue or arginine residue at position 273, and (d) a serine residue or threonine residue at position 274, with numbering of positions relative to CCP1 of Chlamydomonas reinhardtii of SEQ ID NO: 1, and (ii) an overall identity of at least 15%. Structural features and characteristics shared among the various orthologs of CCP1 also include (i) (a) a glycine residue at position 301, (b) a glycine residue at position 308, and (c) an arginine residue at position 315, with numbering of positions relative to CCP1 of Chlamydomonas reinhardtii of SEQ ID NO: 1, and (ii) an overall identity of at least 15%. With reference to FIG. 1, FIG. 2, FIG. 3, FIG. 4, FIG. 5, FIG. 6, FIG. 7, FIG. 8, and TABLE 1, structural features and characteristics shared among the various orthologs of CCP1 also include a potential transmembrane region between about positions 245 to 265, with numbering of positions relative to CCP1 of Chlamydomonas reinhardtii of SEQ ID NO: 1. Noted amino acid residues, i.e. proline residue at position 268, aspartate residue or glutamine residue at position 270, lysine residue or arginine residue at position 273, and serine residue or threonine residue at position 274, with numbering of positions relative to CCP1 of Chlamydomonas reinhardtii of SEQ ID NO: 1, occur at or after the C-terminal portion of this potential transmembrane region of each of CCP1 and the orthologs. Conservation of the noted amino acid residues, in combination with an overall identity of at least 15%, suggests a structure/function relationship shared among CCP1 and the orthologs.

TABLE 1
Proteins with homology to Chlamydomonas reinhardtii CCP1 in algae

| | Nucleotide | | | | | | | |
|---|---|---|---|---|---|---|---|---|
| | Accession | | Homology to CCP1 | | | | | |
| | (and SEQ ID NO of | Number | Consensus | Identity | | | | |
| | corresponding | of amino | positions | Positions | Program | | | |
| Organism | protein) | acids | (%) | (%) | Motif Finder^{b} | | ProSite^{c} | |
| Chlamydomonas | XM_001692145.1 | 358 | 100 | 100 | Mitochondrial | 3 predicted | Solute | 3 predicted motifs |
| reinhardtii | (SEQ ID NO: 1) | | | | carrier protein | motifs spanning | carrier | spanning amino |
| | | | | | | amino acids 28-119; | protein^{d} | acids 22-118; |
| | | | | | | 129-235; & | | 131-231; & 246-333 |
| | | | | | | 245-334 | | |
| Gonium pectorale | KXZ50472.1 | 356 | 93.3 | 82 | Mitochondrial | 3 predicted | Solute | 3 predicted motifs |
| | (SEQ ID NO: 19) | | | | carrier protein | motifs spanning | carrier | spanning amino |
| | | | | | | amino acids 27-119; | protein^{d} | acids 22-118; |
| | | | | | | 129-234; & | | 128-230; & 245-332 |
| | | | | | | 244-333 | | |
| Gonium pectorale | KXZ50486.1 | 354 | 90.8 | 81.9 | Mitochondrial | 3 predicted | Solute | 3 predicted motifs |
| | (SEQ ID NO: 20) | | | | carrier protein | motifs spanning | carrier | spanning amino |
| | | | | | | amino acids 27-119; | protein^{d} | acids 22-118; |
| | | | | | | 129-234; & | | 128-230; & 245-332 |
| | | | | | | 244-333 | | |
| Volvox carteri | XM_002951197.1 | 339 | 88.8 | 80 | Mitochondrial | 3 predicted | Solute | 3 predicted motifs |
| | (SEQ ID NO: 21) | | | | carrier protein | motifs spanning | carrier | spanning amino |
| | | | | | | amino acids 21-112; | protein^{d} | acids 15-111; |
| | | | | | | 122-215; & | | 121-212; & 227-314 |
| | | | | | | 227-315 | | |
| Chlorella | GAPD01006726.1 | 354^{a} | 72.8 | 59.9 | Mitochondrial | 3 predicted | Solute | 3 predicted motifs |
| sorokiniana | (SEQ ID NO: 2) | | | | carrier protein | motifs spanning | carrier | spanning amino |
| | | | | | | amino acids 25-117; | protein^{d} | acids 20-116; |
| | | | | | | 128-228; & | | 131-227; & 238-325 |
| | | | | | | 243-329 | | |
| Chlorella | XM_005846489.1 | 303 | 42.5 | 25.8 | Mitochondrial | 3 predicted | Solute | 3 predicted motifs |
| variabilis | (SEQ ID NO: 6) | | | | carrier protein | motifs spanning | carrier | spanning amino |
| | | | | | | amino acids 4-88; | protein^{d} | acids 3-86; 96-200; |
| | | | | | | 97-199; & | | & 212-300 |
| | | | | | | 212-301 | | |
| Chlorella | XM_005852157.1 | 323 | 40.3 | 25.2 | Mitochondrial | 3 predicted | Solute | 3 predicted motifs |
| variabilis | (SEQ ID NO: 4) | | | | carrier protein | motifs spanning | carrier | spanning amino |
| | | | | | | amino acids 26-115; | protein^{d} | acids 25-112; |
| | | | | | | 125-221; & | | 124-218; & 230-319 |
| | | | | | | 229-322 | | |
| Chlorella | XM_005843001.1 | 323 | 39.3 | 24.7 | Mitochondrial | 3 predicted | Solute | 3 predicted motifs |
| variabilis | (SEQ ID NO: 5) | | | | carrier protein | motifs spanning | carrier | spanning amino |
| | | | | | | amino acids 9-90; | protein^{d} | acids 8-92; 101-189; |
| | | | | | | 108-187; & | | & 221-308 |
| | | | | | | 225-307 | | |
| Chondrus crispus | XM_005712871.1 | 328 | 34.7 | 20.3 | Mitochondrial | 3 predicted | Solute | 3 predicted motifs |
| | (SEQ ID NO: 7) | | | | carrier protein | motifs spanning | carrier | spanning amino |
| | | | | | | amino acids 40-127; | protein^{d} | acids 39-128; 135-227; |
| | | | | | | 137-230; & | | & 238-325 |
| | | | | | | 239-326 | | |
| Chlorella | XM_005851446.1 | 306 | 35.8 | 21.7 | Mitochondrial | 3 predicted | Solute | 3 predicted motifs |
| variabilis | (SEQ ID NO: 3) | | | | carrier protein | motifs spanning | carrier | spanning amino |
| | | | | | | amino acids 11-101; | protein^{d} | acids 11-100; |
| | | | | | | 112-206; & | | 112-203; & 212-298 |
| | | | | | | 213-299 | | |
| Chondrus crispus | XM_005715654.1 | 233 | 35.2 | 22.9 | Mitochondrial | 3 predicted | Solute | 3 predicted motifs |
| | (SEQ ID NO: 8) | | | | carrier protein | motifs spanning | carrier | spanning amino |
| | | | | | | amino acids 3-40; | protein^{d} | acids 1-37; 47-131; |
| | | | | | | 47-132; & | | & 142-229 |
| | | | | | | 141-231 | | |
| Chondrus crispus | XM_005713259.1 | 194 | 29.9 | 18.4 | Mitochondrial | 2 predicted | Solute | 2 predicted motifs |
| | (SEQ ID NO: 9) | | | | carrier protein | motifs spanning | carrier | spanning amino |
| | | | | | | amino acids 7-93 | protein^{d} | acids 8-92 & 103-190 |
| | | | | | | & 102-191 | | |

^{a}sequence from first methionine of deposited transcribed mRNA sequence to first stop codon
^{b}http://www.genome.jp/tools/motif/
^{c}http://prosite.expasy.org/
^{d}predicted as one of several substrate carrier proteins involved in energy transfer in the inner mitochondrial membrane (http://prosite.expasy.org/cgi-bin/prosite/nicedoc.pl?PS50920)

### Example 2

### Preparation of Genetic Constructs Encoding Algae Orthologs of CCP1

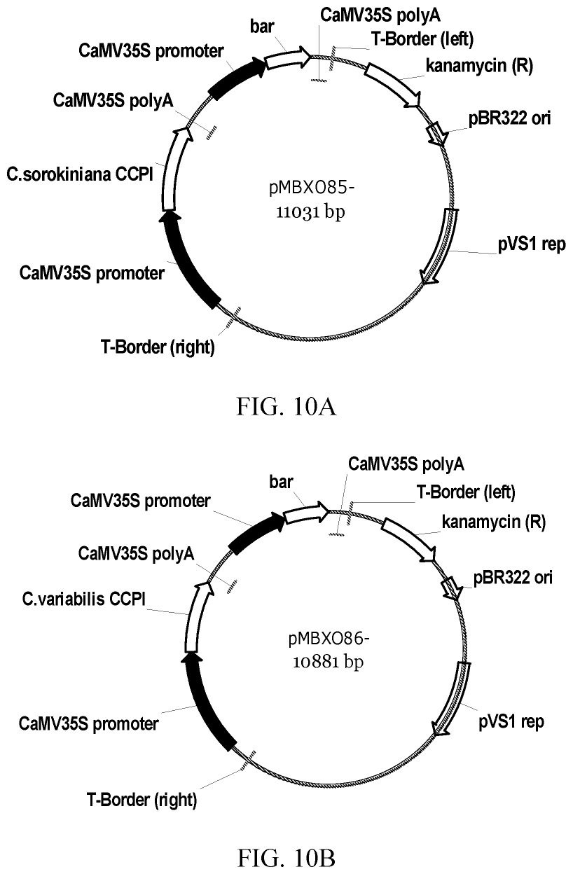

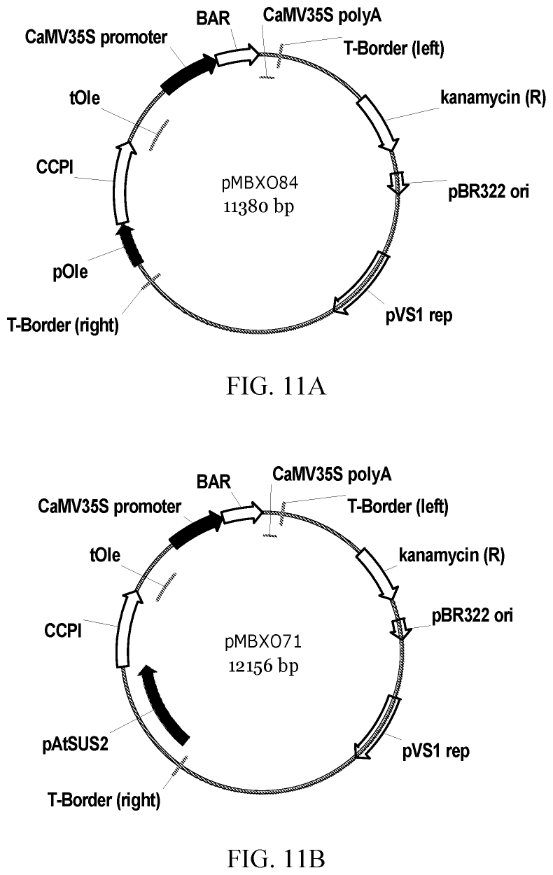

Genetic constructs pMBXO85 (SEQ ID NO: 10) and pMBXO86 (SEQ ID NO: 11) contain orthologs of CCP1 from algae and are derivatives of pCAMBIA binary vectors (Centre for Application of Molecular Biology to International Agriculture, Canberra, Australia). These plasmids were constructed using cloning techniques that are standard to those skilled in the art. The source of orthologs of the CCP1 gene encoded by these genetic constructs, as well as the promoter driving the expression of the CCP1 ortholog, are listed in TABLE 2. Both pMBXO85 and pMBXO86 have a constitutive expression cassette for the bar gene, that imparts transgenic plants resistance to the herbicide bialophos allowing for their selection. Maps of pMBXO85 and pMBXO86 illustrating the plant expression elements for directing the expression of the CCP1 orthologs in plants are shown in FIG. 10A and FIG. 10B, respectively.

TABLE 2
Summary of constructs for transformation into Camelina

| Construct name | Promoter | Source of CCP1 ortholog gene |
|---|---|---|
| pMBXO85 | 35sCAMV | Chlorella sorokiniana |
| (SEQ ID NO: 10) | (constitutive) | |
| pMBXO86 | 35sCAMV | Chlorella variabilis |
| (SEQ ID NO: 11) | (constitutive) | |

### Example 3

### Transformation of Genetic Constructs Encoding Algae Orthologs of CCP1 Under the Expression Control of a Plant Constitutive Promoter into Camelina sativa

In preparation for plant transformation experiments, seeds of Camelina sativa germplasm 10CS0043 (abbreviated WT43, obtained from Agriculture and Agri-Food Canada) were sown directly into 4 inch pots filled with soil in the greenhouse. Growth conditions were maintained at 24° C. during the day and 18° C. during the night. Plants were grown until flowering. Plants with a number of unopened flower buds were used in ‘floral dip’ transformations.

Agrobacterium strain GV3101 (pMP90) was transformed with either pMBXO85 or pMBXO86 using electroporation. A single colony of GV3101 (pMP90) containing the construct of interest was obtained from a freshly streaked plate and was inoculated into 5 mL LB medium. After overnight growth at 28° C., 2 mL of culture was transferred to a 500-mL flask containing 300 mL of LB and incubated overnight at 28° C. Cells were pelleted by centrifugation (6,000 rpm, 20 min), and diluted to an OD600 of ˜0.8 with infiltration medium containing 5% sucrose and 0.05% (v/v) Silwet-L77 (Lehle Seeds, Round Rock, Tex., USA). Camelina plants were transformed by “floral dip” using the pMBXO85 and pMBXO86 transformation constructs as follows. Pots containing plants at the flowering stage were placed inside a 460 mm height vacuum desiccator (Bel-Art, Pequannock, N.J., USA). Inflorescences were immersed into the Agrobacterium inoculum contained in a 500-ml beaker. A vacuum (85 kPa) was applied and held for 5 min. Plants were removed from the desiccator and were covered with plastic bags in the dark for 24 h at room temperature. Plants were removed from the bags and returned to normal growth conditions within the greenhouse for seed formation (T1 generation of seed).

T1 seeds were planted in soil and transgenic plants were selected by spraying a solution of 400 mg/L of the herbicide Liberty (active ingredient 15% glufosinate-ammonium). This allows identification of transgenic plants containing the bar gene on the T-DNA in the plasmid vectors pMBXO85 and pMBXO86 (FIG. 10). Transgenic plant lines were further confirmed using PCR with primers specific to the algae ortholog gene of interest. PCR positive lines were grown in a greenhouse to produce the next generation of seed (T2 seed). Seeds were isolated from each plant and were dried in an oven with mechanical convection set at 22° C. for two days. The weight of the entire harvested seed obtained from individual plants was measured and recorded. Multiple T1 plants from pMBXO85 and pMBXO86 plants produced more T2 seed than wild-type controls. The best line from the pMBXO85 transformation produced 54% more seed than wild-type controls whereas the best pMBXO86 line produced 30% more seed than controls.

TABLE 3
T2 seed yield in lines of Camelina
transformed with pMBXO85 and pMBXO86.

| Genetic | | Seed | % of wild- |
|---|---|---|---|
| Construct | Line | Yield (g) | type control |
| None | Wild-type ¹ | 4.39 ± 1.42 | 100 |
| pMBXO85 | 16-0889 | 6.76 | 154 |
| | 16-0886 | 6.15 | 140 |
| | 16-0894 | 6.1 | 139 |
| | 16-0895 | 5.82 | 133 |
| | 16-0896 | 5.48 | 125 |
| | 16-0888 | 5.24 | 119 |
| | 16-0891 | 5.24 | 119 |
| | 16-0897 | 5.1 | 116 |
| | 16-0903 | 4.86 | 111 |
| | 16-0893 | 4.81 | 110 |
| | 16-0932 | 4.76 | 109 |
| | 16-0892 | 4.61 | 105 |
| | 16-0920 | 4.55 | 104 |
| pMBXO86 | 16-0839 | 5.68 | 130 |
| | 16-0853 | 4.7 | 107 |

¹ Wild-type control seed yield values are an average of 25 plants.

### Example 4

### Preparation of Genetic Constructs pMBXO84, pMBXO71, and pMBXO107 for Seed Specific Expression of Chlamydomonas reinhardtii CCP1 Gene in Camelina sativa

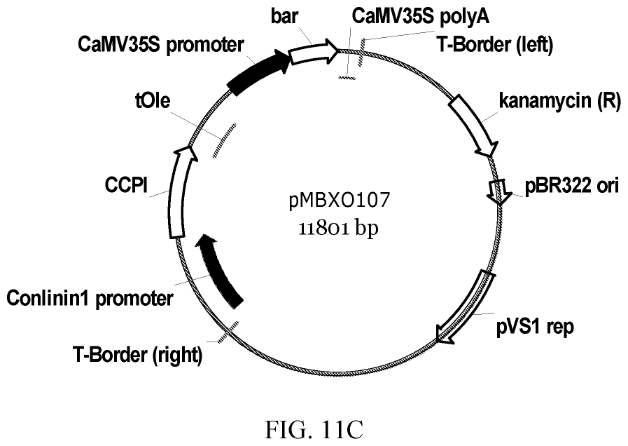

Genetic constructs pMBXO84, pMBXO71, and pMBXO107 contain the CCP1 gene from C. reinhardtii expressed from seed specific promoters (TABLE 4). The plasmids are derivatives of pCAMBIA binary vectors (Centre for Application of Molecular Biology to International Agriculture, Canberra, Australia). These plasmids were constructed using cloning techniques that are standard to those skilled in the art. The plasmids pMBXO84, pMBXO71, and pMBXO107 have a constitutive expression cassette for the bar gene, that imparts transgenic plants resistance to the herbicide bialophos allowing for their selection. Plasmid maps of pMBXO84, pMBXO71, and pMBXO107 illustrating the plant expression elements for directing the seed specific expression of the gene encoding the C. reinhardtii CCP1 in plants are shown in FIG. 11.

TABLE 4
Summary of transformation constructs for seed
specific expression of CCP1 in Camelina

| Construct name | Promoter | Source of gene encoding CCP1 |
|---|---|---|
| pMBXO84 | Soya bean oleosin 1 | Chlamydomonas reinhardtii |
| | isoform A gene | |
| | (seed-specific) | |
| pMBXO71 | A. thaliana | Chlamydomonas reinhardtii |
| | sucrose synthase | |
| | (seed-specific) | |
| pMBXO107 | Flax conlinin promoter | Chlamydomonas reinhardtii |
| | (seed-specific) | |

Camelina sativa germplasm WT43 was transformed with genetic constructs pMBXO84, pMBXO71, and pMBXO107 as described above and the first generation (T1) of seed was obtained. Seeds were sowed in soil and a solution of the herbicide bialophos was sprayed on the plants, as described above, to identify transgenics. All putative transgenics were confirmed by PCR. Transgenic plants were grown to produce T2 seed and the total seed was harvested from the plant, dried in an oven with mechanical convection set at 22° C. for two days.

For pMBXO71, the weight of the entire harvested seed obtained from individual plants was measured and recorded and is shown in TABLE 5. Up to a ˜60% increase in seed weight compared to wild-type controls was observed in individual plants.

TABLE 5
T2 seed yield in lines of Camelina transformed with pMBXO71.

| Genetic | | Seed | % of wild- |
|---|---|---|---|
| Construct | Line | Yield (g) | type control |
| None | Wild-type ¹ | 4.39 ± 1.42 | 100 |
| pMBXO71 | 16-0788 | 7.09 | 162 |
| | 16-0787 | 6.61 | 151 |
| | 16-0800 | 6.03 | 138 |
| | 16-0789 | 5.90 | 135 |
| | 16-0794 | 5.55 | 127 |
| | 16-0797 | 5.3 | 121 |
| | 16-0796 | 5.04 | 115 |
| | 16-0808 | 5.01 | 114 |
| | 16-0786 | 4.98 | 114 |
| | 16-0810 | 4.98 | 114 |
| | 16-0795 | 4.91 | 112 |
| | 16-0791 | 4.86 | 105 |
| | 16-0809 | 4.6 | 105 |
| | 16-0792 | 4.53 | 103 |
| | 16-0799 | 4.49 | 102 |

¹ Wild-type control seed yield values are an average of 25 plants. T2 seed yield is data from one individual plant.

For construct pMBXO84, ˜290 T1 lines were obtained from floral dip transformation. T1 lines with 1 and 2 copy numbers, and with seed yields comparable or superior to the wild-type growing in the vicinity of the transgenic line, were advanced to T3 and T4 generations to isolate lines with improved seed yield. To obtain T4 seed, T2 seeds were sowed in soil and allowed to produce T3 seed which was then harvested. Multiple T3 seed for each line (9-10 seeds) were planted in soil and allowed to produce T4 seed. The T4 seed was harvested separately for the replicates of each line and seed yield, oil content, and 100 seed weight were measured.

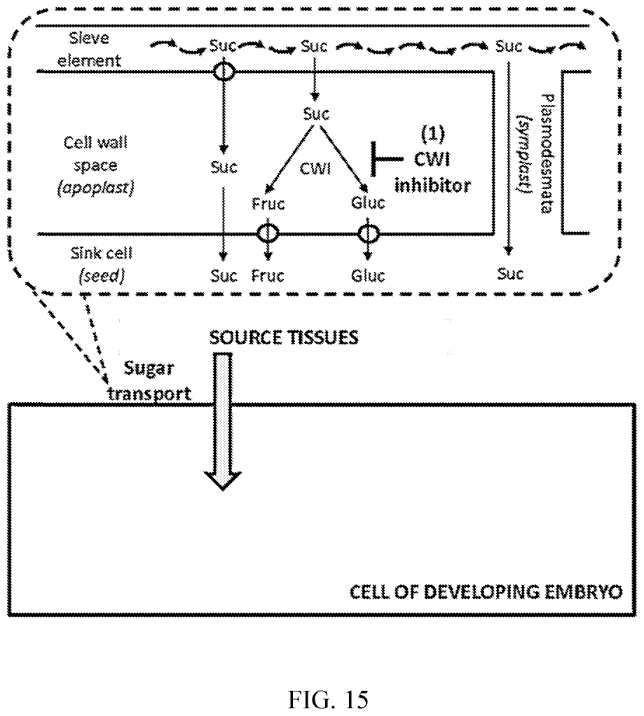

For the pMBXO84 construct, an up to 24% increase in seed yield was observed for the best line compared to wild-type controls (TABLE 6). Oil content remained essentially the same as wild-type controls for lines transformed with pMBXO84 (TABLE 7) but the weight of 100 seeds of the transgenic lines increased by up to 14% (TABLE 8). The increased yield per plant and the increased 100 seed weight for seed specific expression of C. reinhardtii CCP1 is an unexpected result. Previous experiments where the C. reinhardtii CCP1 gene was expressed in Camelina from a constitutive promoter produced a higher yield of seeds compared to wild-type controls (FIG. 15 in WO 2015/103074,) but produced smaller seeds with a reduced 100 seed weight of almost 20% (FIG. 16 in WO 2015/103074).

TABLE 6
T4 seed yield per plant from lines of
Camelina transformed with pMBXO84.

| Genetic | | Copy | Seed Yield of | % of wild- |
|---|---|---|---|---|
| Construct | Event | number | Plant¹ (g) | type control |
| None | Wild-type | Not | 7.75 +/− 2.58 | 100 |
| | | applicable | | |
| pMBXO84 | ND04 | 1 | 9.64 +/− 1.79 | 124 |
| | ND78 | 1 | 9.44 +/− 3.33 | 122 |
| | ND16 | 2 | 8.88 +/− 3.34 | 115 |
| | ND18 | 1 | 8.72 +/− 2.07 | 112 |
| | ND79 | 2 | 8.53 +/− 2.29 | 110 |
| | ND48 | 2 | 8.28 +/− 1.13 | 107 |

¹Seed yield values are an average of 10 plants for all lines with the exception of ND18 and ND79 where only 9 plants were available.

TABLE 7
Oil content of T4 seed from lines of
Camelina transformed with pMBXO84.

| Genetic | | Copy | | % of wild- |
|---|---|---|---|---|
| Construct | Event | number | Oil content | type control |
| None | Wild-type | Not | 31.6 +/− 1.3 | 100 |
| | | applicable | | |
| pMBXO84 | ND04 | 1 | 31.6 +/− 1.5 | 100 |
| | ND78 | 1 | 32.2 +/− 1.7 | 102 |
| | ND16 | 2 | 31.6 +/− 1.2 | 100 |
| | ND18 | 1 | 31.2 +/− 1.5 | 99 |
| | ND79 | 2 | 30.9 +/− 1/3 | 98 |
| | ND48 | 2 | 31.7 +/− 1/3 | 100 |

¹ Seed yield values are an average of 10 plants for all lines with the exception of ND18 and ND79 where only 9 plants were available.

TABLE 8
100 seed weight of T4 seed from lines of
Camelina transformed with pMBXO84.

| Genetic | | Copy | 100 seed | % wild- |
|---|---|---|---|---|
| Construct | Event | number | weight¹ (g) | type control |
| None | Wild-type | Not | 0.117 +/− 0.008 | 100 |
| | | applicable | | |
| pMBXO84 | ND04 | 1 | 0.127 +/− 0.014 | 108 |
| | ND78 | 1 | 0.129 +/− 0.012 | 110 |
| | ND16 | 2 | 0.133 +/− 0.012 | 114 |
| | ND18 | 1 | 0.123 +/− 0.012 | 105 |
| | ND79 | 2 | 0.120 +/− 0.011 | 103 |
| | ND48 | 2 | 0.122 +/− 0.006 | 104 |

¹Seed yield values are an average of 10 plants for all lines with the exception of ND18 and ND79 where only 9 plants were available.

Plasmid pMBXO107 can similarly be transformed into Camelina and plants screened for increased seed yield using the procedures above.

As will be appreciated, these genetic constructs and others may be used for seed-specific expression of the CCP1 gene from C. reinhardtii in other land plants. Moreover, similar genetic constructs can be made for seed specific expression of a mitochondrial transporter protein of a Chlorella sorokiniana of SEQ ID NO: 2, mitochondrial transporter proteins of a Chlorella variabilis of SEQ ID NO: 3, SEQ ID NO: 4, SEQ ID NO: 5, and SEQ ID NO: 6, mitochondrial transporter proteins of a Chondrus crispus of SEQ ID NO: 7, SEQ ID NO: 8, and SEQ ID NO: 9, mitochondrial transporter proteins of a Gonium pectorale of SEQ ID NO: 19 or SEQ ID NO: 20, and a mitochondrial transporter protein of a Volvox carteri of SEQ ID NO: 21, in Camelina sativa and other land plants.

### Example 5

### Seed Specific Expression of C. reinhardtii CCP1 in Canola

In preparation for plant transformation experiments, seeds of Brassica napus cv DH12075 (obtained from Agriculture and Agri-Food Canada) were surface sterilized with sufficient 95% ethanol for 15 seconds, followed by 15 minutes incubation with occasional agitation in full strength Javex (or other commercial bleach, 7.4% sodium hypochlorite) and a drop of wetting agent such as Tween 20. The Javex solution was decanted and 0.025% mercuric chloride with a drop of Tween 20 was added and the seeds were sterilized for another 10 minutes. The seeds were then rinsed three times with sterile distilled water. The sterilized seeds were plated on half strength hormone-free Murashige and Skoog (MS) media (Murashige T, Skoog F (1962). Physiol Plant 15:473-498) with 1% sucrose in 15×60 mm petri dishes that were then placed, with the lid removed, into a larger sterile vessel (Majenta GA7 jars). The cultures were kept at 25° C., with 16 h light/8 h dark, under approx. 70-80 μE of light intensity in a tissue culture cabinet. 4-5 days old seedlings were used to excise fully unfolded cotyledons along with a small segment of the hypocotyl. Excisions were made so as to ensure that no part of the apical meristem was included.

The Agrobacterium strain GV3101 carrying the pMBXO84 (FIG. 11A) seed specific expression plasmid was grown overnight in 5 ml of LB media with 50 mg/L kanamycin, gentamycin, and rifampicin. The culture was centrifuged at 2000 g for 10 min., the supernatant was discarded and the pellet was suspended in 5 ml of inoculation medium (Murashige and Skoog with B5 vitamins [MS/B5; Gamborg O L, Miller R A, Ojima K. Exp Cell Res 50:151-158], 3% sucrose, 0.5 mg/L benzyl aminopurine (BA), pH 5.8). Cotyledons were collected in Petri dishes with ˜1 ml of sterile water to keep them from wilting. The water was removed prior to inoculation and explants were inoculated in mixture of 1 part Agrobacterium suspension and 9 parts inoculation medium in a final volume sufficient to bathe the explants. After explants were well exposed to the Agrobacterium solution and inoculated, a pipet was used to remove any extra liquid from the petri dishes.

The Petri plates containing the explants incubated in the inoculation media were sealed and kept in the dark in a tissue culture cabinet set at 25 ° C. After 2 days the cultures were transferred to 4 ° C. and incubated in the dark for 3 days. The cotyledons, in batches of 10, were then transferred to selection medium consisting of Murashige Minimal Organics (Sigma), 3% sucrose, 4.5 mg/L BA, 500 mg/L MES, 27.8 mg/L Iron (II) sulfate heptahydrate, pH 5.8, 0.7% Phytagel with 300 mg/L timentin, and 2 mg/L L-phosphinothricin (L-PPT) added after autoclaving. The cultures were kept in a tissue culture cabinet set at 25° C., 16 h/8 h, with a light intensity of about 125 μmol m⁻² s⁻¹. The cotyledons were transferred to fresh selection every 3 weeks until shoots were obtained. The shoots were excised and transferred to shoot elongation media containing MS/B5 media, 2% sucrose, 0.5 mg/L BA, 0.03 mg/L gibberellic acid (GA₃), 500 mg/L 4-morpholineethanesulfonic acid (MES), 150 mg/L phloroglucinol, pH 5.8, 0.9% Phytagar and 300 mg/L timentin and 3 mg/L L-phosphinothricin added after autoclaving. After 3-4 weeks any callus that was formed at the base of shoots with normal morphology was cut off and shoots were transferred to rooting media containing half strength MS/B5 media with 1% sucrose and 0.5 mg/L indole butyric acid, 500 mg/L MES, pH 5.8, 0.8% agar, with 1.5 mg/L L-PPT and 300 mg/L timentin added after autoclaving. The plantlets with healthy shoots were hardened and transferred to 6″ pots in the greenhouse to collect T1 transgenic seeds.

Plasmids pMBXO71 and pMBXO107 can similarly be transformed into canola using the procedures above.

### Example 6

### Screening of Transgenic Plants of Canola Expressing Seed Specific CCP1 and Identification of Plants with Higher Yield

Canola T0 lines transformed with the plasmid vector pMBXO84 were generated and grown to produce T1 seed. The copy number of each line was determined using Southern blotting techniques. The T1 seeds of several independent lines (TABLE 9) were grown in a greenhouse maintained at 24° C. during the day and 18° C. during the night to produce T2 seeds. All T1 plants of pMBXO84 were sprayed with 400 mg/L of the herbicide Liberty to select for transformed plants.

TABLE 9
T1 lines of Camelina transformed with
pMBXO84 advanced to produce T2 seed.

| Genetic Construct | Event | Copy number |
|---|---|---|
| pMBXO84 | OP05 | 1 |
| | OP12 | 1 |
| | OP22 | 1 |
| | OP43 | 1 |
| | OP48 | 1 |
| | OP29 | 2 |
| | OP45 | 2 |
| | OP13 | 3 |
| | OP14 | 4 |

Seed yield from each plant is determined by harvesting all of the mature seeds from a plant and drying them in an oven with mechanical convection set at 22° C. for two days. The weight of the entire harvested seed is recorded.

Canola T0 lines transformed with the plasmid vectors pMBXO71 and pMBXO107 are generated. The Ti seeds of several independent lines are grown in a randomized complete block design in a greenhouse maintained at 24° C. during the day and 18° C. during the night. The T2 generation of seed from each line is harvested. Seed yield from each plant is determined by harvesting all of the mature seeds from a plant and drying them in an oven with mechanical convection set at 22° C. for two days. The weight of the entire harvested seed is recorded. The 100 seed weight is measured to obtain an indication of seed size.

### Example 7

### Seed Specific Expression of C. reinhardtii CCP1 from Plasmid pMBXO75 in Soybean

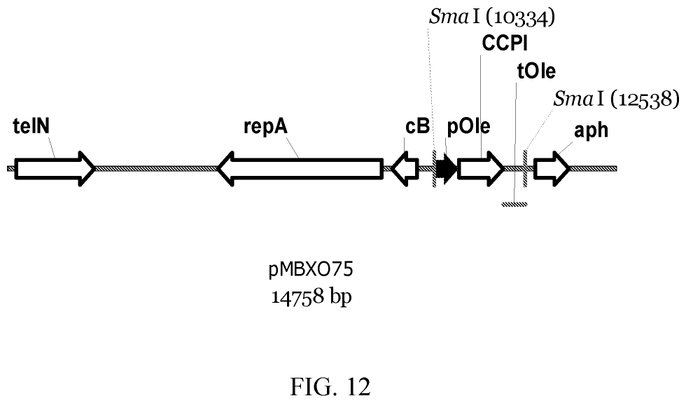

Plasmid pMBXO75 is a derivative of the pJAZZ linear vector (Lucigen, Inc.) and was constructed using cloning techniques standard for those skilled in the art (FIG. 12). The vector contains the C. reinhardtii CCP1 gene, codon optimized for expression in soybean, under the control of a seed-specific promoter from the soya bean oleosin isoform A gene. The cloning was designed to enable the excision of the CCP1 expression cassette, using restriction digestion, from the vector backbone. A 2.2 kb SmaI DNA fragment containing the expression cassette consisting of oleosin promoter, CCP1, and oleosin terminator was excised from the pMBXO75. The purified DNA fragment containing the CCP1 expression cassettes was co-bombarded with DNA encoding an expression cassette for the hygromycin resistance gene via biolistics into embryogenic cultures of soybean Glycine max cultivars X5 and Westag97, to obtain transgenic plants. The transformation, selection, and plant regeneration protocol was adapted from Simmonds (2003) (Simmonds, 2003, Genetic Transformation of Soybean with Biolistics. In: Jackson J F, Linskens H F (eds) Genetic Transformation of Plants. Springer Verlag, Berlin, pp 159-174) and was performed as follows.

Induction and Maintenance of Proliferative Embryogenic Cultures: Immature pods, containing 3-5 mm long embryos, were harvested from host plants grown at 28/24° C. (day/night), 15-h photoperiod at a light intensity of 300-400 μmol m⁻² s⁻¹. Pods were sterilized for 30 s in 70% ethanol followed by 15 min in 1% sodium hypochlorite [with 1-2 drops of Tween 20 (Sigma, Oakville, ON, Canada)] and three rinses in sterile water. The embryonic axis was excised and explants were cultured with the abaxial surface in contact with the induction medium [MS salts, B5 vitamins (Gamborg O L, Miller R A, Ojima K. Exp Cell Res 50:151-158), 3% sucrose, 0.5 mg/L BA, pH 5.8), 1.25-3.5% glucose (concentration varies with genotype), 20 mg/1 2,4-D, pH 5.7]. The explants, maintained at 20° C. at a 20-h photoperiod under cool white fluorescent lights at 35-75 μmol m⁻² s⁻¹, were sub-cultured four times at 2-week intervals. Embryogenic clusters, observed after 3-8 weeks of culture depending on the genotype, are transferred to 125-ml Erlenmeyer flasks containing 30 ml of embryo proliferation medium containing 5 mM asparagine, 1-2.4% sucrose (concentration is genotype dependent), 10 mg/12,4-D, pH 5.0 and cultured as above at 35-60 μmol m⁻² s⁻¹ of light on a rotary shaker at 125 rpm. Embryogenic tissue (30-60 mg) was selected, using an inverted microscope, for subculture every 4-5 weeks.

Transformation: Cultures were bombarded 3 days after subculture. The embryogenic clusters were blotted on sterile Whatman filter paper to remove the liquid medium, placed inside a 10×30-mm Petri dish on a 2×2 cm² tissue holder (PeCap, 1 005 μm pore size, Band SH Thompson and Co. Ltd. Scarborough, ON, Canada) and covered with a second tissue holder that is then gently pressed down to hold the clusters in place. Immediately before the first bombardment, the tissue was air dried in the laminar air flow hood with the Petri dish cover off for no longer than 5 min. The tissue was turned over, dried as before, bombarded on the second side and returned to the culture flask. The bombardment conditions used for the Biolistic PDS-I000/He Particle Delivery System are as follows: 737 mm Hg chamber vacuum pressure, 13 mm distance between rupture disc (Bio-Rad Laboratories Ltd., Mississauga, ON, Canada) and macrocarrier. The first bombardment used 900 psi rupture discs and a microcarrier flight distance of 8.2 cm, and the second bombardment used 1100 psi rupture discs and 11.4 cm microcarrier flight distance. DNA precipitation onto 1.0 μm diameter gold particles was carried out as follows: 2.5 μl of 100 ng/μl of insert DNA of pMBXO75 and 2.5 μl of 100 ng/μl selectable marker DNA (cassette for hygromycin selection) were added to 3 mg gold particles suspended in 50 μl sterile dH₂0 and vortexed for 10 sec; 50 μl of 2.5 M CaCl₂ was added, vortexed for 5 sec, followed by the addition of 20 μl of 0.1 M spermidine which was also vortexed for 5 sec. The gold was then allowed to settle to the bottom of the microfuge tube (5-10 min) and the supernatant fluid was removed. The gold/DNA was resuspended in 200 μl of 100% ethanol, allowed to settle and the supernatant fluid was removed. The ethanol wash was repeated and the supernatant fluid was removed. The sediment was resuspended in 120 μl of 100% ethanol and aliquots of 8 μl were added to each macrocarrier. The gold was resuspended before each aliquot was removed. The macrocarriers were placed under vacuum to ensure complete evaporation of ethanol (about 5 min).

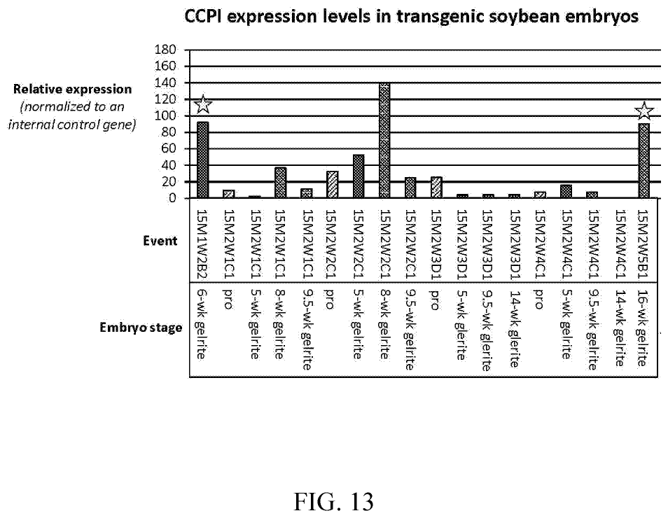

Selection: The bombarded tissue was cultured on embryo proliferation medium described above for 12 days prior to subculture to selection medium (embryo proliferation medium containing 55 mg/l hygromycin added to autoclaved media). The tissue was sub-cultured 5 days later and weekly for the following 9 weeks. Green colonies (putative transgenic events) were transferred to a well containing 1 ml of selection media in a 24-well multi-well plate that was maintained on a flask shaker as above. The media in multi-well dishes was replaced with fresh media every 2 weeks until the colonies were approx. 2-4 mm in diameter with proliferative embryos, at which time they were transferred to 125 ml Erlenmeyer flasks containing 30 ml of selection medium. A portion of the proembryos from transgenic events was harvested to examine gene expression by RT-PCR and transcripts from expression of the CCP1 gene were observed (FIG. 13).

Plant regeneration: Maturation of embryos was carried out, without selection, at conditions described for embryo induction. Embryogenic clusters were cultured on Petri dishes containing maturation medium (MS salts, B5 vitamins, 6% maltose, 0.2% gelrite gellan gum (Sigma), 750 mg/l MgCl₂, pH 5.7) with 0.5% activated charcoal for 5-7 days and without activated charcoal for the following 3 weeks. Embryos (10-15 per event) with apical meristems were selected under a dissection microscope and cultured on a similar medium containing 0.6% phytagar (Gibco, Burlington, ON, Canada) as the solidifying agent, without the additional MgCl₂, for another 2-3 weeks or until the embryos become pale yellow in color. A portion of the embryos from transgenic events after varying times on gelrite were harvested to examine gene expression by RT-PCR and transcripts from expression of the CCP1 gene were observed (FIG. 13).

Mature embryos were desiccated by transferring embryos from each event to empty Petri dish bottoms that are placed inside Magenta boxes (Sigma) containing several layers of sterile Whatman filter paper flooded with sterile water, for 100% relative humidity. The Magenta boxes were covered and maintained in darkness at 20° C. for 5-7 days. The embryos were germinated on solid B5 medium containing 2% sucrose, 0.2% gelrite and 0.075% MgCl₂ in Petri plates, in a chamber at 20° C., 20-h photoperiod under cool white fluorescent lights at 35-75 μmol m⁻² s⁻¹. Germinated embryos with unifoliate or trifoliate leaves were planted in artificial soil (Sunshine Mix No. 3, SunGro Horticulture Inc., Bellevue, Wash., USA), and covered with a transparent plastic lid to maintain high humidity. The flats were placed in a controlled growth cabinet at 26/24° C. (day/night), 18 h photoperiod at a light intensity of 150 μmol m⁻² s⁻¹. At the 2-3 trifoliate stage (2-3 weeks), the plantlets with strong roots were transplanted to pots containing a 3:1:1:1 mix of ASB Original Grower Mix (a peat-based mix from Greenworld, ON, Canada):soil: sand: perlite and grown at 18-h photoperiod at a light intensity of 300-400 μmolm⁻² s⁻¹.

T1 seeds were harvested and planted in soil and grown in a controlled growth cabinet at 26/24° C. (day/night), 18 h photoperiod at a light intensity of 300-400 μmol m⁻² s⁻¹. Plants were grown to maturity and T2 seed was harvested. The number of branches, pods, and seeds was measured for each plant (TABLE 10, TABLE 11, and TABLE 12). The seed yield in grams per plant, as well as the average individual weight per seed was also determined (TABLE 13 and TABLE 14).

TABLE 10
Distribution of pods on transgenic soybean plants transformed
with a seed specific expression cassette for CCP1 from
pMBXO75 compared to wild-type controls

| Line | # of lateral branches | % to wild-type control* |
|---|---|---|
| A6 | 9 | 138 |
| A8 | 9 | 138 |
| A11 | 8 | 123 |
| A12 | 10 | 154 |
| B11 | 8 | 123 |
| B12 | 9 | 138 |
| D12 | 6 | 92 |
| G2 | 9 | 138 |
| H3 | 9 | 138 |
| WT | 6.50 ± 1.05 | 100 |

*% of control is calculated from the average of 6 wild-type control plants (Westag cultivar).
WT, wild-type

TABLE 11
Distribution of pods on transgenic soybean plants transformed
with a seed specific expression cassette for CCP1 from pMBXO75
compared to wild-type controls

| | Lateral branches | | Main Stem | | Total Plant | |
|---|---|---|---|---|---|---|
| | | % of | | % of | | % of |
| Line | # pods | control* | # pods | control* | # pods | control* |
| A6 | 68 | 233 | 38 | 145 | 106 | 192 |
| A8 | 95 | 326 | 25 | 96 | 120 | 217 |
| A11 | 65 | 223 | 37 | 141 | 102 | 184 |
| A12 | 66 | 226 | 36 | 138 | 102 | 184 |
| B11 | 89 | 305 | 49 | 187 | 138 | 249 |
| B12 | 67 | 230 | 25 | 96 | 92 | 166 |
| D12 | 28 | 96 | 24 | 92 | 52 | 94 |
| G2 | 45 | 154 | 30 | 115 | 75 | 136 |
| H3 | 59 | 202 | 38 | 145 | 97 | 175 |
| WT | 29.17 ± 7.44 | 100 | 26.17 ± | 100 | 55.33 ± 6.41 | 100 |
| | | | 2.14 | | | |

*% of control is calculated from the average of 6 wild-type control plants (Westag cultivar).
WT, wild-type

TABLE 12
Number of seeds on lateral branches and main stem of transgenic
soybean plants transformed with a seed specific expression
cassette for CCP1 from pMBXO75 compared to wild-type
controls

| | Lateral branches | | Main Stem | | Total Plant | |
|---|---|---|---|---|---|---|
| | | % of | | % of | | % of |
| Line | # seeds | control* | # seeds | control* | #seeds | control* |
| A6 | 87 | 135 | 54 | 83 | 141 | 109 |
| A8 | 115 | 179 | 28 | 43 | 143 | 111 |
| A11 | 85 | 132 | 51 | 79 | 136 | 105 |
| A12 | 92 | 143 | 55 | 85 | 147 | 114 |
| B11 | 100 | 155 | 56 | 86 | 156 | 121 |
| B12 | 73 | 113 | 32 | 49 | 105 | 81 |
| D12 | 48 | 75 | 40 | 62 | 88 | 68 |
| G2 | 92 | 143 | 62 | 96 | 154 | 119 |
| H3 | 100 | 155 | 67 | 103 | 167 | 129 |
| WT | 64.33 ± | 100 | 64.83 ± 4.54 | 100 | 129.17 ± | 100 |
| | 18.22 | | | | 18.57 | |

*% of control is calculated from the average of 6 wild-type control plants (Westag cultivar).
WT, wild-type

TABLE 13
The yield of seed (grams) obtained from the lateral branches
and the main stem of transgenic soybean plants transformed
with a seed specific expression cassette for CCP1 from
pMBXO75 compared to wild-type controls

| | Lateral branches | | Main Stem | | Total Plant | |
|---|---|---|---|---|---|---|
| | Seed | | Seed | | Seed | |
| | weight | % of | weight | % of | weight | % of |
| Line | (g) | control* | (g) | control* | (g) | control* |
| A6 | 19.053 | 137 | 11.952 | 78 | 31.005 | 106 |
| A8 | 25.89 | 186 | 5.708 | 37 | 31.598 | 108 |
| A11 | 19.177 | 138 | 11.627 | 76 | 30.804 | 106 |
| A12 | 20.937 | 150 | 12.559 | 82 | 33.496 | 115 |
| B11 | 22.954 | 165 | 12.942 | 85 | 35.896 | 123 |
| B12 | 15.527 | 112 | 6.969 | 46 | 22.496 | 77 |
| D12 | 9.643 | 69 | 8.334 | 55 | 17.977 | 62 |
| G2 | 19.309 | 139 | 14.016 | 92 | 33.325 | 114 |
| H3 | 22.207 | 159 | 15.1 | 99 | 37.307 | 128 |
| WT | 13.925 ± | 100 | 15.243 ± | 100 | 29.168 ± | 100 |
| | 4.502 | | 1.100 | | 4.985 | |

*% of control is calculated from the average of 6 wild-type control plants (Westag cultivar).
WT, wild-type

TABLE 14
Average individual seed weight of seeds obtained from lateral
branches or the main stem of transgenic soybean plants transformed
with a seed specific expression cassette for CCP1 from pMBXO75
compared to wild-type controls

| | Lateral Branches | | Main Stem | |
|---|---|---|---|---|
| | Avg individual | % to wild- | Avg individual | % to wild- |
| Line | seed weight (g) | type control* | seed weight (g) | type control* |
| A6 | 0.219 | 102 | 0.221 | 94 |
| A8 | 0.225 | 105 | 0.204 | 87 |
| A11 | 0.226 | 105 | 0.228 | 97 |
| A12 | 0.228 | 106 | 0.228 | 97 |
| B11 | 0.230 | 107 | 0.231 | 98 |
| B12 | 0.216 | 100 | 0.218 | 93 |
| D12 | 0.201 | 94 | 0.208 | 89 |
| G2 | 0.210 | 98 | 0.226 | 96 |
| H3 | 0.222 | 103 | 0.225 | 96 |
| WT | 0.215 ± 0.011 | 100 | 0.235 ± 0.011 | 100 |

*% of control is calculated from the average of 6 wild-type control plants (Westag cultivar).
WT, wild-type

Oil content of the seeds is measured after crushing seeds using standard procedures for preparation of fatty acid methyl esters as previously described for Camelina seeds by Malik et al. (Plant Biotechnology Journal, 2015, 13, 675) and for Arabidopsis seeds by Li et al. (Phytochemistry, 2006, 67, 904).

The best lines were picked to plant in soil to obtain T3 seeds for analysis.

### Example 8

### Co-Expression of Cassettes for CCP1 Containing Seed Specific and Constitutive Promoters

Producing soybean plants to combine the positive effects of seed specific and constitutive expression of CCP1 on yield increase in soybean is desired. This can be seen upon examination of the increase in lateral branches, pods, and numbers of seeds (Tables 15 and 16) in soybean plant transformed with a cassette containing the 4×35S constitutive promoter driving the expression of CCP1. More branches, pods, and seeds were produced however the seeds were of a smaller size. Smaller seeds have previously been observed in experiments with constitutive expression of CCP1 in Camelina sativa (Schnell et al., WO 2015/103074).

TABLE 15
Distribution of pods on transgenic soybean plants
transformed with a constitutive expression cassette
for CCP1 compared to wild-type controls

| Line | # of lateral branches | % to wild-type control* |
|---|---|---|
| N6 | 10 | 154 |
| WT | 6.50 ± 1.05 | 100 |

*% of control is calculated from the average of 6 wild-type control plants (Westag cultivar).
WT, wild-type

TABLE 16
Pod and seed production with expression of CCP1 in soybean

| | Lateral branches | | Main Stem | | Total Plant | |
|---|---|---|---|---|---|---|
| | | % of | | % of | | % of |
| Line | # pods | control* | # pods | control* | # pods | control* |
| N6 | 113 | 387 | 42 | 161 | 155 | 280 |
| WT | 29.17 ± 7.44 | 100 | 26.17 ± | 100 | 55.33 ± 6.41 | 100 |
| | | | 2.14 | | | |

| | Lateral branches | | Main Stem | | Total Plant | |
|---|---|---|---|---|---|---|
| | | % of | | % of | | % of |
| Line | # seeds | control* | # seeds | control* | #seeds | control* |
| N6 | 166 | 258 | 75 | 116 | 241 | 187 |
| WT | 64.33 ± 18.22 | 100 | 64.83 ± | 100 | 129.17 ± | 100 |
| | | | 4.54 | | 18.57 | |

| | Lateral branches | | Main Stem | | Total Plant | |
|---|---|---|---|---|---|---|
| | Harvested | | Harvested | | Harvested | |
| | seed | | seed | | seed | |
| | weight | % of | weight | % of | weight | % of |
| Line | (g) | control* | (g) | control* | (g) | control* |
| N6 | 26.0 | 187 | 11.964 | 78 | 37.964 | 130 |
| WT | 13.925 ± | 100 | 15.243 ± | 100 | 29.168 ± | 100 |
| | 4.502 | | 1.100 | | 4.985 | |

| | |
|---|---|
| Lateral Branches | Main Stem |

| | Avg individual | % of | Avg individual | % of |
|---|---|---|---|---|
| Line | seed weight (g) | control* | seed weight (g) | control* |
| N6 | 0.157 | 73 | 0.160 | 68 |
| WT | 0.215 | 100 | 0.235 | 100 |

*% of control is calculated from the average of 6 wild-type control plants (Westag cultivar).
WT, wild-type

### Example 9

### Seed Specific Expression of C. reinhardtii CCP1 in Rice

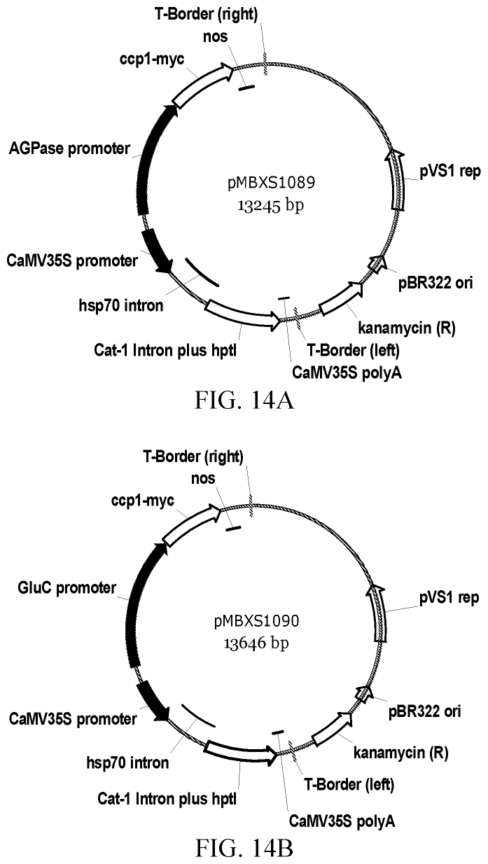

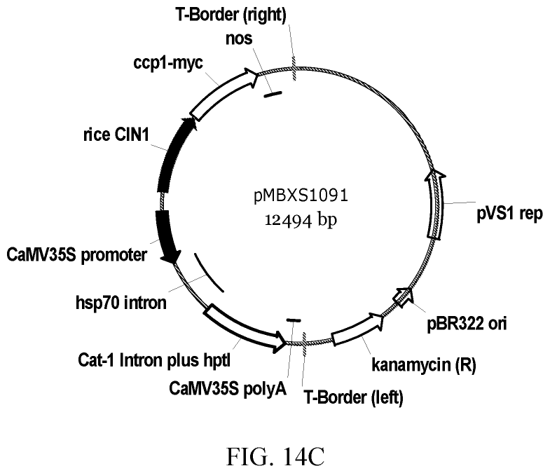

Several promoters were chosen for expression of the CCP1 gene in rice based on their experimental or in silico predicted expression profiles in rice seed. The promoter from the rice ADP-glucose pyrophosphorylase (AGPase) gene (GenBank: AY427566.1, LOC_Os01g44220) has been shown to be expressed in the seed as well as the phloem of vegetative tissues in rice (Qu, L. Q. and Takaiwa, F., 2004, Plant Biotechnology Journal, 2, 113-125). Plant transformation construct pMBXS1089 (FIG. 14A), contains an expression cassette with the AGPase promoter driving the expression of the CCP1 coding sequence. The CCP1 gene was fused at the C-terminus to a DNA fragment encoding a myc tag. The myc tag can allow detection or purification of the expressed CCP1-myc fusion protein using commercially available antibodies to the myc tag or purification kits. A second plant transformation construct, pMBXS1090 (FIG. 14B), was prepared using the promoter from the rice glutelin C (GluC) gene (GenBank: EU264107.1, LOC_Os02g25640) to drive expression of the CCP1-myc fusion. The GluC promoter has been shown to be expressed in the whole endosperm of rice seed (Qu, L. Q. et al., 2008, Journal of Experimental Biology, 59, 2417-2424). A third transformation construct pMBXS1091 (FIG. 14C) containing the promoter from the rice beta-fructofuranosidase insoluble isoenzyme 1 (CIN1) gene driving the expression of CCP1-myc was also prepared. The CIN1 promoter was chosen based on in silico expression data showing expression throughout various developmental stages but with highest expression in the inflorescence and seeds (Rice Genome Annotation Project; http://rice.plantbiology.msu.edu/cgi-bin/ORF_infopage.cgi?orf=LOC_Os02g33110.1).

In preparation for rice transformation, callus of the rice cultivar Nipponbare was initiated from mature, dehusked, surface sterilized seeds on N6-basal salt callus induction media (N6-CI; contains per liter 3.9 g CHU (N₆) basal salt mix [Sigma Catalog #C1416]; 10 ml of 100× N6-vitamins [contains in final volume of 500 mL, 100 mg glycine, 25 mg nicotinic acid, 25 mg pyridoxine hydrochloride and 50 mg thiamin hydrochloride]; 0.1 g myo-inositol; 0.3 g casamino acid (casein hydrolysate); 2.88 g proline; 10 ml of 100× 2,4-dichlorophenoxyacetic acid (2,4-D), 30g sucrose, pH 5.8 with 4 g gelrite or phytagel). Approximately 100 seeds were used for each transformation. The frequency of callus induction was scored after 21 days of culture in the dark at 27±1° C. Callus induction from the scutellum with a high frequency (of about 96% total callus induction) was observed.

Rice transformation vector pMBXS1091 was transformed into Agrobacterium strain AGL1. The resulting Agrobacterium strain was resuspended in 10 mL of MG/L medium (5 g tryptone, 2.4 g yeast extract, 5 g mannitol, 5 g Mg₂SO₄, 0.25 g K₂HPO₄, 1 g glutamic acid and 1 g NaCl) to a final OD600 of 0.3. Approximately twenty-one day old scutellar embryogenic callus were cut to about 2-3 mm in size and were infected with Agrobacterium containing pMBXS1091 for 5 min. After infection, the calli were blotted dry on sterile filter papers and transferred onto co-cultivation media (N6-CC; contains per liter 3.9 g CHU (N₆) basal salt mix; 10 ml of 100× N6-vitamins; 0.1 g myo-inositol; 0.3 g casamino acid; 10 ml of 100× 2,4-D, 30g sucrose, 10 g glucose, pH 5.2 with 4g gelrite or phytagel and 1 mL of acetosyringone [19.6 mg/mL stock]). Co-cultivated calli were incubated in the dark for 3 days at 25 ° C. After three days of co-cultivation, the calli were washed thoroughly in sterile distilled water to remove the bacteria. A final wash with a timentin solution (250 mg/L) was performed and calli were blotted dry on sterile filter paper. Callus were transferred to selection media (N6-SH; contains per liter 3.9 g CHU (N₆) basal salt mix, 10 ml of 100× N6-vitamins, 0.1 g myo-inositol, 0.3 g casamino acid, 2.88 g proline, 10 ml of 100×, 2,4-D, 30g sucrose, pH 5.8 with 4g phytagel and 500 μL of hygromycin (stock concentration: 100 mg/ml ) and incubated in the dark for two-weeks at 27±1° C. The transformed calli that survived the selection pressure and that proliferated on N6-SH medium were sub-cultured on the same media for a second round of selection. These calli were maintained under the same growth conditions for another two-weeks. The number of plants regenerated after 30 days on N6-SH medium was scored and the frequency calculated. After 30 days, the proliferating calli were transferred to regeneration media (N6-RH medium; contains per liter 4.6 g MS salt mixture, 10 ml of 100× MS-vitamins [MS-vitamins contains in 500 mL final volume 250 mg nicotinic acid, 500 mg pyridoxine hydrochloride, 500 mg thiamine hydrochloride, 100 mg glycine], 0.1 g myo-inositol, 2 g casein hydrolysate, 1 ml of 1,000× 1-naphtylacetic acid solution [NAA; contains in 200 mL final volume 40 mg NAA and 3 mL of 0.1 N NaOH], 20 ml of 50× kinetin [contains in 500 mL final volume 50 mg kinetin and 20 mL 0.1 N HCl], 30g sucrose, 30g sorbitol, pH 5.8 with 4g phytagel and 500 μl of a 100 mg/mL hygromycin stock). The regeneration of plantlets from these calli occurred after about 4-6 weeks. Rooted plants were transferred into peat-pellets for one week to allow for hardening of the roots. The plants were then kept in zip-loc bags for acclimatization. Plants were transferred into pots and grown in a greenhouse to maturity. The number of tillers and panicles from each transgenic plants was counted and compared to the wild-type controls (TABLE 17).

TABLE 17
Comparison of number of tillers and panicles produced
in primary transformants of transgenic rice transformed
with pMBXS1091 compared with wild-type controls.

| | Tillers | | Panicles | |
|---|---|---|---|---|
| | | % to highest | | % to highest |
| | | wild-type | | wild-type |
| Line | Number | control¹ | Number | control² |
| NB-E | 29 | | 26 | 100 |
| (wild-type) | | | | |
| NB-D | 36 | 100 | 22 | |
| (wild-type) | | | | |
| NB-C | 24 | | 0 | |
| (wild-type) | | | | |
| P1091-8B | 81 | 225 | 48 | 185 |
| P1091-9B | 51 | 142 | 43 | 165 |
| P1091-2C | 61 | 169 | 38 | 146 |
| P1091-8A | 48 | 133 | 35 | 135 |
| P1091-1A | 53 | 147 | 32 | 123 |
| P1091-8C | 51 | 142 | 30 | 115 |
| P1091-11C | 43 | 119 | 29 | 112 |
| P1091-2A | 32 | | 29 | 112 |
| P1091-2B | 35 | | 29 | 112 |
| P1091-6A | 48 | 133 | 28 | 108 |
| P1091-9D | 36 | 100 | 28 | 108 |
| P1091-10F | 57 | 158 | 27 | 104 |
| P1091-11B | 42 | 117 | 27 | 104 |
| P1091-4A | 48 | 133 | 27 | 104 |
| P1091-4B | 34 | | 27 | 104 |
| P1091-3B | 29 | | 26 | 100 |
| P1091-7A | 71 | 197 | 26 | 100 |
| P1091-10E | 30 | | 24 | |
| P1091-12B | 31 | | 24 | |
| P1091-11A | 32 | | 23 | |
| P1091-9A | 43 | 119 | 21 | |
| P1091-10A | 34 | | 20 | |
| P1091-1D | 24 | | 17 | |
| P1091-2E | 39 | 108 | 16 | |
| P1091-2D | 36 | 100 | 10 | |
| P1091-4C | 58 | 161 | 9 | |
| P1091-1B | 23 | | 7 | |
| P1091-1E | 19 | | 7 | |
| P1091-5A | 28 | | 6 | |
| P1091-9C | 45 | 125 | 4 | |
| P1091-10C | 63 | 175 | 0 | |
| P1091-10D | 46 | 128 | 0 | |
| P1091-4D | 33 | | 0 | |
| P1091-5C | 31 | | 0 | |

¹The % to wild-type control was calculated using the best wild-type plant that produced the most tillers. Only % to control values equal or greater than 100% are shown.
²The % to wild-type control was calculated using the best wild-type plant that produced the most panicles. Only % to control values equal or greater than 100% are shown.

Seed is harvest from each panicle (Ti generation) and the seed yield per plant is calculated.

T1 seed is grown in a greenhouse to produce T2 seed. The mass of the total seed per plant is collected to compare seed yield of transgenics to wild-type control plants.

The transformation is repeated with constructs pMBXS1089 and pMBXS1090.

### Example 10

### Methods for Characterizing CCP1 Transport

Some mitochondrial and plastid carrier proteins have previously been shown to functionally localize into the E. coli cytoplasmic membrane including mitochondrial ADP/ATP carriers (Haferkamp et al. (2002), European Journal of Biochemistry 269, 3172; Razakantoanina, et al. (2008), Experimental Parasitology 118, 181), plastid ATP/ADP transporter genes (Tjaden, et al. (1998), J Biol Chem 273, 9630), and some bicarbonate transporters (Du et al. (2014), PLoS One 9, e115905).

Cyanobacterial bicarbonate transporters have been characterized in Escherichia coli using a mutant E. coli strain, termed EDCM636, that is deficient in carbonic anhydrase activity (Du, J. et al. (2014)). This mutant is unable to grow on LB or M9 plates without supplementation with high levels of CO₂. As reported by Du et al. (2014), expression of six cyanobacterial bicarbonate transporters, corresponding to β forms of SbtA of Synechococcus sp. WH5701, Cyanobium sp. PCC 7001, Cyanobium sp. PCC 6307, Synechococcus elongatus PCC 7942, Synechocystis sp. PCC 6803, and Synechococcus sp. PCC 7002, restored growth of the E. coli mutant at atmospheric levels of CO₂, whereas expression of various others did not.

The function of CCP1 and potential orthologs thereof with respect to bicarbonate or other small molecule transport may be tested by an analogous approach, and corresponding functional screens developed, also based on restoring growth of a mutant E. coli strain that is deficient in an enzymatic activity that prevents that production of a small molecule required for growth. For example, the CCP1 coding sequence from Chlamydomonas reinhardtii can be synthesized with a sequence that is codon optimized for expression in E. coli and cloned into an E. coli expression vector. Codon optimized sequences of potential orthologs thereof can also can be synthesized and cloned into E. coli expression vectors.

For testing bicarbonate transport, codon optimized sequences of two SbtA bicarbonate transporters from Cyanobium sp. PCC 7001 (also termed SbtA_{Cyanobium sp.pCC 7001}) and Synechocystis sp. PCC 6803 (also termed SbtA_{Synechocystis sp.PCC 6803}) can be synthesized and cloned into E. coli expression vectors. These two SbtA proteins can serve as positive controls for functional heterologous expression in E. coli, based on SbtA of Cyanobium sp. PCC 7001 having a Kₘ calculated to be 189 μM and SbtA of Synechocystis sp. PCC 6803 having a Kₘ under 100 μM, and based on both previously having been shown to enable E. coli bicarbonate uptake, as taught by Du et al. The E. coli expression vector lacking a cloned sequence can serve as a negative control. Restoration of growth of the mutant E. coli strain by the CCP1 coding sequence and by potential orthologs thereof would indicate that these sequences also enable E. coli bicarbonate uptake.

Likewise, E. coli mutants deficient in the transport and/or production of small molecules, such as for example C4-dicarboxylic acids, can be used to test the ability of CCP1 to transport α-ketoglutarate, succinate, malate, and oxaloacetate. The ychM gene of E. coli has been shown to be the main succinate transporter under acidic pH growth conditions (Karinou et al., 2013, Molecular Microbiology, 87, 623) and an E. coli strain with a mutated ychM gene can be used to characterize the ability of CCP1 to transport this molecule.

Function of CCP1 and potential orthologs thereof with respect to bicarbonate transport also may be tested, and corresponding functional screens developed, based on use of yeast modified to express CCP1 and potential orthologs thereof. For example, a functional screen for CCP1 expression in yeast based on sensitivity of growth to bicarbonate works as follows. CCP1 can be expressed in yeast to examine if CCP1 utilizes HCO₃⁻ as a substrate. HCO₃⁻ is the major pH regulator of the yeast cytosol. Accordingly, disruptions in regulation of HCO₃⁻ at the mitochondrial membrane result in a loss of respiration and an inhibition of growth. Increasing concentrations of HCO₃⁻in media should result in rapid inhibition of yeast growth in cultures expressing CCP1 relative to yeast transformed with an empty vector control. Non-specific compounds, such as borate, NaCl and nitrate, also can be used as negative controls, as these would not be expected to inhibit growth. In accordance with this approach, function of CCP1 and/or other mitochondrial transporter proteins of eukaryotic algae that are orthologs of CCP1 as transporter proteins can be confirmed. Moreover, additional mitochondrial transporter proteins that are localized to mitochondria and that function similarly can be identified.

### Example 11

### Model for Further Enhanced Yield of Plants Based on Inhibiting Expression of CWII that Would Otherwise be Upregulated in CCP1 Lines

A model for further enhanced yield based on inhibiting expression of cell wall invertase inhibitor that would otherwise be upregulated in CCP1 lines is provided, with reference to FIG. 15, as follows.

It is believed that expression of a novel class of cell wall invertase inhibitors is upregulated in plants modified to express CCP1 of Chlamydomonas reinhardtii and/or other mitochondrial transporter proteins of eukaryotic algae that are orthologs of CCP1. In accordance with this model, sucrose transport and allocation is a key determinant of seed yield. Export and import of sucrose through the apoplasm are controlled by cell wall invertases (also termed CWI), which hydrolyze sucrose to fructose and glucose. Activity of cell wall invertase is controlled by a cell wall invertase inhibitor. The novel class of cell wall invertase inhibitors is upregulated in plants modified to express CCP1 of Chlamydomonas reinhardtii. This is likely a response of cells to increased carbon capture. Also, cell wall invertase inhibitors are good targets for genome editing. Accordingly, it is believed that downregulating cell wall invertase inhibitor genes in plants modified to express CCP1 of Chlamydomonas reinhardtii and/or other mitochondrial transporter proteins of eukaryotic algae that are orthologs of CCP1 would result in further enhanced yield.

### Exemplary Embodiments

The following are exemplary embodiments of the transgenic land plants comprising a mitochondrial transporter protein of a eukaryotic algae as disclosed herein.

Embodiment A. A transgenic land plant comprising a mitochondrial transporter protein of a eukaryotic algae, wherein:

the mitochondrial transporter protein of the eukaryotic algae is heterologous with respect to the transgenic land plant;

the mitochondrial transporter protein corresponds to a sequence or ortholog of (a) CCP1 of Chlamydomonas reinhardtii of SEQ ID NO: 1, (b) a mitochondrial transporter protein of Chlorella sorokiniana of SEQ ID NO: 2, (c) a mitochondrial transporter protein of Chlorella variabilis of SEQ ID NO: 3, SEQ ID NO: 4, SEQ ID NO: 5, or SEQ ID NO: 6, (d) a mitochondrial transporter protein of Chondrus crispus of SEQ ID NO: 7, SEQ ID NO: 8, or SEQ ID NO: 9, (e) a mitochondrial transporter protein of Gonium pectorals of SEQ ID NO: 19, or SEQ ID NO: 20, or (f) a mitochondrial transporter protein of Volvox carteri of SEQ ID NO: 21;

the mitochondrial transporter protein is localized to mitochondria of the transgenic land plant based on a mitochondrial targeting signal intrinsic to the mitochondrial transporter protein; and

the mitochondrial transporter protein is expressed predominantly in seeds of the transgenic land plant.

Embodiment B. The transgenic land plant of embodiment A, wherein the mitochondrial transporter protein corresponds to a mitochondrial transporter protein selected from the group consisting of (a) CCP1 of Chlamydomonas reinhardtii of SEQ ID NO: 1; (b) a mitochondrial transporter protein of Chlorella sorokiniana of SEQ ID NO: 2, (c) a mitochondrial transporter protein of Chlorella variabilis of SEQ ID NO: 3, SEQ ID NO: 4, SEQ ID NO: 5, or SEQ ID NO: 6, (d) a mitochondrial transporter protein of Chondrus crispus of SEQ ID NO: 7, SEQ ID NO: 8, or SEQ ID NO: 9, (e) a mitochondrial transporter protein of Gonium pectorals of SEQ ID NO: 19, or SEQ ID NO: 20, and (f) a mitochondrial transporter protein of Volvox carteri of SEQ ID NO: 21.

Embodiment C. The transgenic land plant of embodiments A or B, wherein the mitochondrial transporter protein is an ortholog of CCP1 of Chlamydomonas reinhardtii of SEQ ID NO: 1 based on comprising: (i) (a) a proline residue at position 268, (b) an aspartate residue or glutamine residue at position 270, (c) a lysine residue or arginine residue at position 273, and (d) a serine residue or threonine residue at position 274, with numbering of positions relative to CCP1 of Chlamydomonas reinhardtii of SEQ ID NO: 1, and (ii) an overall identity of at least 15%.

Embodiment D. The transgenic land plant of any one of embodiments A-C, wherein the mitochondrial transporter protein is an ortholog of CCP1 of Chlamydomonas reinhardtii of SEQ ID NO: 1 based on comprising: (i) (a) a glycine residue at position 301, (b) a glycine residue at position 308, and (c) an arginine residue at position 315, with numbering of positions relative to CCP1 of Chlamydomonas reinhardtii of SEQ ID NO: 1, and (ii) an overall identity of at least 15%.

Embodiment E. The transgenic land plant of any one of embodiments A-D, wherein the mitochondrial transporter protein is localized to mitochondria of the transgenic land plant to a greater extent than to chloroplasts of the transgenic land plant by a factor of at least 2, at least 5, or at least 10.

Embodiment F. The transgenic land plant of any one of embodiments A-E, wherein the mitochondrial transporter protein consists essentially of an amino acid sequence that is identical to that of a wild-type eukaryotic algal mitochondrial transporter protein.

Embodiment G. The transgenic land plant of any one of embodiments A-F, further comprising a heterologous polynucleotide, wherein the mitochondrial transporter protein is encoded by the heterologous polynucleotide.

Embodiment H. The transgenic land plant of embodiment G, wherein the heterologous polynucleotide comprises a heterologous promoter.

Embodiment I. The transgenic land plant of embodiment H, wherein the heterologous promoter is a seed-specific promoter.

Embodiment J. The transgenic land plant of any of embodiments G-I, wherein the heterologous polynucleotide is integrated into genomic DNA of the transgenic land plant.

Embodiment K. The transgenic land plant of any of embodiments A-J, wherein the transgenic land plant (i) expresses the mitochondrial transporter protein in a seed-specific manner, and (ii) expresses another mitochondrial transporter protein constitutively, the other mitochondrial transporter protein also corresponding to a sequence or ortholog of (a) CCP1 Chlamydomonas reinhardtii of SEQ ID NO: 1, (b) a mitochondrial transporter protein of Chlorella sorokiniana of SEQ ID NO: 2, (c) a mitochondrial transporter protein of Chlorella variabilis of SEQ ID NO: 3, SEQ ID NO: 4, SEQ ID NO: 5, or SEQ ID NO: 6, (d) a mitochondrial transporter protein of Chondrus crispus of SEQ ID NO: 7, SEQ ID NO: 8, or SEQ ID NO: 9, (e) a mitochondrial transporter protein of Gonium pectorals of SEQ ID NO: 19, or SEQ ID NO: 20, or (f) a mitochondrial transporter protein of Volvox carteri of SEQ ID NO: 21.

Embodiment L. The transgenic land plant of any of embodiments A-K, wherein the transgenic land plant has a CO₂ assimilation rate that is at least 5% higher, at least 10% higher, at least 20% higher, or at least 40% higher, than for a corresponding reference land plant not comprising the mitochondrial transporter protein.

Embodiment M. The transgenic land plant of any of embodiments A-L, wherein the transgenic land plant has a transpiration rate that is at least 5% lower, at least 10% lower, at least 20% lower, or at least 40% lower, than for a corresponding reference land plant not comprising the mitochondrial transporter protein.

Embodiment N. The transgenic land plant of any of embodiments A-M, wherein the transgenic land plant has a seed yield that is at least 5% higher, at least 10% higher, at least 20% higher, at least 40% higher, at least 60% higher, or at least 80% higher, than for a corresponding reference land plant not comprising the putative mitochondrial transporter protein.

Embodiment O. The transgenic land plant of any embodiments A-N, wherein the transgenic land plant is modified to express (i) a suppressor of an endogenous cell wall invertase inhibitor of the transgenic land plant or (ii) a modified cell wall invertase inhibitor in place of an endogenous cell wall invertase inhibitor of the transgenic land plant.

Embodiment P. The transgenic land plant of embodiment 0, wherein the suppressor of the endogenous cell wall invertase inhibitor is (i) an antisense RNA complementary to messenger RNA of the endogenous cell wall invertase inhibitor or (ii) an RNA interference nucleic acid that reduces expression of messenger RNA of the endogenous cell wall invertase inhibitor.

Embodiment Q. The transgenic land plant of embodiment 0, wherein the modified cell wall invertase inhibitor has been modified by transforming the transgenic land plant with a nucleotide sequence encoding CRISPR-associated protein 9 under the control of a promoter and with a nucleotide sequence encoding a single guide RNA under the control of a promoter, wherein the single guide RNA comprises 19 to 22 nucleotides and is fully homologous to a region of a gene encoding the endogenous cell wall invertase inhibitor.

Embodiment R. The transgenic land plant of any of embodiments A-N, wherein the transgenic land plant is modified to express carbonic anhydrase targeted to mitochondria.

Embodiment S. The transgenic land plant of embodiment R, wherein the carbonic anhydrase is a carbonic anhydrase of rice, maize, soybean, canola, camelina, tomato, barley, cucumber, alfalfa, bean, pea, pear, almond, or mung bean that is targeted to mitochondria.

Embodiment T. The transgenic land plant of embodiment R, wherein the carbonic anhydrase is a carbonic anhydrase of tobacco, cotton, aspen, or Arabidopsis that is targeted to mitochondria.

Embodiment U. The transgenic land plant of embodiment R, wherein the carbonic anhydrase is a carbonic anhydrase of a eukaryotic algae that is targeted to mitochondria.

Embodiment V. The transgenic land plant of any of embodiments A-N, wherein the only heterologous algal protein that the transgenic land plant comprises is the mitochondrial transporter protein.

Embodiment W. The transgenic land plant of any of embodiments A-V, wherein the transgenic land plant is a C3 plant.

Embodiment X. The transgenic land plant of any of embodiments A-V, wherein the transgenic land plant is a C4 plant.

Embodiment Y. The transgenic land plant of any of embodiments A-V, wherein the transgenic land plant is a food crop plant selected from the group consisting of maize, rice, wheat, oat, barley, soybean, millet, sorghum, potato, pulse, bean, and tomato.

Embodiment Z. The transgenic land plant of any of embodiments A-V, wherein the transgenic land plant is a forage crop plant selected from the group consisting of hay, alfalfa, and silage corn.

Embodiment AA. The transgenic land plant of any of embodiments A-V, wherein the transgenic land plant is an oilseed crop plant selected from the group consisting of camelina, Brassica species (e.g. B. napus (canola), B. rapa, B. juncea, and B. carinata), crambe, soybean, sunflower, safflower, oil palm, flax, and cotton.

The invention has been described with reference to the example embodiments described above. Modifications and alterations will occur to others upon a reading and understanding of this specification. Examples embodiments incorporating one or more aspects of the invention are intended to include all such modifications and alterations insofar as they come within the scope of the appended claims.

### REFERENCE TO A “SEQUENCE LISTING,” A TABLE, OR A COMPUTER PROGRAM LISTING APPENDIX SUBMITTED AS AN ASCII TEXT FILE

The material in the ASCII text file, named “YTEN-57171WO-Sequences ST25.txt”, created Feb. 7, 2018, file size of 180,224 bytes, is hereby incorporated by reference.

1. A transgenic land plant comprising a mitochondrial transporter protein of a eukaryotic algae, wherein:
the mitochondrial transporter protein of the eukaryotic algae is heterologous with respect to the transgenic land plant;
the mitochondrial transporter protein corresponds to a sequence or ortholog of (a) CCP1 of Chlamydomonas reinhardtii of SEQ ID NO: 1, (b) a mitochondrial transporter protein of Chlorella sorokiniana of SEQ ID NO: 2, (c) a mitochondrial transporter protein of Chlorella variabilis of SEQ ID NO: 3, SEQ ID NO: 4, SEQ ID NO: 5, or SEQ ID NO: 6, (d) a mitochondrial transporter protein of Chondrus crispus of SEQ ID NO: 7, SEQ ID NO: 8, or SEQ ID NO: 9, (e) a mitochondrial transporter protein of Gonium pectorals of SEQ ID NO: 19, or SEQ ID NO: 20, or (f) a mitochondrial transporter protein of Volvox carteri of SEQ ID NO: 21;
the mitochondrial transporter protein is localized to mitochondria of the transgenic land plant based on a mitochondrial targeting signal intrinsic to the mitochondrial transporter protein; and
the mitochondrial transporter protein is expressed predominantly in seeds of the transgenic land plant.

2. The transgenic land plant of claim 1, wherein the mitochondrial transporter protein corresponds to a mitochondrial transporter protein selected from the group consisting of (a) CCP1 of Chlamydomonas reinhardtii of SEQ ID NO: 1; (b) a mitochondrial transporter protein of Chlorella sorokiniana of SEQ ID NO: 2, (c) a mitochondrial transporter protein of Chlorella variabilis of SEQ ID NO: 3, SEQ ID NO: 4, SEQ ID NO: 5, or SEQ ID NO:
6, (d) a mitochondrial transporter protein of Chondrus crispus of SEQ ID NO: 7, SEQ ID NO: 8, or SEQ ID NO: 9, (e) a mitochondrial transporter protein of Gonium pectorale of SEQ ID NO:
19, or SEQ ID NO: 20, and (f) a mitochondrial transporter protein of Volvox carteri of SEQ ID NO: 21.

3. The transgenic land plant of claim 1, wherein the mitochondrial transporter protein is an ortholog of CCP1 of Chlamydomonas reinhardtii of SEQ ID NO: 1 based on comprising: (i) (a) a proline residue at position 268, (b) an aspartate residue or glutamine residue at position 270, (c) a lysine residue or arginine residue at position 273, and (d) a serine residue or threonine residue at position 274, with numbering of positions relative to CCP1 of Chlamydomonas reinhardtii of SEQ ID NO: 1, and (ii) an overall identity of at least 15%.

4. The transgenic land plant of claim 1, wherein the mitochondrial transporter protein is an ortholog of CCP1 of Chlamydomonas reinhardtii of SEQ ID NO: 1 based on comprising: (i) (a) a glycine residue at position 301, (b) a glycine residue at position 308, and (c) an arginine residue at position 315, with numbering of positions relative to CCP1 of Chlamydomonas reinhardtii of SEQ ID NO: 1, and (ii) an overall identity of at least 15%.

5. The transgenic land plant of claim 1, wherein the mitochondrial transporter protein is localized to mitochondria of the transgenic land plant to a greater extent than to chloroplasts of the transgenic land plant by a factor of at least 2.

6. (canceled)

7. The transgenic land plant of claim 1, further comprising a heterologous polynucleotide, wherein the mitochondrial transporter protein is encoded by the heterologous polynucleotide.

8. The transgenic land plant of claim 7, wherein the heterologous polynucleotide comprises a heterologous promoter.

9. The transgenic land plant of claim 8, wherein the heterologous promoter is a seed-specific promoter.

10. The transgenic land plant of claim 7, wherein the heterologous polynucleotide is integrated into genomic DNA of the transgenic land plant.

11. Cancelled

12. The transgenic land plant of claim 1, wherein the transgenic land plant has a CO₂ assimilation rate that is at least 5% higher than for a corresponding reference land plant not comprising the mitochondrial transporter protein.

13. The transgenic land plant of claim 1, wherein the transgenic land plant has a transpiration rate that is at least 5% lower than for a corresponding reference land plant not comprising the mitochondrial transporter protein.

14. The transgenic land plant of claim 1, wherein the transgenic land plant has a seed yield that is at least 5% higher than for a corresponding reference land plant not comprising the putative mitochondrial transporter protein.

15. The transgenic land plant of claim 1, wherein the transgenic land plant is modified to express (i) a suppressor of an endogenous cell wall invertase inhibitor of the transgenic land plant or (ii) a modified cell wall invertase inhibitor in place of an endogenous cell wall invertase inhibitor of the transgenic land plant.

16. The transgenic land plant of claim 15, wherein the suppressor of the endogenous cell wall invertase inhibitor is (i) an antisense RNA complementary to messenger RNA of the endogenous cell wall invertase inhibitor or (ii) an RNA interference nucleic acid that reduces expression of messenger RNA of the endogenous cell wall invertase inhibitor.

17. The transgenic land plant of claim 15, wherein the modified cell wall invertase inhibitor has been modified by transforming the transgenic land plant with a nucleotide sequence encoding CRISPR-associated protein 9 under the control of a promoter and with a nucleotide sequence encoding a single guide RNA under the control of a promoter, wherein the single guide RNA comprises 19 to 22 nucleotides and is fully homologous to a region of a gene encoding the endogenous cell wall invertase inhibitor.

18. The transgenic land plant of claim 1, wherein the transgenic land plant is modified to express carbonic anhydrase targeted to mitochondria.

19. The transgenic land plant of claim 18, wherein the carbonic anhydrase is a carbonic anhydrase of rice, maize, soybean, canola, camelina, tomato, barley, cucumber, alfalfa, bean, pea, pear, almond, or mung bean that is targeted to mitochondria.

20. The transgenic land plant of claim 18, wherein the carbonic anhydrase is a carbonic anhydrase of tobacco, cotton, aspen, or Arabidopsis that is targeted to mitochondria.

21. The transgenic land plant of claim 18, wherein the carbonic anhydrase is a carbonic anhydrase of a eukaryotic algae that is targeted to mitochondria.

22-26. (canceled)

27. The transgenic land plant of claim 1, wherein the transgenic land plant is an oilseed crop plant selected from the group consisting of camelina, Brassica species (e.g. B. napus (canola), B. rapa, B. juncea, and B. carinata), crambe, soybean, sunflower, safflower, oil palm, flax, and cotton.


SEQUENCE LISTING (21 sequences)

SEQ ID NO: 1 - protein, 358 aa, Chlamydomonas reinhardtii
MSSDAMTINESLMEVEHTPAVHKRILDILPGISGGVARVMIGQPFDTIKVRLQVLGQGTALAAKLPPSEVYKDSMDCIRKMIKSEGPLSFYKGTVAPLVGNMVLLGIHFPVFSAVRKQLEGDDHYSNFSHANVLLSGAAAGAAGSLISAPVELVRTKMQMQRRAALAGTVAAGAAASAGAEEFYKGSLDCFKQVMSKHGIKGLYRGFTSTILRDMQGYAWFFLGYEATVNHFLQNAGPGVHTKADLNYLQVMAAGVVAGFGLWGSMFPIDTIKSKLQADSFAKPQYSSTMDCLKKVLASEGQAGLWRGFSAAMYRAIPVNAGIFLAVEGTRQGIKWYEENVEHIYGGVIGPATPTAAQ

SEQ ID NO: 2 - protein, 353 aa, Chlorella sorokiniana
MVARTINETLMEVEHTPPVHKRVLDVLPGVSGGVTRVLVGQPFDTIKTRLQVMGQGTALAKMLPPSDVYINSSDCLKKMVRNEGALSLYRGVVAPLLGNMVLLGIHFPTFSNTRKYLESVDATPAGEFPYWKVLAAGGAAGLAGSFISCPSEHIRTKMQLQRRAALAAQMGLKAQGLETYKGSWDCAVQILRNHGIKGLYRGMTSTVLRDIQGYAWFFLCYEATLHALAGPAHTRSELDYKHVLGAGVMAGFGLWGSMFPIDTIKSKMQGDSLSNPQYRNTLDCLRQSVAVEGFGGLFRGFGAAMYRAIPVNAGIFLAVEGTRQLLNKYEGYIDEKLGISVPASAATVPAPAQ

SEQ ID NO: 3 - protein, 306 aa, Chlorella-like algae
MPHNETTPAALPFYKTFAASAAAACTGEVATIPMDTVKVRLQVQGASGAPAKYKGTLGTLAKVAREEGVASLYKGLVPGLHRQILLGGVRIATYDPIRDFYGRLMKEEAGHTSIPTKIAAALTAGTFGVLVGNPTDVLKVRMQAQGKLPAGTPSRYPSAMAAYGMIVRQEGVKALWTGTTPNIARNSVVNAAELATYDQIKQLLMASFGFHDNVYCHLSASLCAGFLAVAAGSPFDVIKSRAMALSATGGYQGVGHVVMQTMRNEGLLAFWSGFSANFLRLGSWNIAMFLTLEKLRHLMGAPSAKH

SEQ ID NO: 4 - protein, 323 aa, Chlorella-like algae
MQEIQMPAVPAPPTLAAPQPASGFVRFAKDSFAGTVGGIAVTMVGHPFDTVKVRLQTQPSVNPIYNGAIDCVKKTLQWEGVPGLYKGVTSPLAGQMFFRATLFSAFGASKRWLGTNADGTTRDLTTADYYKAGFITGAAAAFTEAPIDFYKSQIQVQMVRAKADPTYKAPYTSVGECIKATVRYSGFKAPFQGLSATLLRNAPANAIYLGSFEVLKQQASKYYGCAPKDLSAPVVMAAGGTGGILYWLAIFPVDVIKSAMMTDSIDPAQRKYPTIPSTAKALWAEGGLSRFYRGFSPCIMRAAPANAVMLFTVDRVSHLLSDH

SEQ ID NO: 5 - protein, 323 aa, Chlorella-like algae
MTAGKSGLHPAADYVAGAIAGSANIALGFPADTVKVRLQNRLNPYNGAWHCATSMLRNEGARSLYRGMSPQLVGGAVETGVNYAVYQAMLGLTQGPRLALPEAAAVPLSAAAAGAVLSVVLSPAELVKCRLQLGGTERYHSYRGPVDCLRQTVQQEGLRGLMRGLSGTMAREIPGNAIYFSTYRLLRYWVSGGDPAATAAAASGATVAAASQPRSLLAFLVDSASAVVCGGLAGMVMWAAVLPLDVAKTRIQTAYPGSYQDVGVARQLHMVYREGGIQALYAGLSPTLARAFPANAAQWLAWELCMQQMQQWGGGGGRGGSST

SEQ ID NO: 6 - protein, 303 aa, Chlorella-like algae
MRTGVAVDLASGTAAGAAQLLVGHPFDTIKVNMQVGSADTTAMGAARRIVGTHGPLGMYRGLAAPLATVAAFNAVLFSSWGATERMLSPDGGCCPLTVGQAMLAGGLAGVPVSLLATPTELLKCRLQAQGGARPPPGMVYSLADIRAGRALFNGPLDVLRHVVRHEGGWLGAYRGLGATLLREVPGNAAYFGVYEGCKYGLARWQCIPTSELGPASLMTAGGVGGAAFWIVTYPFDVVKSRLQTQNIHALDRYHGTWDCMTRLYSAQGWQALWRGFGPCMARSVPANAVAFLAFEQVRAALSH

SEQ ID NO: 7 - protein, 328 aa, Chondrus crispus
MPSTTPLVDATSPAAATPDASATAVPAPVSIAAAAGPVYPPYAHALAGAGGGLATVTLLHPLDTLRTRLQSVERRAVLARRGDAVRAFKEILVREGAPALYRGVVPAAFGSVLSWACYFHWFQRARTIVKPAITHETGSHLLAGTIAGLMTSFATNPIWVVKVRLQLQRTGKSVAPGFKPYSGFFDGLKSITREEGVRGLYRGIGPSVWLVSHGAVQFTMYERFKERLRQDADPQSGTTVFHSLIASTGSKLVASLATYPLQVARTRMQERFADGRRYGNFHTAFMYIFRTEGIRGLYRGLSANVIRVTPQAAVTFITYEQILKLCAN

SEQ ID NO: 8 - protein, 233 aa, Chondrus crispus
VSREGAAGLYAGIQAPLPFVAVFNATLFAANSTMRKVVGKGRPDDDLSIAQIGLAGAGAGAAVSFVACPTELVKCRLQAQPGAFNGAIDCTRQVVANRGMGGLFTGMGATMVREMPGNALMFMTYNATMRALCSPGQATKDLSASQLMFAGGMAGLAFWMPCYPIDFAKTLIQTDSETNPRYRGLLDCMRKTVKAEGVGGLYKGIGPCLARAVPANAVTFLIYQWTLQLLGHS

SEQ ID NO: 9 - protein, 194 aa, Chondrus crispus
MGRPDDDLSIAQIGLAGAGAGPAVSFVACPTELIKCRLQAQPGAFNGAIDCTRQVVANRGMGGLFTGMGATMVREMPGNALMFMTYNATMRALCSPGQATKDLSASQLMFAGGMACLAFWMPCYPIDFAKTLIQTDSETNPRYRGLLDCMRKTVKAEGVGGLYKGIGPCLARAVPANAVTFLIDQCTLQLLGHS

SEQ ID NO: 10 - DNA, 11031 bp, Artificial Sequence
  pMBXO85
tcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgaaaatggttgcaagaaccattaacgagactcttatggaagtggaacatacaccgccagtgcacaagagagtcctagatgttttacctggggtttcaggaggcgtgactagggtgttagttggacagccattcgatacaattaaaactagattacaggtcatgggtcagggtacggcattggctaaaatgcttccaccttctgatgtgtatattaactcttccgactgtttgaaaaaaatggtacgtaacgaaggtgctttgtctctgtaccgtggtgtggttgcgcctcttttaggaaatatggtgctgcttggaattcatttccctaccttttcgaacacaagaaagtaccttgagtctgttgacgctacaccagcgggcgaatttccttactggaaggtgttagctgcaggtggcgcagcagggctcgcaggatctttcatatcttgtccttcggagcacatacgtactaagatgcaattacagcgtagagcggcccttgccgcacagatgggtttgaaggctcaagggctggagacttacaagggatcctgggattgtgctgtgcaaattttgaggaaccacggcataaagggactctacaggggaatgaccagtacggtgttaagggatatacaaggctacgcatggttcttcctctgttacgaagctacattgcacgccttagcaggaccagctcatacacgttctgaattggattacaagcacgtcttgggagctggagttatggcagggttcggtctctgggggtccatgtttccaatcgacacaataaaatccaaaatgcagggtgattctcttagtaacccccagtatcgtaacacgctggattgtctccgacagagtgtagcagttgaaggtttcgggggcctttttagagggttcggcgctgcaatgtacagagctattccagttaatgcaggtatatttcttgccgtagagggaacaagacaacttctaaacaagtacgagggttacattgatgagaagttgggtatttctgttcctgcttctgcggcgacagttcctgctccggcgcaatagggatccatttaaatgtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagactagtcgtcaaagggcgacaccccctaattagcccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgac

SEQ ID NO: 11 - DNA, 10881 bp, Artificial Sequence
  pMBXO86
tcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgaaaatgcgtacgggagttgcagtcgatctagcttccggaacagcggcaggggctgcccaattactcgtcggtcacccttttgacactatcaaagtgaatatgcaagttggatctgccgatactactgcaatgggagctgctagaaggattgtgggaactcacggccctttaggaatgtatagaggactggctgctcctttggcgacggtagcagcttttaacgcagtcctgttcagtagctggggtgctactgagagaatgttgtcccccgacggtggatgttgtcctcttacggtgggtcaagctatgctcgcgggaggtttagctggtgtcccagtaagcttgcttgccaccccaacagaactactcaaatgtcgtcttcaagctcaaggtggcgctagacctccgccaggtatggtttattcccttgctgatattcgtgcaggtcgtgctctttttaatggtcctctcgacgttttaagacatgtggttaggcatgaagggggttggttgggcgcctacagaggacttggtgcgacactcctccgtgaagttccgggcaacgcggcttacttcggtgtatatgagggttgtaaatatggtttagctaggtggcaatgtatccctacttccgagctcggcccggccagccttatgacagctggaggtgttggtggagccgctttttggatcgttacctaccctttcgatgttgtgaagtctagactccaaacccagaacatacacgcgcttgatcgatatcacggaacctgggattgtatgacacgtctctactctgcccagggttggcaggcactgtggagagggtttggcccttgcatggctagatctgtccccgccaatgcggttgcatttctggcgtttgaacaagtacgagctgctttgtcccattgaggatccatttaaatgtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagactagtcgtcaaagggcgacaccccctaattagcccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgac

SEQ ID NO: 12 - DNA, 11380 bp, Artificial Sequence
  pMBXO84
tcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcaccgaagaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggaacacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcttcgaaaaaatgtctagtgatgccatgaccatcaatgagtctcttatggaagtcgaacatactccagctgtgcataaaaggattcttgacattttaccgggtatcagtggcggggttgccagagttatgataggtcagcccttcgacacaatcaaagtgcgtctacaagtgttggggcagggtacggctctcgctgccaaacttcctcctagtgaagtttacaaggacagcatggattgcattcgtaagatgattaagtcggagggtccactaagcttttacaagggaacagttgccccactcgtcggaaacatggtattgcttggcatccattttccggtcttttccgcggttagaaagcagttggagggtgatgatcattactctaacttttcacacgccaatgtactgcttagcggcgctgcggcaggagctgcgggatcactcatttcggctcctgttgaactggttagaacgaaaatgcaaatgcaaaggcgagccgcacttgcgggtacagtggctgctggtgcagctgcatctgctggagctgaggagttctataagggaagtcttgattgtttcaaacaagttatgtctaagcatgggattaaaggattgtataggggttttacttcaactatactacgagatatgcagggttatgcttggttcttcctcggatatgaggcgactgtcaatcacttcttgcaaaatgcgggaccaggtgttcataccaaggctgacttgaattaccttcaagtgatggccgctggggttgttgctggatttggattatggggctccatgtttccaatcgataccatcaaatctaaactccaagccgatagctttgccaaacctcaatattcatccacaatggattgtcttaagaaagtattagcaagtgagggacaggccggcttgtggagagggttcagcgcagcaatgtatagagcaataccggtgaacgctggcattttcctcgctgttgaagggacacgtcagggtataaagtggtacgaggaaaacgtggaacacatctacggaggtgtcattggtcccgctacgcctactgcagcacaatgacgaaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgac

SEQ ID NO: 13 - DNA, 12156 bp, Artificial Sequence
  pMBXO71
tcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcaccgaagaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatgaactaaaggggatcgctctccctgagggttcagataaactcttttcagtcagtattgatggtacattgcgagtttgggactgcaattctggtcagtgtgtacattccatcaaccttgacgcagaagcagggtctctaatcagtgaaggcccttgggttttccttggcttgccaaacgctataaaggcttttaacgttcaaaccagtcaagatttgcatcttcaagcagcaggggtggttggtcaggtgaatgcaatgactattgcaaacggaatgctttttgctggaacaagttctggtagtatcttagtctggaaagctactacagactctgagtctgatccattcaaatacttgacatctcttgagggacatagtggtgaagtcacttgttttgctgttggaggtcaaatgctatactctggttctgtcgataaaacaatcaagatgtgggatctcaacaccctgcaatgtataatgaccctgaagcaacataccggcactgtcacttcactcttatgttgggataaatgtttgatatcgtcttccttggatgggaccataaaagtttgggcttattctgaaaacggaatcttgaaagttgttcaaactcgcagacaagaacagagtagtgttcatgctctttctggtatgcatgatgcagaagccaaaccgataatattctgctcttaccaaaacggaaccgttggcattttcgacctaccatcttttcaagaaagaggaaggatgttctctacgcacacgatcgccacactcacaattggtcctcaaggattgttattcagtggagacgagagtggtaacttgcgtgtatggaccttagctgctggcaacaaagtttagtcttttcgactaaagaattctgatttaattttgtggtttatatgttgagttaactgttaagagagttttattttgtaataggtgtatcagtcaataaacaatctttgtatcaaccaaatgtaatttttctcgttaattcgatttcagagtttttactttaagataaacaaactctttcacacatcatttaatgaaagtggagaagcttaaaaaacaaacaaagaaactgatccatttttggcgggtcttcttctactcttattcatatgtgttaacgaactatagcgtaaaattcagagcaagcgatctccgatttgaacgtggctatcaccggaggcccaccactacgggcgatacgctctaagtgaggattaaagtgctctggtggtgacgttgaagaaactcgcccatggtttttgttatctctgcagccaagtgtcgttctttcttcgccacttctcatcaagctacagtgaatttaaaaatggcgtctttctttgatctcgtatacataagctggattggtttcttaaacaaattcctctccttttgggtcttctgggtttgccttgtaagtgtttgtgtttttgcctctgagaaaaaatcttcgaaaaaatgtctagtgatgccatgaccatcaatgagtctcttatggaagtcgaacatactccagctgtgcataaaaggattcttgacattttaccgggtatcagtggcggggttgccagagttatgataggtcagcccttcgacacaatcaaagtgcgtctacaagtgttggggcagggtacggctctcgctgccaaacttcctcctagtgaagtttacaaggacagcatggattgcattcgtaagatgattaagtcggagggtccactaagcttttacaagggaacagttgccccactcgtcggaaacatggtattgcttggcatccattttccggtcttttccgcggttagaaagcagttggagggtgatgatcattactctaacttttcacacgccaatgtactgcttagcggcgctgcggcaggagctgcgggatcactcatttcggctcctgttgaactggttagaacgaaaatgcaaatgcaaaggcgagccgcacttgcgggtacagtggctgctggtgcagctgcatctgctggagctgaggagttctataagggaagtcttgattgtttcaaacaagttatgtctaagcatgggattaaaggattgtataggggttttacttcaactatactacgagatatgcagggttatgcttggttcttcctcggatatgaggcgactgtcaatcacttcttgcaaaatgcgggaccaggtgttcataccaaggctgacttgaattaccttcaagtgatggccgctggggttgttgctggatttggattatggggctccatgtttccaatcgataccatcaaatctaaactccaagccgatagctttgccaaacctcaatattcatccacaatggattgtcttaagaaagtattagcaagtgagggacaggccggcttgtggagagggttcagcgcagcaatgtatagagcaataccggtgaacgctggcattttcctcgctgttgaagggacacgtcagggtataaagtggtacgaggaaaacgtggaacacatctacggaggtgtcattggtcccgctacgcctactgcagcacaatgacgaaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgac

SEQ ID NO: 14 - DNA, 11801 bp, Artificial Sequence
  pMBXO107
tcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcaccgaagaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggaacacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcaacggttccggcggtatagagttgggtaattcgaaaccgcacagatccaattcgattagcagatatttggtgtctaaatgtttattttgtgatatgttcatgtttgaaatggtggtttcgaaaccagggacaacgttgggatctgatagggtgtcaaagagtattatggattgggacaatttcggtcatgagttgcaaattcaagtatatcgttcgattatgaaaattttcgaagaatatcccatttgagagagtctttacctcattaatgtttttagattatgaaattttatcatagttcatcgtagtctttttggtgtaaaggctgtaaaaagaaattgttcacttttgttttcgtttatgtgaaggctgtaaaagattgtaaaagactattttggtgttttggataaaatgatagtttttatagattcttttgcttttagaagaaatacatttgaaattttttccatgttgagtataaaataccgaaatcgattgaagatcatagaaatattttaactgaaaacaaatttataactgattcaattctctccatttttatacctatttaaccgtaatcgattctaatagatgatcgattttttatataatcctaattaaccaacggcatgtatggataattaaccgatcaactctcacccctaatagaatcagtattttccttcgacgttaattgatcctacactatgtaggtcatatccatcgttttaatttttggccaccattcaattctgtcttgcctttagggatgtgaatatgaacggccaaggtaagagaataaaaataatccaaattaaagcaagagaggccaagtaagataatccaaatgtacacttgtcattgccaaaattagtaaaatactcggcatattgtattcccacacattattaaaataccgtatatgtattggctgcatttgcatgaataatactacgtgtaagcccaaaagaacccacgtgtagcccatgcaaagttaacactcacgaccccattcctcagtctccactatataaacccaccatccccaatctcaccaaacccaccacacaactcacaactcactctcacaccttaaagaaccaatcaccaccaaaaattcgaaaaaatgtctagtgatgccatgaccatcaatgagtctcttatggaagtcgaacatactccagctgtgcataaaaggattcttgacattttaccgggtatcagtggcggggttgccagagttatgataggtcagcccttcgacacaatcaaagtgcgtctacaagtgttggggcagggtacggctctcgctgccaaacttcctcctagtgaagtttacaaggacagcatggattgcattcgtaagatgattaagtcggagggtccactaagcttttacaagggaacagttgccccactcgtcggaaacatggtattgcttggcatccattttccggtcttttccgcggttagaaagcagttggagggtgatgatcattactctaacttttcacacgccaatgtactgcttagcggcgctgcggcaggagctgcgggatcactcatttcggctcctgttgaactggttagaacgaaaatgcaaatgcaaaggcgagccgcacttgcgggtacagtggctgctggtgcagctgcatctgctggagctgaggagttctataagggaagtcttgattgtttcaaacaagttatgtctaagcatgggattaaaggattgtataggggttttacttcaactatactacgagatatgcagggttatgcttggttcttcctcggatatgaggcgactgtcaatcacttcttgcaaaatgcgggaccaggtgttcataccaaggctgacttgaattaccttcaagtgatggccgctggggttgttgctggatttggattatggggctccatgtttccaatcgataccatcaaatctaaactccaagccgatagctttgccaaacctcaatattcatccacaatggattgtcttaagaaagtattagcaagtgagggacaggccggcttgtggagagggttcagcgcagcaatgtatagagcaataccggtgaacgctggcattttcctcgctgttgaagggacacgtcagggtataaagtggtacgaggaaaacgtggaacacatctacggaggtgtcattggtcccgctacgcctactgcagcacaatgacgaaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgac

SEQ ID NO: 15 - DNA, 14758 bp, Artificial Sequence
  pMBXO75
gcgtataatggactattgtgtgctgataaggagaacataagcgcagaacaatatgtatctattccggtgttgtgttcctttgttattctgctattatgttctcttatagtgtgacgaaagcagcataattaatcgtcacttgttctttgattgtgttacgatatccagagacttagaaacgggggaaccgggatgagcaaggtaaaaatcggtgagttgatcaacacgcttgtgaatgaggtagaggcaattgatgcctcagaccgcccacaaggcgacaaaacgaagagaattaaagccgcagccgcacggtataagaacgcgttatttaatgataaaagaaagttccgtgggaaaggattgcagaaaagaataaccgcgaatacttttaacgcctatatgagcagggcaagaaagcggtttgatgataaattacatcatagctttgataaaaatattaataaattatcggaaaagtatcctctttacagcgaagaattatcttcatggctttctatgcctacggctaatattcgccagcacatgtcatcgttacaatctaaattgaaagaaataatgccgcttgccgaagagttatcaaatgtaagaataggctctaaaggcagtgatgcaaaaatagcaagactaataaaaaaatatccagattggagttttgctcttagtgatttaaacagtgatgattggaaggagcgccgtgactatctttataagttattccaacaaggctctgcgttgttagaagaactacaccagctcaaggtcaaccatgaggttctgtaccatctgcagctaagccctgcggagcgtacatctatacagcaacgatgggccgatgttctgcgcgagaagaagcgtaatgttgtggttattgactacccaacatacatgcagtctatctatgatattttgaataatcctgcgactttatttagtttaaacactcgttctggaatggcacctttggcctttgctctggctgcggtatcagggcgaagaatgattgagataatgtttcagggtgaatttgccgtttcaggaaagtatacggttaatttctcagggcaagctaaaaaacgctctgaagataaaagcgtaaccagaacgatttatactttatgcgaagcaaaattattcgttgaattattaacagaattgcgttcttgctctgctgcatctgatttcgatgaggttgttaaaggatatggaaaggatgatacaaggtctgagaacggcaggataaatgctattttagcaaaagcatttaacccttgggttaaatcatttttcggcgatgaccgtcgtgtttataaagatagccgcgctatttacgctcgcatcgcttatgagatgttcttccgcgtcgatccacggtggaaaaacgtcgacgaggatgtgttcttcatggagattctcggacacgacgatgagaacacccagctgcactataagcagttcaagctggccaacttctccagaacctggcgacctgaagttggggatgaaaacaccaggctggtggctctgcagaaactggacgatgaaatgccaggctttgccagaggtgacgctggcgtccgtctccatgaaaccgttaagcagctggtggagcaggacccatcagcaaaaataaccaacagcactctccgggcctttaaatttagcccgacgatgattagccggtacctggagtttgccgctgatgcattggggcagttcgttggcgagaacgggcagtggcagctgaagatagagacacctgcaatcgtcctgcctgatgaagaatccgttgagaccatcgacgaaccggatgatgagtcccaagacgacgagctggatgaagatgaaattgagctcgacgagggtggcggcgatgaaccaaccgaagaggaagggccagaagaacatcagccaactgctctaaaacccgtcttcaagcctgcaaaaaataacggggacggaacgtacaagatagagtttgaatacgatggaaagcattatgcctggtccggccccgccgatagccctatggccgcaatgcgatccgcatgggaaacgtactacagctaaaagaaaagccaccggtgttaatcggtggcttttttattgaggcctgtccctacccatcccctgcaagggacggaaggattaggcggaaactgcagctgcaactacggacatcgccgtcccgactgcagggacttccccgcgtaaagcggggcttaaattcgggctggccaaccctatttttctgcaatcgctggcgatgttagtttcgtggatagcgtttccagcttttcaatggccagctcaaaatgtgctggcagcaccttctccagttccgtatcaatatcggtgatcggcagctctccacaagacatactccggcgaccgccacgaactacatcgcgcagcagctcccgttcgtagacacgcatgttgcccagagccgtttctgcagccgttaatatccggcgcagctcggcgatgattgccgggagatcatccacggttattgggttcggtgatgggttcctgcaggcgcggcggagagccatccagacgccgctaacccatgcgttacggtactgaaaactttgtgctatgtcgtttatcaggccccgaagttcttctttctgccgccagtccagtggttcaccggcgttcttaggctcaggctcgacaaaagcatactcgccgtttttccggatagctggcagaacctcgttcgtcacccacttgcggaaccgccaggctgtcgtcccctgtttcaccgcgtcgcggcagcggaggattatggtgtagagaccagattccgataccacatttacttccctggccatccgatcaagtttttgtgcctcggttaaaccgagggtcaatttttcatcatgatccagcttacgcaatgcatcagaagggttggctatattcaatgcagcacagatatccagcgccacaaaccacgggtcaccaccgacaagaaccacccgtatagggtggctttcctgaaatgaaaagacggagagagccttcattgcgcctccccggatttcagctgctcagaaagggacagggagcagccgcgagcttcctgcgtgagttcgcgcgcgacctgcagaagttccgcagcttcctgcaaatacagcgtggcctcataactggagatagtgcggtgagcagagcccacaagcgcttcaacctgcagcaggcgttcctcaatcgtctccagcaggccctgggcgtttaactgaatctggttcatgcgatcacctcgctgaccgggatacgggctgacagaacgaggacaaaacggctggcgaactggcgacgagcttctcgctcggatgatgcaatggtggaaaggcggtggatatgggattttttgtccgtgcggacgacagctgcaaatttgaatttgaacatggtatgcattcctatcttgtatagggtgctaccaccagagttgagaatctctataggggtggtagcccagacagggttctcaacaccggtacaagaagaaaccggcccaaccgaagttggccccatctgagccaccataattcaggtatgcgcagatttaacacacaaaaaaacacgctggcgcgtgttgtgcgcttcttgtcattcggggttgagaggcccggctgcagattttgctgcagcggggtaactctaccgccaaagcagaacgcacgtcaataatttaggtggatattttaccccgtgaccagtcacgtgcacaggtgtttttatagtttgctttactgactgatcagaacctgatcagttattggagtccggtaatcttattgatgaccgcagccaccttagatgttgtctcaaaccccatacggccacgaatgagccactggaacggaatagtcagcaggtacagcggaacgaaccacaaacggttcagacgctgccagaacgtcgcatcacgacgttccatccattcggtattgtcgacgacctggtaagcgtattgtcctggcgtttttgctgcttccgagtagcaatcctcttcaccacaaagaaagttacttatctgcttccagttttcgaacccttcttctttgagccgcttttccagctcattcctccacaaaacaggcacccatcctctgcgataaatcatgattatttgtcctttaaataaggctgtagaactgcaaaatcgctctcgttcacatgctgtacgtagatgcgtagcaaattgccgttccatccctgtaatccaccttctttggaaagatcgtccttgacctcacgaagaaccttatccaatagccctgcggcacaagaaattgcctgctctggatcagcaaattcatattgattaataggtgattgccacacaccaaaaacaggaatcatcttttcggctaaacgcctctcctgttctttcttaatctcaagttgtaagcggaccagctcaccatccatcattttttgtagatcatgcgccactattcacccccactggccatcagcaaataaagcttcatactcggacaccggcaggcggcttccacggattgaaaggtcaagccaaccacgtccagatgggtcagccttatccgattcttcccaccgttctgcagctgtagcaaccaggcattctaccgccttcatgtagtcttctgtacggaaccagccgtagttaatgccaccatcagtaactgcccaggccatctttttctcttcggcctcaatagcccggatgcggttatcgcacagctcgcgacagtacttcagctgttcgtaatccagttgcttcaggaactctggtgtcgacgtcatagtggcttcaccttataggcttttagaagcgccctggcttcgtctgtgtggtcttccatgctcttatcgctggcaatgcagcaataaactccctcactatctgagaacccgttcatccgaatgatcgtgaatggaagttcccggccagttttataatcgctatagcttgtcgcgtcgtggctgaccttgaccacataagggtcgtagccctccacgatgacaaggcattcccgttgttttcccattacccctccggttatatcgccacggcttgccgctggcttagaaacgctttcagcagccttatttcgcgtactgatagcaggtccataaattcggtcatgtacagcgaggcgaacgttctcgcgatgctggccactggccacaggcgtaccgcctccatttcggttgctggcaacgcgttctccgcccacgcctccggtaccgccaccgggatagcctccagtgcctggataattactgattgtggggcgtccggaacgtgctctgttttggatcgagggttaccatgtatatctatatttagatccaaatcgcgatccacttcgatggtggttttttccaccttacgtgcgtgaattgataaaccggcctcgcggcgcttctccacgatattcatgaggaactcgaccgagtccgggtcaatggaacgcatcgtggggcgtgcatcgccgtctctggcgcgtctggtcttactggatagccccatagactccaggatgcctatgcagaggtctgcaggcgctttcttcttgcctttctctgtgttgaagccgccgatgcgtaaaacgttgtttagcagatcgcgccgttccggcgtgagcaggttatctctggcgcgtttgagggcgtccatgtctgcttcaccttccagggtttttggatcgataccgcagtcgcggaagtactgctgcagcgtcgccgatttgagggtgtagaaaccacgcatgcctatctcaacagcaggggtcgatttcactcggtaatcggttatggccgggaatttagcctggaactctgcgtcggcctgttcccgcgtcatggccgtagtgacgaactgctgccatcttccggcaacgcgataagcgtaggtaaagtgaatcaacgcttcttcacggtcaaggcgacgggcggttatctcatccagctgcatggtttcaaacaggcgcacttttttcaggccgccgtcgaaatagaattttaacgccacctcgtcgacatccagctgcagctccttttcgatgtcccagcggaccagctgggcctgctcatccagggacagggtgcgtttttttatcaactcatcgtgttcggcctggtcaggagtatcgacactcaggtggcgctccataagctgctcaaagaccagttcacgggcttctttacgtaaatccttaccgatgctgtttgcaagcgcgtcggtggccataggcgcgacctgatagccatcatcatgcatgatgcaaatcatgttgctggcataatcatttctggccgatgcctcgagcgcggcggctttaattttgagctgcatgaatgaagagttagccacgccgagtgaaattcggtcaccgtcaaagacaacgtctgtcagcagcccggagtggccagccgtttcgagcaaggcctgcgcgtaggcgcgtttgattttttccggatcggtttcacgtttaccgcgaagcttgtcgaaaccgataatgtattcctgagctgtacggtcgcggcgcagcatctggatggcgtcgctggggaccacttcgccgcagaacatgccgaaatggcggtggaagtgtttctcctcaatcgatacacctgaagatatcgacgggctgtagatgaggccgtcatattttttcaccatcactttaggctggttggtgaaatcgtcgacttccttctcctgtttgtttttctggttaacgcagagaaactttttgtcagggaactgtagtctcagctgcatggtaacgtcttcggcgaacgtcgaactgtcggtggccagcatgattcgttcgccgcgttgcactgcagcgataacctcggtcatgatccgatttttctcggtataaaatacgcggataggcttgttggtttcgcggttgcgaacgtcgaccgggagttcaatcacgtgaatttgcagccaggcaggtaggcccagctcctcgcgtcgcttcatcgccagttcagccaggtcaacaagcagatcgttggcatcggcatccaccataatggcatgctcttcagtacgcgccagcgcgtcgataagcgtgttgaatacgcctaccgggttttccatcgcacgcccggccagaatggcacgcaggccctgtgttgcttcatcgaagccgaagaagtcatgctggcgcatcagcggttgccagcagcctttaagtatggagttgatgcaaatagtcagcttgttggcatatggcgccatttcctgatagccgggatcctgataatgcagaatgtcggctttcgcgcctttcccttcggtcatcatttcatgcaggccgcctatcagggatacgcggtgcgcgacggaaacgccacgcgtggactgcagcatcagtggacgcaggaggcctgtcgatttacccgaccccatcccggcgcggacaataacgatgccctgcagctgtgcggcgtatgtcatcacctcatcggtcatcctggaggtttcaaaccgtttgtaagtgatgtgtgacgggcgaaggttcgggttggtgatgcgttcactgaacgaacgtgatgtttgcgcggcacggcatttgcgattcaaccggcgcgtaatgtgatctttaacggtaccgttataaatttctgcgatacccatatcccgcagcgtgctgctgaaaaggcgcataagttctttcgggctgtttggtaccgggcatgtcagcatgccaatatcaacggcgcgaagcagttctttggcaaaagtgcgtctgttcagacgcgggagagtacgcagcttattcagcgtgatcgacaacagatcggttgcacggctcagatgatttctcgttaactggcgagcgacttccttcagccctctcaggctgtgcaggtcgttaaaatcgctgcattccagctcagggtcatcctcaaaagttgggtaaacacatttgacgccggaaaacttctccatgatgtcgaatccggtgcggaggcctgtgttgccttttccttcagctgaggatttgcggtcgttatcgagagcgcaagtgatttgcgcagccgggtacatgttcaccagctgctcgacaacgtgaatcatgttgttagcggaaaccgcaatgactaccgcgtcaaagcgttttttcgggtcgtttctggtcgccagccagatggatgccccggtggcgaaaccctctgcagtcgcaattttttgcgccccctgcaggtcgccaataacaaagcatgcaccgacgaaatcaccgttagtgatggcgctggtctggaacttgccaccattcagatcgatacgttgccagccaacaatccgcccgtcttttcttccgtccaggtgggacagaggtatcgccatgtaagttgttggtccacggctccatttcgcactgtcgtgactggtcacgcgacgtatatcacaagcgccaaatacgtcacgaattcccttttttaccgcataaggccaggagccatcttcagctggcgaatgttcccaggcgcgatggaaagccaaccatccaagcaggcgttcctgctccatctgattgttttttaaatcattaacgcgttgttgttcagctcggaggcggcgtgcttcagcctggcgctccatgcgtgcacgttcttcttccggctgagcgaccacggtcgcaccattccgttgctgttcacggcgatactccgaaaacaggaatgaaaagccactccaggagccagcgtcatgcgctttttcaacgaagttaacgaaaggataactgatgccatccttgctctgctcaaggcgtgaatagatttccacacggcctttaaggctcttctgcagagcttccggggaggaattattgtaggtggtatagcgctctacaccaccgcgcggattgagctgaatcttatcagcacacgcaggccagttgataccggccatcttcgccagctcagtcagctcatcacgtgccgcgtcaagcagtgaaaacggatcgctgccaaagcgctccgcgtagaattcttgtaaggtcattttttagcctttccatgcgaattagcattttttcgggttgaaaaaatccgcaggagcagccacaataaacgcactatctttctgaaggacgtatctgcgttatcgtggctacttcctgaaaaaggcccgagtttgccgactcgggttttttttcgtcttttttcggctgctacggtctggttcaaccccgacaaagtatagatcggattaaaccagaattatagtcagcaataaaccctgttattgtatcatctaccctcaaccatgaacgatttgatcgtaccgactacttggtgcacaaattgaagatcacttttatcatggataacccgttgagagttagcactatcaaggtagtaatgctgctcgtcataacgggctaatcgttgaattgtgatctcgccgttattatcacaaaccagtacatcctcacccggtacaagcgtaagtgaagaatcgaccaggataacgtctcccggctggtagtttcgctgaatctggttcccgaccgtcagtgcgtaaacggtgttccgttgactcacgaacggcaggaatcgctctgtgttggcaggttctccaggctgccagtctctatccggtccggtctctgtcgtaccaataacaggaacgcggtctggatcagattcagtgccatacagtatccattgcacgggcttacgcaggcattttgccagcgatagcccgatctccagcgacggcatcacgtcgccacgttctaagttttggacgcccggaagagagattcctacagcttctgccacttgcttcagcgtcagtttcagctctaaacggcgtgctttcagtcgttcgcctcgtgttttcatacccttaatcataaatgatctctttatagctggctataatttttataaattatacctagctttaattttcacttattgattataataatccccatgaaacccgaagaacttgtgcgccatttcggcgatgtggaaaaagcagcggttggcgtgggcgtgacacccggcgcagtctatcaatggctgcaagctggggagattccacctctacgacaaagcgatatagaggtccgtaccgcgtacaaattaaagagtgatttcacctctcagcgcatgggtaaggaagggcataacaggggatcctctagacgcagaaaggcccacccgaaggtgagccagtgtgattacatttgcggcctaactgtggccagtccagttacgctggagtcactagtgcggccgcgacaacttgtctagggcccaatggcccgggactggcgcgccgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcggtaccaaaatgtcctcagatgcaatgactatcaatgagagtctgatggaagtcgagcacacacctgccgtacataaaagaatccttgatattctccccggaatttctggcggagtagctagagttatgattggacagccattcgatacaattaaagttaggctccaagtgcttggtcaggggactgcacttgccgctaagcttcctccttctgaggtgtataaggattcaatggattgcattcgcaaaatgattaagtccgaaggaccactttcattttacaagggaacagttgccccccttgttggaaatatggttcttcttggtatccacttcccagttttttctgccgttaggaaacaacttgagggagatgaccattactctaatttcagtcatgcaaatgttcttctcagtggggccgcagctggcgctgcaggtagtttgattagtgcacctgtggagttggtcaggacaaagatgcaaatgcaacgcagagcagctttggccgggactgtggcagccggtgccgctgcttctgcaggagccgaagagttttataagggctcccttgactgcttcaaacaagtaatgtccaaacatggtattaagggtctttaccgtggttttacatccactatcctccgcgatatgcagggttacgcttggttttttcttggctacgaggcaactgtcaatcattttcttcagaatgctggccctggagttcataccaaagcagatcttaactaccttcaagtcatggcagctggagttgttgcaggctttggattgtggggatctatgtttcctatagatacaatcaagagtaagctccaagccgatagcttcgcaaagccacagtacagtagtaccatggactgcctgaaaaaggttttggcaagcgaaggtcaagcagggttgtggaggggcttctctgctgcaatgtatcgtgccatacctgtgaacgctggtatatttctcgcagttgaaggaactagacagggaatcaagtggtacgaagaaaacgtcgaacacatttatggcggtgttattggacctgctactcctaccgctgcacagtaatctagagcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaaggcgcgccagtcccgggccattagacttgaagtcaagcggccgcttacaactggaccttgctggtacatagaactgattaactgaccatttaaatcataccaacatggtcaaataaaacgaaaggctcagtcgaaagactgggcctttcgttttaatctgatcggcacgtaagaggttccaactttcaccataatgaaataagatcactaccgggcgtatttttgagttatcgagattttcaggagctaaggaagctaaaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaacctaggtgacagaagtcaaaagcctccggtcggaggcttttgactttctgctagatctgtttcaatgcggtgaagggccaggcagctggggattatgtcgagacccggccagcatgttggttttatcgcatattcagcgttgtcgcgtttacccaggtaaaatggaagcagtgtatcgtctgcgtgaatgtgcaaatcaggaacgtaaccgtggtacatagatgcagtcccttgcgggtcgttcccttcaacgagtatgacgcggtgcccttgcaaggctaaccattgcgcctggtgtactgcagatgaggttttataaacccctcccttgtgtgacataacggaaagtacaaccgggtttttatcgtcaggtctttggtttgggttaccaaacacactccgcatatggctaatttggtcaattgtgtagccagcgcgacgttctactcggcccctcatctcaaaatcaggagccggtagacgaccagctttttccgcgtctctgatagcctgcggtgttacgccgatcaggtctgcaacttctgttataccccagcggcgagtaatacgacgcgcttccgggctgtcatcgccgaactgtgcgatggcaatagcgcgcgtcatttcctgaccgcgattgatacagtctttcagcaaattaattaacgacatcctgtttcctctcaaacatgcccttatctttgtgtttttcatcatactttacgtttttaaagcaaagcaacataaaaaaagcaaagtgacttagaaaacgcaaagttaaggttcaaatcaattttttgatgcgctacagaagctatttagcttcatctaagcgcaacggtattacttacgttggtatatttaaaacctaacttaatgattttaaatgataataaatcataccaattgctatcaaaagttaagcgaacatgctgattttcacgctgtttatacactttgaggcatctctatctcttccgtctctatattgaaacacaatcaaagaacatcaatccatgtgacatcccccactatctaagaacaccataacagaacacaacataggaatgcaacattaatgtatcaataattcggaacatatgcactatatcatatctcaattacggaacatatcagcacacaattgcccattatacgc

SEQ ID NO: 16 - DNA, 13245 bp, Artificial Sequence
  pMBXS1089
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagttatcatcatcatcatagacacacgaaataaagtaatcagattatcagttaaagctatgtaatatttacaccataaccaatcaattaaaaaatagatcagtttaaagaaagatcaaagctcaaaaaaataaaaagagaaaagggtcctaaccaagaaaatgaaggagaaaaactagaaatttacctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatggtagaggagctcgccgcttggtatctgcattacaatgaaatgagcaaagactatgtgagtaacactggtcaacactagggagaaggcatcgagcaagatacgtatgtaaagagaagcaatatagtgtcagttggtagatactagataccatcaggaggtaaggagagcaacaaaaaggaaactctttatttttaaattttgttacaacaaacaagcagatcaatgcatcaaaatactgtcagtacttatttcttcagacaacaatatttaaaacaagtgcatctgatcttgacttatggtcacaataaaggagcagagataaacatcaaaatttcgtcatttatatttattccttcaggcgttaacaatttaacagcacacaaacaaaaacagaataggaatatctaattttggcaaataataagctctgcagacgaacaaattattatagtatcgcctataatatgaatccctatactattgacccatgtagtatgaagcctgtgcctaaattaacagcaaacttctgaatccaagtgccctataacaccaacatgtgcttaaataaataccgctaagcaccaaattacacatttctcgtattgctgtgtaggttctatcttcgtttcgtactaccatgtccctatattttgctgctacaaaggacggcaagtaatcagcacaggcagaacacgatttcagagtgtaattctagatccagctaaaccactctcagcaatcaccacacaagagagcattcagagaaacgtggcagtaacaaaggcagagggcggagtgagcgcgtaccgaagacggtagatctctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtaccaaggtttcatgcgtatcgtgacagatgttacataatgacaaattccccagctggagcacctttatccctgctgtttgcatgaaattagcttgtcttgtagttccctccagcaaaaagaagtctgaaacaaaacaacatttcgaaaaaaaggcatccatgagttagcatttctacagttgtctatagaggggaaggctgcacgacaaagtttccaggcttggaaacaacctcttatgtaaaatttttcgtatgtatcagatgatttgtttgcgttacggcatctccacctaacatcaccttcatcatgcgcctatggtctttctcttgcctgttttatacgtaaaattggaaacgacagaaacttttgccatctttattaaaggaaggcaaatatgcaaatataggcatcaagatcacagttagtggattatcatctttgtaggttaacatgtcctaccccaggggagcttatactcaagtactccatgcattttcatgaaatgagaaaaaacgatttttaagagaaatgtactttcttgtatttatgccaaatggcaaggactgaaagggaaaaactaagaaagggaacgttacagtaaggctctgtggggactggggacttcagagaaacgtgaaccctgcttccttcctctgcatgaacataacaccagaggtttccagcctttcacacagttgttgatggcttcacacaattcatctctacctcctgactctttataaggacccccagcatcaccacaattgcacaagtacaggcattagatccacaagaacacttgggcaggcaagcacctctttgatctttaagccgttgttatgttctatttctgagcatatggtttctagttatattctttttcttcattcgtttcatatctttgaagtgttgatgcaaatgcggtgaacaactatcaactgtgtactctccaagtgaatgcgaataatcatttcctgtgagaattgtgggctagataaacgaatgaaatgctgttttatctatgtcatgtgtggaaatttagttaattttccggtctttttatgcattgagatgggtatgctgtttttttagttgggtcccatcatcttgagaattctttcaaatttccttttctttatcctatataaaggatagagaaggcgtatgcctaggtgcaccaaccctgaaagttttattctaattgcgggaatggtttgtaatttttgcttgttcaggttctttttcgtggcctttcttttttttccccttattttgcttagtctttcacagtccaatttttgggaagtagtatatcttagtttggtcctaaggcaccatgttgtactgcaggaaaaaaaagagtaattgtattctgttttttccttgattactatatccctgttttaattaattttgtgcctttgttgtttgatgttggaacttcaatgcccataattagtcatttgacttgttttgggttttgacgctatcttgagtgccataggaaactggtagaatttagtaataattttatatagactgaatgttgagcccaccacaaatggtttccttctgtacaagtatttaataactcaagcacaggaaacatcagatctctaatctaaaggttaacaatgggctcaagcaggagcagtagttcagctctatctgtatatttagaagggctggatctacctgtccaccagcttttaattttaccctggcagctggataacttcttgtctgttaatttcatttagtgctgtgttattttcttcttgttgttcaggatggatgcttttgaatttctggaatttcgtattttgttctatctctttatgaaatgacgttatggcacactttttctgcatattcttgatgaaaataattacctagtcatttttttagttgcaggtttgtctgggactttgagtacccatgcaattcggtaccatgtcctctgatgcaatgacgattaacgaaagcctgatggaggttgagcacaccccagctgttcacaagaggatcctggatatactgccgggtattagcggcggtgtcgcgcgtgtcatgattgggcagccattcgacacgatcaaagtccgcctccaagtattgggccagggaaccgctctggccgcaaaattacctccgagtgaagtgtacaaggactctatggactgtatcagaaaaatgatcaagtccgagggtcctctttcattctataagggcacagtggctccactcgtgggcaacatggtgctactggggatccattttcccgtgttttccgccgtgcggaagcagctcgagggggatgatcattattcaaacttctcgcacgccaacgtgctcttgtccggcgctgctgccggagcggcgggcagcctgatctcggcaccggttgaactcgtccgcacaaagatgcagatgcagaggagggcagcattggccggcactgtcgccgccggcgcggccgcctcggcgggagcggaggagttctacaagggctctctcgactgcttcaaacaagtcatgagcaaacatggcattaagggcctatacaggggtttcaccagcactatccttcgggacatgcagggttacgcttggttcttcctcggttacgaggccactgtcaatcactttctccaaaatgccggaccaggcgttcatacaaaggcggatttgaactaccttcaagttatggcggctggcgtagttgccgggttcggactatgggggtcaatgtttcctatcgataccattaagtctaaacttcaagctgacagtttcgcgaagccccagtactcatccacgatggactgcctgaagaaggtcctggcttccgagggccaagccggtctttggagagggttcagcgcagcgatgtatcgagctatacctgtaaacgcaggaattttcctggcggtggagggcacccgccagggcataaagtggtacgaagagaatgtcgaacacatctacgggggcgtgatcgggcccgccacgccgacagcagcccaggaacagaagttaatctcggaggaggacctctgacccgggggcgcgccatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcaaacatgaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 17 - DNA, 13646 bp, Artificial Sequence
  pMBXS1090
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagttatcatcatcatcatagacacacgaaataaagtaatcagattatcagttaaagctatgtaatatttacaccataaccaatcaattaaaaaatagatcagtttaaagaaagatcaaagctcaaaaaaataaaaagagaaaagggtcctaaccaagaaaatgaaggagaaaaactagaaatttacctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatggtagaggagctcgccgcttggtatctgcattacaatgaaatgagcaaagactatgtgagtaacactggtcaacactagggagaaggcatcgagcaagatacgtatgtaaagagaagcaatatagtgtcagttggtagatactagataccatcaggaggtaaggagagcaacaaaaaggaaactctttatttttaaattttgttacaacaaacaagcagatcaatgcatcaaaatactgtcagtacttatttcttcagacaacaatatttaaaacaagtgcatctgatcttgacttatggtcacaataaaggagcagagataaacatcaaaatttcgtcatttatatttattccttcaggcgttaacaatttaacagcacacaaacaaaaacagaataggaatatctaattttggcaaataataagctctgcagacgaacaaattattatagtatcgcctataatatgaatccctatactattgacccatgtagtatgaagcctgtgcctaaattaacagcaaacttctgaatccaagtgccctataacaccaacatgtgcttaaataaataccgctaagcaccaaattacacatttctcgtattgctgtgtaggttctatcttcgtttcgtactaccatgtccctatattttgctgctacaaaggacggcaagtaatcagcacaggcagaacacgatttcagagtgtaattctagatccagctaaaccactctcagcaatcaccacacaagagagcattcagagaaacgtggcagtaacaaaggcagagggcggagtgagcgcgtaccgaagacggtagatctctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccctcggtggatgcttctttaataaagtaagtgttcaagatttatttttggtatttaatttacttgcttaagtcagatatattcccatcgttgcaggtttgtcacttagtattattattaagcgctctagcactaggactctggataaataagaaagtttattcacgaggctagagtagtaatcaataacataagcgtggtgtctaggtcagcggttatcttcatatgtagtgtgctccatggaaagtgaggtaggaggaaggtggtgacagtcccgtccgtcctttgtatccctccatgttcgggtatatcatagagctacaggctagacttagcttggcagactaggggagagccggtgctcgaagcaatccatgaggctttacatttaacataagttagtaaattaacccataggaatcatctctagactgaacctaccagtagttgtgcttggatataattatattcctacatatacatacacgttccctgcgattagatacccttggaatactctaaggtgaagtgctacagcggtatccgtgcgcttgcggatttatctgtgaccgtatcaaataccaacaggtagatacaaggaatcatctctcctatccattggtttatcatcttttaaaattatctcttgctctcctattgcctctgcaactgcggataggtgtttctcaacaatgaaggttgtgaagaatgctttgtgcaacaagatggatgacaagtatctcagccatagcctcatttgctttgtagaaaaggatatgtcggacacaatcactaagtatcaccgtggaaaggatgcactgtatgccctatctatatttaccatttagtaatatttatatggcttgtgctaactttatgttgtctttacaggcaataacattatttggaaggcatatctatatattactatttaagataatgtaatatctcaaagtttttataagctgcaatgaggtgagtttcacttagctttctaacttgttatgagttatagatgcatgccaccagtcattttttatcttgcatcagcccctgcctgttagaatatgtttctttgtctgggagtccatgtcaactagccaatttccaaatatatgaacaaaactatgtggcctttgtaacccaaatgagataaagactactctccatagaaatttagcaaacatggcactcaaagaaaatgtgttggatagtttcatcatgcatacaaaagcaacacttttgaactaccattccaaatcctttttgtaaattatctttgcttaacactacccctttgagcaaatgtggctttgtgcggaaaaaactcaaacttggtagggtagacatccatttatataattggatccatgtacataagttgttgagtacttcaagtacttacccttgtgatatacatctcaaatatattgaagaagagaagttctttttttgagagaggttgaagaagagaagtttgtccatagctgaagaggagttttatagtgtctagcttaccttgctgctgattgcatgtctaaaatgtcgtttaatttgggctataatgaaatattcaccaatatttctgctggtctattaaagtttaatagttactcgtaactcatttattttgggctataatttaatattcacctatgtttttgttagtctattttatttccctagtgtgcactagcttaaccccaaattagttttgaacacttaacctaaatgtgtctattatggtcagacactctctcacggcactctaacaaaaagtgaattttgttgttatgtttttgtcatgatctcacaagcaatgtacatgtacgtttctagagtgcaatcttatgctagcctgattgtgaatttagtgtagtttgttttctctttttgtagctacactaccaataacctattgtcctctagtcataccacgtaatcacaaggcaaatccctaactctcacctttaaaagcatgtctttattttcttgggtggcactaatacaaaatctttttcagcattcctatgtgcgatagcaagaaaacatggcataactcttgcttcactctaacaaaaaaaacacttttccaactttaaaacaatggtatctatgtgtttaatgatcaatcaagcatataatgacttacaagtttttacctatgccctttttgcatcatcttgtttgcaacagacaaactagatattcctttaggctataaacacatcagcatgataaagagattaggtaagtttgttatccctttttgcatatattctcgtctactccgtgtatataagcccctctcctccaactcgtccatccatcaccaagagcagtgggaggtaccatgtcctctgatgcaatgacgattaacgaaagcctgatggaggttgagcacaccccagctgttcacaagaggatcctggatatactgccgggtattagcggcggtgtcgcgcgtgtcatgattgggcagccattcgacacgatcaaagtccgcctccaagtattgggccagggaaccgctctggccgcaaaattacctccgagtgaagtgtacaaggactctatggactgtatcagaaaaatgatcaagtccgagggtcctctttcattctataagggcacagtggctccactcgtgggcaacatggtgctactggggatccattttcccgtgttttccgccgtgcggaagcagctcgagggggatgatcattattcaaacttctcgcacgccaacgtgctcttgtccggcgctgctgccggagcggcgggcagcctgatctcggcaccggttgaactcgtccgcacaaagatgcagatgcagaggagggcagcattggccggcactgtcgccgccggcgcggccgcctcggcgggagcggaggagttctacaagggctctctcgactgcttcaaacaagtcatgagcaaacatggcattaagggcctatacaggggtttcaccagcactatccttcgggacatgcagggttacgcttggttcttcctcggttacgaggccactgtcaatcactttctccaaaatgccggaccaggcgttcatacaaaggcggatttgaactaccttcaagttatggcggctggcgtagttgccgggttcggactatgggggtcaatgtttcctatcgataccattaagtctaaacttcaagctgacagtttcgcgaagccccagtactcatccacgatggactgcctgaagaaggtcctggcttccgagggccaagccggtctttggagagggttcagcgcagcgatgtatcgagctatacctgtaaacgcaggaattttcctggcggtggagggcacccgccagggcataaagtggtacgaagagaatgtcgaacacatctacgggggcgtgatcgggcccgccacgccgacagcagcccaggaacagaagttaatctcggaggaggacctctgacccgggggcgcgccatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcaaacatgaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 18 - DNA, 12494 bp, Artificial Sequence
  pMBXS1091
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagttatcatcatcatcatagacacacgaaataaagtaatcagattatcagttaaagctatgtaatatttacaccataaccaatcaattaaaaaatagatcagtttaaagaaagatcaaagctcaaaaaaataaaaagagaaaagggtcctaaccaagaaaatgaaggagaaaaactagaaatttacctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatggtagaggagctcgccgcttggtatctgcattacaatgaaatgagcaaagactatgtgagtaacactggtcaacactagggagaaggcatcgagcaagatacgtatgtaaagagaagcaatatagtgtcagttggtagatactagataccatcaggaggtaaggagagcaacaaaaaggaaactctttatttttaaattttgttacaacaaacaagcagatcaatgcatcaaaatactgtcagtacttatttcttcagacaacaatatttaaaacaagtgcatctgatcttgacttatggtcacaataaaggagcagagataaacatcaaaatttcgtcatttatatttattccttcaggcgttaacaatttaacagcacacaaacaaaaacagaataggaatatctaattttggcaaataataagctctgcagacgaacaaattattatagtatcgcctataatatgaatccctatactattgacccatgtagtatgaagcctgtgcctaaattaacagcaaacttctgaatccaagtgccctataacaccaacatgtgcttaaataaataccgctaagcaccaaattacacatttctcgtattgctgtgtaggttctatcttcgtttcgtactaccatgtccctatattttgctgctacaaaggacggcaagtaatcagcacaggcagaacacgatttcagagtgtaattctagatccagctaaaccactctcagcaatcaccacacaagagagcattcagagaaacgtggcagtaacaaaggcagagggcggagtgagcgcgtaccgaagacggtagatctctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtaccttgcatgccgtcgtcttaagcgtccgcgtgtgaaaatcggattttcgcatacggttgaaccggtcgcatgcaaagatcgcgatcttcgcagacgatttggcacatgcggttgcaccaaccgtatgcgaaaacccttctcgcccgtatgcaaaaaccatctttgttgtagtgtacggttcacaatggtttggatgggaaatcattgtgaaccaaaagtgatagactgatttcgacgagtgtttttttttaagtagtgccacaattttggtcatcatacgtcgtgtctaaaattgtaacttttgaaaaccaatttacattaaattaaatttataagactaaataaagacgatggtcattgaacaattgttgagaaaaatctacacacatgtgtgtccaacacaaatgtttacacatatactactatgttcatagtcgaagttagattttttttttccttaaagggaaagtctgttttcaaattttagacctcactccttccgtttcaaatatatcgtgtatttttttttctagggcaagcttttgaccaatgattactctattatgacacaatgttaaagggatagattcatattcaaaattactattataattataattttgtcatataaataatattttaagcaattgttagccaaaatctcgtcctaacgaaacaaaatacgccttatttttaaaaacacggagtatatccttaaatatttctctatccaatataaaaggtcaatcttttaaaattccgatcatcaataatttctcaaataattactttgaaataaaaaaacatatgcaaatttgtgtcgtcataatatccaatgaacttattcaaatttataaacttattttaattcaaaatttgatcattaattttttttttaaaaaaaaaccaaatcttatcataaacgtcaaatatatttttgatagtgggggcgataataccataaaactaacaacagaagagacatgatactactactgtaatcctaatacgtacgtacgtatacttctacgccggatgcataacttcagccttgtgagacacaacagttgctgcctagctcgtggtcgttggttttttcgctcgagaaaccactacgcgtaaaccgtgaagtatattatatatagccaactggtcttctcgcaaatccgcacatccctttctgcccctcgtcttctggtaccatgtcctctgatgcaatgacgattaacgaaagcctgatggaggttgagcacaccccagctgttcacaagaggatcctggatatactgccgggtattagcggcggtgtcgcgcgtgtcatgattgggcagccattcgacacgatcaaagtccgcctccaagtattgggccagggaaccgctctggccgcaaaattacctccgagtgaagtgtacaaggactctatggactgtatcagaaaaatgatcaagtccgagggtcctctttcattctataagggcacagtggctccactcgtgggcaacatggtgctactggggatccattttcccgtgttttccgccgtgcggaagcagctcgagggggatgatcattattcaaacttctcgcacgccaacgtgctcttgtccggcgctgctgccggagcggcgggcagcctgatctcggcaccggttgaactcgtccgcacaaagatgcagatgcagaggagggcagcattggccggcactgtcgccgccggcgcggccgcctcggcgggagcggaggagttctacaagggctctctcgactgcttcaaacaagtcatgagcaaacatggcattaagggcctatacaggggtttcaccagcactatccttcgggacatgcagggttacgcttggttcttcctcggttacgaggccactgtcaatcactttctccaaaatgccggaccaggcgttcatacaaaggcggatttgaactaccttcaagttatggcggctggcgtagttgccgggttcggactatgggggtcaatgtttcctatcgataccattaagtctaaacttcaagctgacagtttcgcgaagccccagtactcatccacgatggactgcctgaagaaggtcctggcttccgagggccaagccggtctttggagagggttcagcgcagcgatgtatcgagctatacctgtaaacgcaggaattttcctggcggtggagggcacccgccagggcataaagtggtacgaagagaatgtcgaacacatctacgggggcgtgatcgggcccgccacgccgacagcagcccaggaacagaagttaatctcggaggaggacctctgacccgggggcgcgccatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcaaacatgaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 19 - protein, 356 aa, Gonium pectorale
MVSMTMNDTLNQVEHTPVNPPHKKVLELLPGISGGVARVMIGQPFDTIKVRLQVLGAGTALAAKLPPSEVYKDSMDCVRKMIRTEGPLSFYKGTVAPLIGNMILLGIHFPTFSSVRKQLEGDDHYSNFSYTNTLIAGAAAGAAGSLVSTPVELVRTKMQMQRRAALAGSVAGSAASSGAEEFYKGSVDCFKQVLSKHGIKGLYRGFTSTVLRDMQGYAWFFLGYEATVNYFLQNAGPGVHSKADLNYLQVMAAGVVAGFGLWGSMFPIDTIKSKMQADSLAKPQYTTTMDCLRKVLKTEGQVGLWRGFSAAMYRAIPVNAGIFLAVEGSRQGIKWYEENVEHIYGGVVGAAPGAAS

SEQ ID NO: 20 - protein, 354 aa, Gonium pectorale
MSSMTVNDTLNEVEHTPKDPPHKRVLELLPGISGGVARVMIGQPFDTIKTRLQVLGAGTALAAKLPPSEVYKDSMDCVRKMVRSEGPLSFYKGTVAPLFGNMILLGIHFPVFSHVRKQLEGDDHYSNFSYTNALISGAAAGAAGSLVSTPVELVRTKMQMQRRAALAGSAGSAAASSGAEVFYKGSVDCFKQVLSKHGVKGLYRGVTSTVLRDMQGYAWFFLGYEATVNYFLQNAGPGVHSKADLNYLQVMAAGVVAGFGLWGSMFPIDTIKSKMQADSLVKPQYSTTYDCVRKVLKTEGNNGLWRGFSAAMYRAIPVNAGIFLAVEATRQGIKLYEENVEHIYGGVVGTTTAA

SEQ ID NO: 21 - protein, 339 aa, Volvox carteri
MNDTLNQVEHTPPVHKRILDILPGISGGVARVMIGQPFDTIKVRLQVLGQGTALAAQLPPSEVYKDSLDCVRKMVRNEGPLSFYKGTVAPLVGNMVLLGIHFPTFSYVRKQLEGDDHYTNFSYTNTLLSGAAAGAAGSLVSTPVELVRTKMQLQSAASSASDEFYKGSVDCFKQVLSKYGIKGLYRGFTATVLRDMQGYAWFFLGYESTVNYFLQKAGPGLHSKADLNYMQVMSAGVVAGFGLWGSMFPIDTVKSKLQADTLATPQYRSTYDCLSKVLKSEGQAGLWRGFSAAMYRAIPVNAGIFLAVEGTRQGIKWYEENVEHLYGGVVGPATPAATS